(19) United States
(12) Patent Application Publication
(10) Pub. No.: US 2022/0298568 A1
(43) Pub. Date: Sep. 22, 2022

(54) SURFACE STRUCTURING WITH COLLOIDAL ASSEMBLY

(71) Applicant: Pacific Biosciences of California, Inc., Menlo Park, CA (US)

(72) Inventors: Alex Nemiroski, San Diego, CA (US); Gary R. Abel, San Diego, CA (US); Hamed Hosseini Bay, San Diego, CA (US); Joshua Garretson, San Diego, CA (US); Richard D. LeCoultre, San Diego, CA (US); Kurt Patterson, San Diego, CA (US); Tejas Gulati, San Diego, CA (US); Fabian Block, San Diego, CA (US)

(21) Appl. No.: 17/575,094

(22) Filed: Jan. 13, 2022

Related U.S. Application Data
(60) Provisional application No. 63/137,064, filed on Jan. 13, 2021.

(51) Int. Cl. C12Q 1/6874; C12Q 1/6853

(52) U.S. Cl. CPC C12Q 1/6874; C12Q 1/6853

(57) ABSTRACT

Disclosed herein include methods of specifying sites (e.g., sites for colony formation) on a surface (e.g., a planar surface) and generating a flow cell having the sites specified on a surface. Also disclosed are methods of performing sequencing (e.g., sequencing-by-synthesis and sequencing-by-binding) using the flow cell generated and processing (e.g., aligning, orienting, sorting, and assessing quality) images of the flow cell captured during sequencing.

### RELATED APPLICATIONS

The present application claims priority to U.S. Provisional Application No. 63/137,064, filed Jan. 13, 2021. The content of the related application is incorporated herein by reference in its entirety.

### BACKGROUND

### Field

This disclosure relates generally to the field of sequencing, and more particularly to surface structuring for sequencing.

### Background

Flow cells with surfaces having discrete sites for colony formation are used for next generation sequencing. Generating such flow cells with surfaces having “predetermined” and ordered sites or top-down lithography can be expensive and time consuming. There is a need to generate flow cells with surfaces having well-separated sites to enable good data quality, especially at high site densities, at lower costs.

### SUMMARY

Disclosed herein include methods of specifying binding sites on a planar structure. A method of specifying binding sites on a planar structure can comprise: providing a planar structure subsumed in a liquid. The method can comprise: delivering a plurality of particles to a surface of the liquid. The method can comprise: removing the liquid between the plurality of particles and the planar structure, such that the plurality of particles is in contact with the planar structure. The plurality of particles on the surface can specify a plurality of binding sites on the planar structure. The plurality of particles in contact with the planar structure can specify a plurality of binding sites on the planar structure.

Also provided are methods of specifying binding sites on planar structures. A method of specifying binding sites on planar structures can include: providing a plurality of planar structure subsumed in a liquid. The method can include: delivering a plurality of particles to a surface of the liquid. The method can include: removing the liquid between the plurality of particles and the plurality of planar structures, such that the plurality of particles is in contact with the plurality of planar structures. The plurality of particles on the surface and/or in contact with the plurality of planar structures can specify a plurality of binding sites on each of the plurality of planar structures. The pluralities of binding sites on any two planar structures of the plurality of planar structures can be different.

Alternatively or additionally, a method of specifying binding sites on planar structures can contain: providing a planar structure of each of the plurality of planar structures subsumed in a liquid. The method can contain: delivering a plurality of particles to a surface of the liquid. Subsequently, the method can contain: removing the liquid between the plurality of particles and the planar structure, such that the plurality of particles is in contact with the planar structure. The plurality of particles on the surface and/or in contact with the planar structure can specify a plurality of binding sites on the planar structure. The pluralities of binding sites on any two planar structures of the plurality of planar structures can be different.

Removing the liquid can be accomplished through various approaches. In some cases, the removing comprises removing the liquid from a chamber containing the liquid, the planar surface, and the plurality of particles. Alternately, the removing can comprise draining the liquid from a chamber containing the liquid, the planar surface, and the plurality of particles. Heating the liquid is another approach for removing the liquid. In some embodiments, the removing comprises elevating the planar structure above the surface of the liquid such that at least some of the plurality of particles settle onto the planar structure. Removing the liquid in some cases comprises allowing the liquid between the plurality of particles and the planar structure to evaporate. Alternate approaches of removing the liquid are also consistent with the disclosure herein.

A number of liquids are consistent with the disclosure herein. Some suitable liquids have one or more of the following traits: a surface tension suitable for the packing of particles, a viscosity sufficient to allow removal of the liquid from between the plurality of particles and the planar surface without substantial disruption of particle configuration, and a volatility suitable for evaporation of the liquid so as to deposit the plurality of particles on the planar surface without substantial disruption of particle configuration. The liquid is often hydrophilic, although other liquids are consistent with some embodiments. In exemplary embodiments, the liquid comprises one or more of water, an alcohol such as methanol, ethanol, propyl alcohol, butyl alcohol, pentyl alcohol, or a higher order alcohol, optionally fluorinated or otherwise chemically modified; a buffer solution, a salt solution, water, an organic solvent, a polar solvent, a non-polar solvent, an oil, a natural oil, a synthetic oil, an organic oil, a mineral oil, a paraffin oil, a hydrocarbon oil, a non-hydrocarbon oil, a silicone oil, a volatile liquid, or a combination thereof. In some embodiments, a density of the liquid is about 0.1 g/cm³ to about 10 g/cm³. A density of the liquid can be higher than a density of one, one or more, or each, of the plurality of particles. A viscosity of the liquid can be about 10⁻¹ millipascal-second (mPa·s) to about 10⁷ mPa·s. The surface tension of the liquid can be about 10 mN·m⁻¹ to about 500 mN·m⁻¹. Some liquids having properties outside of these ranges are also consistent with the disclosure herein.

In some embodiments, the liquid comprises a spreading agent, a contaminant, or a combination thereof. The spreading agent can comprise an alcohol, such as ethanol, isopropyl alcohol, or isobutyl alcohol. The contaminant can comprise a surfactant, a crowding agent, sucrose, urea, a polyacrylic acid, pyridine aldoxime methyl chloride, or a combination thereof. The surfactant can comprise sodium dodecyl sulfate, Tween, or a combination thereof. The crowding agent can comprise a polyethylene glycol (PEG). The PEG can comprise a PEG with an average molecular of about 200 daltons (e.g., PEG 200) to about 8000 (e.g., PEG 8000). The concentration of a spreading agent (or a contaminant) can have a concentration from about 1% to about 20%.

A range of particles are consistent with the disclosure herein. In some embodiments, the plurality of particles comprises two particles having an identical material. Every particle of the plurality of particles can comprise an identical material. In some embodiments, the plurality of particles comprises two particles having different materials. The plurality of particles can comprise two subsets of particles having different materials. In some cases, the material of one, one or more, or each, of the plurality of particles comprises polydimethylsiloxane (PDMS), polyethylene terephthalate (PET), polybutylene terephthalate (PBT), polymethyl methacrylate (PMMA), polyethylene, polymethylene, polypropylene (PP), polystyrene (PS), poly(vinyl acetate), polyurethane, or a combination thereof.

Particle radius is selected so as to modulate eventual binding site separation distance (such as the minimum separation distance between two binding sites) on the planar surface, assembly of particles into regular or irregular regions on the planar surface, or for other rationales. Particles are often selected such that a radius (or diameter) of one, one or more, or each, of the plurality of particles is about 10⁻⁹ m to about 10⁻⁴ m. A volume of one, one or more, or each, of the plurality of particles can be about 10⁻²⁷ m³ to about 10⁻¹² m³. Some particle populations exhibit some or total uniformity of size, such that two of the plurality of particles have an identical or about identical radius, and/or each of the plurality of particles has an identical radius. Alternately or additionally, some particle populations are heterogeneous, for example so as to modulate the probability or proportion of the particle distribution that is irregular or random. The particles can comprise a first subset of particles having a first identical radius (or diameter) and a second subset of particles having a second identical radius (or diameter). The first identical radius and the second identical radius can be different. The first identical radius (or diameter) and the second identical radius (or diameter) can differ by at least 0.1 μm. The first identical radius and the second identical radius can differ by at least 10% of the first identical radius (or the second identical radius). The first identical radius (or diameter) can be bigger than the second identical radius (or diameter). For example, the first identical radius is 0.5 μm, and the second identical radius can be 0.4 μm. The first identical radius (or diameter) can be smaller than the second identical radius (or diameter). A ratio of a number of the first subset of particles and a number of the second subset of particles is about 1:100 to about 100:1.

Particles are often spherical, such that one, one or more, or each, of the plurality of particles has a spherical shape, although other shapes are also consistent with the disclosure herein. The plurality of particles (or a subset of the particles) deposited on a particular planar surface may comprises about 10⁴ particles to about 10⁸ particles, although numbers greater or less than these values may be employed, such as when smaller or larger planar surfaces are employed or smaller or larger particle sizes are used, for example to modulate binding site minimum distance or pitch.

The particles (or a subset of the particles) can be present at different densities on the surface of a particular liquid and/or on a particular planar surface, such as about 200 k/mm² to about 8,000 k/mm², although alternatives are also consistent with the present disclosure. Non-limiting examples of the density of the particles (or a subset of the particles) on the surface of the liquid and/or on the planar surface are at least 600 k/mm², at least 800 k/mm², at least 1,000 k/mm², and at least 2,000 k/mm².

A range of pitch distances between binding sites are consistent with the disclosure herein. Pitch distance may be selected so as to modulate resolution of signals emanating from individual sites, density of sites on a planar surface, or other criteria. In some cases, a minimum pitch of two, or any two, adjacent binding sites of the plurality of binding sites in a region of regular (or irregular or random) site distribution is about 10⁻⁹ m to about 10⁻⁴ m. Pitches between one binding site and two neighbor (or adjacent) binding sites can be different. For example, three consecutive binding sites in a straight (or substantially straight) line can have different pitches between the two pairs of consecutive binding sites. A pitch between the middle binding site and one neighbor binding site and a pitch between the middle binding site and another neighbor binding site can be different. For example, a binding site can have a number of neighbor binding site (e.g., six neighbor binding sites). The pitches between the binding site and two (or three, four, five, or six) neighbor binding sites can be different. Generally, randomly distributed (or randomly arranged) sites exhibit a larger separation and pitch than regularly arrayed regions. A size of one, or one or more, or each, of the plurality of binding sites can be about 10⁻⁹ m to about 10⁻⁴ m, although sizes outside this range are consistent with some embodiments. The size can be a width, a length, a radius, or a diameter. One, one or more, up to substantially all or each, of the plurality of binding sites has a circular shape. Other shapes of the binding sties are consistent of the present disclosure. The plurality of binding sites on a planar surface can comprise about 10⁴ binding sites to about 10⁸ binding sites.

Delivering the plurality of particles to the surface of the liquid comprises delivering the plurality of particles such that the surface of the liquid comprises a first crystal lattice (or a first irregular array), for example by locally saturating (or partially saturating) the surface of the liquid with the plurality of particles. The first crystal lattice (or first irregular array) can include a subset of particles of the plurality of particles. Delivering the plurality of particles to the surface of the liquid can comprise locally saturating (or partially saturating) the surface of the liquid with the plurality of particles, such that the surface comprises a second crystal lattice (or a second irregular array). The second crystal lattice (or irregular array) can contain a subset of particles of the plurality of particles. The second crystal lattice (or second irregular array) can be separated from the first crystal lattice (or first irregular array) by a disjunction. This disjunction can be a randomly arrayed region that often exhibits a pitch of at least some of the plurality of particles on the surface of the liquid (or the plurality of particles on the planar structure or the result resultant binding sites) that is locally greater or on average greater than that of the crystalline or regularly arrayed first or second region. The disjunction can be a region with no particles on the surface of the liquid (or no particles on the planar structure or no resultant binding sites). Furthermore, a first disjunction or randomly arrayed region often exhibits a particle or site distribution that is unique or distinct relative to a second or all other disjunction or randomly arrayed regions on a planar surface or on any other independently generated planar surface. An irregular array within a field of view usually comprise a plurality of disjunctions and/or a plurality of crystal latices separated by a disjunction. Such field of view may be at least 0.0001 mm², 0.0002 mm², 0.0005 mm², 0.001 mm², 0.002 mm², 0.005 mm², 0.01 mm², 0.02 mm², 0.05 mm², 0.1 mm², 0.2 mm², 0.5 mm², or 1 mm².

A range of local densities are consistent with the disclosure herein. In some embodiments, the plurality of particles (or the resultant binding sites or any subset of the particles or resultant binding sites) is present at a local density of about 200 k/mm² to about 8,000 k/mm². In other embodiments, the plurality of particles is present at a local density of at least 600 k/mm² such as at least 800 k/mm², at least 1,000 k/mm², or at least 2,000 k/mm².

Different configurations of the crystal lattice (such as the arrangement or the relative positions or locations of the particles and the resultant binding sites after etching) may be present in different embodiments. The first crystal lattice often self-assembles such that the first crystal lattice comprises a subset of particles in a hexagonal configuration. The second crystal lattice often self-assembles such that the second crystal lattice comprises a subset of particles in a hexagonal configuration. Seven particles of the subset of particles in the hexagonal configuration can be at six vertices and a center of a hexagon. Each of the six particles at the six vertices of the hexagon can be in contact with the particle at the center of the hexagon and two other particles at the vertices of the hexagon. In some embodiments, the first crystal lattice comprises the subset of particles in an equilateral triangle configuration such that three adjacent non-colinear particles of the first subset of particles form an equilateral triangle. Alternatively or additionally, a first straight line drawn between adjacent particles in the first crystal lattice and any second straight line drawn between adjacent particles in the second crystal lattice are not parallel. Geometries of particles in crystalline regions or resultant binding sites can be chosen so as to increase or even to maximize local two dimensional particle packing density on the liquid surface or later on the planar surface unto which the particles are deposited, or the density of the resultant binding sites.

When, as is often the case, particles assume a configuration having a first crystalline or regular region and a second crystalline region separated by a random or disordered region or ‘crack’ in the crystal lattice, one may observe the following. The first crystal lattice comprises a subset of particles arranged in a plurality of first rows of particles. Particles in each first row of the plurality of rows is observed to be arranged in a linear configuration such that a particle in the first row is in contact with two particles adjacent to the particle in the first row. Two adjacent first rows can be offset by a first offset, which can be more than a radius and less than a diameter of a particle of the plurality of particles (e.g., about the square root of three multiplied by the radius of the particle) in a first direction. The two adjacent rows can be offset by a second offset, such as a diameter of the particle of the plurality of particles in a second direction. The second direction can be perpendicular to the first direction. A particle in one first row can be in contact with two adjacent particles in the other first row. Often, the offset is such that particles in a first row exhibit a pitch that is comparable or substantially identical to the pitch among particles in adjacent first rows, and the resultant binding sites in the first row exhibit a pitch and a separation distance that are comparable or substantially identical to the pitch and the separation distance, respectively, among binding sites in adjacent first rows.

In some embodiments, each particle in an irregular array (e.g., a first irregular array or a second irregular array) is at or greater than a threshold distance away from a nearest neighbor particle of the particle in the irregular array. The threshold distance can be a radius of the particle. An irregular array can comprise no seven neighbor particles that are at six vertices and a center of any hexagon. An irregular array can comprise no six neighbor particles surrounding a 7th particle at six vertices of a hexagon. An irregular array can comprise seven neighbor particles, six of which are at six vertices of a six-sided shape that is not a hexagon and surround a 7th particle of the seven neighbor particles. An irregular array can comprise no particles in a hexagonal configuration, an equilateral triangle configuration, a straight line configuration, or a linear configuration. An irregular array can comprise no particles within at least a threshold distance (e.g., 5 μm) of each other that are in a hexagonal configuration, an equilateral triangle configuration, a straight line configuration, or a linear configuration.

In some embodiments, delivering the plurality of particles comprises delivering a first subset of particles of the plurality of particles to a first location of the surface of the liquid and a second subset of particles of the plurality of particles to a second location of the surface of the liquid simultaneously and/or sequentially. Delivering the plurality of particles can comprise delivering a plurality of subsets (e.g., 5 subsets) of particles of the plurality of particles to different locations at the surface of the liquid simultaneously and/or sequentially.

Consistent with the disclosure herein, the planar structure and the plurality of particles in contact with the planar structure are subjected to etching, for example so as to recapitulate or mirror the particle distribution or configuration on the etched planar surface as binding sites. Such etching may generate: a plurality of retained regions where a substance (or a substance layer or an active site layer) on the planar surface is in contact with (directly or indirectly, such as shielded) or is in close proximity with the plurality of particles and is differentially retained. Etching the planar structure and the plurality of particles in contact with the planar structure can generate: a plurality of etched regions where the substance is not in contact with (directly or indirectly, such as shielded by) the plurality of particles is differentially removed. The substance layer can be hydrophilic, hydrophobic, positively charged, negatively charged, uncharged, or a combination thereof.

The masking layer can be on (or on top of) the substance. The substance on the planar surface at the plurality of retained regions can be in contact (such as indirectly through shielding) with the plurality of particles via the masking layer at the plurality of retained regions. The masking layer at the plurality of retained regions is in contact with the plurality of particles and is differentially retained. The masking layer at the plurality of etched regions is not in contact with the plurality of particles and is differentially removed.

Often, a size of a retained region (and the binding site specified by the retained region) is smaller than a size of a particle that is in contact with the retained region before the etching. A size of a retained region (and the binding site specified by the retained region) is smaller than a size of the particle that is in contact with the retained region after the etching. Alternately, a size of a retained region (and the binding site specified by the retained region) is larger than a size of the particle of the plurality of particles that is in contact with the retained region after the etching. For example, some or all particles may be completely degraded.

Etching often comprises degrading a portion of at least one particle or each particle (or two or more particles, up to substantially all particles) of the plurality of particles. The degrading can determine or impact a size for a binding site of the plurality of binding sites on the planar structure corresponding to a retained region of the plurality of retained regions the at least one particle is in contact with the planar surface. The degrading can determine or impact a separation distance between two adjacent binding sites of the plurality of binding sites on the planar structure. The separation distance can be the shortest distance between the two adjacent binding sites measured between the closest edges of the two adjacent binding sites. In some embodiments, the method comprises removing any of the plurality of particles, or any portion of each particle that remains, if any, subsequent to the etching. The method can comprise removing the masking layer at the plurality of retained regions in contact with the plurality of particles which is differentially retained. In some embodiments, the plurality of etched regions can comprise no substance layer and no masking remaining after etching. Alternatively or additionally, the method can comprise passivating the plurality of etched regions to generate passivated regions. Passivating the plurality of etched regions can occur before removing the masking layer. The passivated regions can be hydrophilic, hydrophobic, positively charged, negatively charged, uncharged, or a combination thereof.

A number of etching approaches are consistent with the disclosure herein. Exemplary approaches include plasma etching, reactive ion etching (RIE), capacitive RIE, inductive RIE, deep reactive ion etching, chemical vapor deposition (CVD), plasma-enhanced CVD (PECVD), or a combination thereof. In some embodiments, the etching comprising isotropic etching, directional etching, vertical etching, or a combination thereof. Etching is variously performed using one gas, or two or more gasses, such as gasses selected from a group consisting of O₂, CF₄, C₂F₆, C₄F₈, CHF₃, SF₆, NF₃, BCl₃, Cl₂, HBr, and Ar. For example, the etching comprises etching using oxygen gas, carbon tetrafluoride gas, or a combination thereof. In some embodiments, the etching comprises performing two or more etching steps. The ratio any two of the two or more gases can be about 1:100 to about 100:1, although alternatives are also consistent with some etching approaches. A mass flow rate of the one gas, a mass flow rate of each of the two or more gases, or a total mass flow rate of the two or more gases, can be about 1 standard cubic centimeter per minute (sccm) to about 100 sccm, or higher or lower. The etching can comprises performing two or more etching steps using different gases. The etching step can comprise performing two or more etching steps using a gas in one etching step and the gas and a second gas in another etching step. The etching can comprise etching at a power of about 10 watt (W) to about 100 W. The etching can comprise etching at a pressure of about 1 millitorr (mT) to about 5000 mT. The etching can comprise etching for about 1 minute (min) to about 10 mins. The etching can comprise etching at a temperature of about 1° C. to about 20° C.

Etching results in the distribution of particles on the planar surface being recapitulated or mirror at least in part on the planar surface as binding sites. In some embodiments, the plurality of retained regions at least partially specifies the plurality of binding sites on the planar structure. In some embodiments, the plurality of etched regions at least partially specifies the plurality of binding sites on the planar structure. Different configurations of the binding sites (for example, the arrangement or the relative positions or locations of the binding sites) are consistent with the present disclosure. In some embodiments, the plurality of retained regions can comprise a first set of seven binding sites of the plurality of binding sites configured into six vertices and a center of a first hexagon. The plurality of retained regions can comprise a second set of seven binding sites of the plurality of binding sites configured into six vertices and a center of a second hexagon. The first hexagon and the second hexagon can share no side. Alternatively or additionally, the plurality of retained regions comprises a first set of three binding sites of the plurality of binding sites configured into three vertices of a first equilateral triangle. The plurality of retained regions can comprise a second set of three binding sites of the plurality of binding sites configured into three vertices of a second equilateral triangle. The first equilateral triangle and the second equilateral triangle can share no side. The first equilateral triangle and the second equilateral triangle can be congruent (e.g., having the same size and shape) equilateral triangles related by a translation and/or a rotation. Alternatively or additionally, the plurality of retained regions comprises binding sites of the plurality of binding sites arranged in a plurality of first rows of binding sites. Binding sites in each first row of the plurality of first rows can be arranged in a linear configuration such that a binding site in the first row is in contact with two binding sites adjacent to the binding site in the first row. Two adjacent first rows of the plurality of first rows can be offset by a first offset which can be more than a radius and less than a diameter of a particle of the plurality of particles, such as about the square root of three multiplied by the radius of the particle, in a first direction of the first plurality of first rows. The two adjacent first rows can be offset by a second offset, for example a diameter of the particle of the plurality of particles, in a second direction of the plurality of first rows. The second direction can be perpendicular to the first direction. The plurality of retained regions can comprise binding sites of the plurality of binding sites arranged in a plurality of second rows of binding sites. The binding sites in each second row of the plurality of second rows can be arranged in a second linear configuration such that a binding site in the second row is in contact with two binding sites adjacent to said binding site in the second row. Two adjacent second rows of the plurality of second rows can be offset by more than a radius and less a diameter of a particle of the plurality of particles, such as about the square root of three multiplied by the radius of the particle, in a first direction of the plurality of second rows. The two adjacent second rows can be offset by the diameter of the particle of the plurality of particles in a second direction of the plurality of second rows perpendicular to the first direction. In some instances, no first row and second row are parallel. In some instances, first row and second row are in contact.

The plurality of binding sites can comprise a subset of binding sites of the plurality of binding sites in a crystal lattice or an irregular array. The plurality of binding sites can comprise two subsets of binding sites of the plurality of binding sites in crystal lattices (e.g., a first crystal lattice and a second crystal lattice) or irregular arrays (e.g., a first irregular array and a second irregular array). The two crystal lattices (or irregular arrays) can be separated by a disjunction. A crystal lattice and an irregular array can be separated by a disjunction. In some embodiments, a crystal lattice is in a hexagonal configuration, an equilateral triangle configuration, a straight line configuration, a linear configuration, or a combination thereof. In some embodiments, an irregular array comprises no binding sites in a hexagonal configuration, an equilateral triangle configuration, a straight line configuration, a linear configuration, or a combination thereof. An irregular array can comprise no particles within at least a threshold distance (e.g., 5 μm) of each other that are in a hexagonal configuration, an equilateral triangle configuration, a straight line configuration, a linear configuration, or a combination thereof.

The method can additionally include delivering a plurality of nucleic acids to the plurality of binding sites. Each of the plurality of nucleic acids can be delivered to a different binding site of the plurality of binding sites. The plurality of nucleic acids can comprise at least one concatemeric nucleic acid. The plurality of nucleic acids can comprise a plurality of concatemeric nucleic acids, such as DNA tiles, and amplification products from rolling circle amplification (RCA).

In some embodiments, the method can comprise performing bridge amplification or rolling circle amplification at the plurality of binding sites. The plurality of nucleic acids being distributed on a plurality of beads is consistent with the present disclosure in some embodiments. Each of the plurality of nucleic acids can be distributed to a different bead of the plurality of beads.

Alternatively or additionally, the method further comprises performing rolling circle amplification (RCA). The method can comprise performing rolling circle amplification in solution or at the plurality of sites. For example, the method can comprise performing rolling circle amplification at the plurality of binding sites using an amplification primer (or a splint primer) attached to one, one or more, or each of the plurality binding sites. The amplification primer (or the splint primer) can be attached to the binding site, for example covalently attached such as by a click chemistry reaction. The amplification primer (or the splint primer) can comprise a first functional moiety capable of participating in a click chemistry reaction. The first functional moiety can comprise, for example, methyltetrazine (MTz). The substance layer can comprise a second functional moiety capable of participating in the click chemistry reaction (e.g., a strain-promoted click chemistry reaction such as a TCO-tetrazing or a DBCO-azide reaction). The second functional moiety can comprise trans-cyclooctene (TCO). The amplification primer or the splint primer can be attached to the binding site via the click chemistry reaction involving the first functional moiety and the second functional moiety.

In some embodiments, the method can comprise delivering a plurality of DNA tiles to the plurality of binding site. Each of the plurality of binding sites can comprise at most one DNA tile of the plurality of DNA tiles. Two binding sites of the plurality of binding sites can comprise two different DNA tiles of the plurality of DNA tiles. Two or more DNA tiles of the plurality of DNA tiles each can comprise an amplification primer (or a splint primer). The method can further comprise performing rolling circle amplification (RCA) at the plurality of binding sites by extending the amplification primer (or the splint primer) attached to each of the two or more DNA tiles using a plurality of template nucleic acids as templates. Alternatively or additionally, the method can comprise performing rolling circle amplification (RCA) in solution. The rolling circle amplification can comprise extending an amplification primer (or a splint primer) using a plurality of template nucleic acid as templates to generate the plurality of nucleic acids prior to delivering the plurality of nucleic acids to the plurality of binding sites. The amplification primer (or the splint primer) can be part of a DNA tile.

In some embodiments, the method can comprise delivering an excitation energy to at least some of the binding sites. The method can comprise collecting an emission energy from at least some of the binding sites.

The present disclosure provides methods of specifying binding sites on a planar structure. A method of specifying binding sites on a planar structure can include one or more of the following: providing a planar structure having deposited thereon an active site layer (or a substance layer) and a masking layer. The method can include: depositing a plurality of beads (or particles) onto the masking layer of the planar structure. The method can include: exposing the planar structure to an etching agent so as to differentially remove the masking layer from regions not shielded by the plurality of beads. The method can include: removing the masking layer and the active site layer from regions not shielded from the etching layer by the plurality of beads. The method can comprise: removing remaining masking layer from regions shielded by the plurality of beads. The method thereby specifies a plurality of binding sites comprising the active site layer remaining

Also disclosed herein are methods of specifying binding sites on a planar structure. A method of specifying binding sites on a planar surface can comprise: providing a planar structure having deposited thereon an active site layer (or a substance layer) and a masking layer. The methods can comprise: depositing a plurality of beads (or particles) onto the masking layer of the planar structure. The methods can comprise: exposing the planar structure to an etching agent. The exposing thereby remove the masking layer and the active site layer from regions not shielded from the etching layer by the plurality of beads. The exposing thereby removes remaining masking layer from regions shielded by the plurality of beads.

The planar structure, the active site layer, and/or the masking layer is often uniform (or substantially uniform) prior to any step of the method, such as the depositing the plurality of beads. The masking layer can be uniform prior to the depositing the plurality of beads. The active site layer can be uniform prior to the depositing the plurality of beads. The planar structure can be uniform prior to the depositing the plurality of beads. That is, through the disclosure herein beads can be deposited onto a planar surface lacking patterning that would otherwise dictate a pre-determined array of binding site positions. Binding sites can be arranged into locally regular distributions such as those described above and elsewhere herein, but the locally regular distributions do not arise from predetermined patterns on the surface. Alternately, the disclosure herein may be used on surfaces that exhibit a predetermined or previously deposited pattern.

The plurality of beads can be in a liquid prior to being deposited onto the masking layer of the planar structure. Depositing the plurality of beads onto the masking layer of the planar structure can comprise removing the liquid between the plurality of beads and the masking layer of the planar structure. The liquid can comprise one or more components, such as a spreading agent and a contaminant, at a concentration such as 1% to about 20%. The spreading agent can comprise an alcohol. The alcohol can comprise ethanol, isopropyl alcohol, isobutyl alcohol or a combination thereof. The contaminant can comprise a surfactant, a crowding agent, sucrose, urea, a polyacrylic acid, pyridine aldoxime methyl chloride, or a combination thereof. The surfactant can comprise sodium dodecyl sulfate, Tween, or a combination thereof. The crowing agent can comprise a polyethylene glycol (PEG), including small molecular weight PEGs, such as PEG 200 and large molecular weight PEGs, such as PEG 8000.

A number of etching agents are consistent with the disclosure herein. Etching agent comprises one gas, or two or more gasses, such as those selected from a group consisting of O₂, CF₄, C₂F₆, C₄F₈, CHF₃, SF₆, NF₃, NFs, BCl₃, Cl₂, CCl₂F₂, HBr, and Ar, although other gasses are contemplated and may also be consistent with the disclosure herein. A ratio of any two of the two or more gases is about 1:100 to about 100:1, although alternatives are also consistent with some etching approaches. A mass flow rate of the one gas, a mass flow rate of each of the two or more gases, or a total mass flow rate of the two or more gases, can be about 1 standard cubic centimeter per minute (sccm) to about 100 sccm, or lower or higher mass flow rate. The etching agent can comprise oxygen gas. Alternatively or additionally, the etching agent comprises carbon tetrafluoride gas. In some embodiments, the etching comprises performing two or more etching steps. The exposing can comprise exposing the planar structure to the etching agent at a power of about 10 watt (W) to about 100 W, or lower or higher power. The exposing can comprise exposing the planar structure to the etching agent at a pressure of about 1 millitorr (mT) to about 5000 mT, or lower or higher pressure. The exposing can comprise exposing the planar structure to the etching agent for about 1 minute (min) to about 10 mins, or shorter or longer time. The exposing can comprise exposing the planar structure to the etching agent at a temperature of about 1° C. to about 20° C., or lower or higher temperature.

The planar structure comprises a surface durable enough to survive the etching process. Exemplary materials for the planar surface include silicon, silicon nitride glass, borosilicate glass, quartz, fused quartz, silica, fused silica, a metal, a ceramic, plastic, or a combination thereof. The masking layer comprises a material that may be etched through use of etching methods and reagents as disclosed herein or known in the art. Exemplary materials of the masking layer comprise aluminum, indium tin oxide, chromium, copper, gallium arsenide, gold, molybdenum, platinum, silicon, silicon dioxide, silicon nitride, silver, tantalum, titanium, titanium nitride, tungsten, or a combination thereof, or other materials consistent with the methods and reagents of the present disclosure or known in the art.

The active site layer comprises a material sufficiently durable to survive the etching process (when in contact with or shielded by the plurality of particles) and able to provide the binding properties necessary to sustain subsequent assay reactions on the surface. Examples include an acrylate functional silane, an aldehyde functional silane, an amino functional silane, an anhydride functional silane, an azide functional silane, a carboxylate functional silane, a phosphonate functional silane, a sulfonate functional silane, an epoxy functional silane, a thiol functional silane, an ester functional silane, a vinyl functional silane, an olefin functional silane, a halogen functional silane, a dipodal silane, or a combination thereof. In some embodiments, the active site layer comprises an aminosilane, a glycidoxysilane, a mercaptosilanes, a functional moiety capable of participating in the click chemistry reaction, trans-cyclooctene (TCO), methyltetrazine (MTz), or a combination thereof. In some instances, the active site layer comprises an aminosilane. Other materials of the active site layer consistent with the methods and compositions of the present disclosure are also contemplated.

Depositing a plurality of beads often comprises packing beads of the plurality of beads into a configuration comprising a first local crystal lattice (or a first irregular array) and a second local crystal lattice (or a second irregular array) separated by a local disjunction. Packing the plurality of beads can comprise arraying the plurality of beads at a density of, for example, about 200 k/mm² to about 8,000 k/mm². Examples of the density of the packed beads include at least 600 k/mm², at least 800 k/mm², at least 1,000 k/mm² or at least 2,000 k/mm². Various numbers of beads deposited are consistent with the present disclosure. The plurality of beads can comprise at least 100,000 beads, at least 1,000,000 beads, at least 10,000,000 beads, or fewer or more beads. Depositing the plurality of beads can comprise depositing a plurality of subsets (e.g., 5 subsets) of beads of the plurality of beads to different locations at the masking layer of the planar structure simultaneously and/or sequentially.

The first crystal lattice and the second crystal lattice of the plurality of beads (and the resultant binding sites) can have (or can be described using or as having) the same or different configurations, such hexagonal configurations, equilateral triangle configurations, straight line configurations, or linear configurations. For example, the first local crystal lattice often comprises a subset of first beads of the plurality of beads in a first hexagonal configuration. Seven first beads of the subset of first beads in the first hexagonal configuration can be at six vertices and a center of a first hexagon. Each of the six first beads at the six vertices of the first hexagon can be in contact with the first bead at the center of the first hexagon and two other first beads at the vertices of the first hexagon. The second local crystal lattice can comprise a second subset of second beads of the plurality of beads in a second hexagonal configuration. Seven second beads of the subset of second beads in the second hexagonal configuration can be at six vertices and a center of a second hexagon. Each of the six second beads at the six vertices of the second hexagon can be in contact with the second bead at the center of the second hexagon and two other second beads at the vertices of the second hexagon. The first hexagonal configuration and the second hexagonal configuration can have different orientations. The first hexagonal configuration and the second hexagonal configuration can have an identical orientation. The first hexagon and the second hexagon can have different orientations. The first hexagon and the second hexagon can have an identical orientation.

As another example, the first local crystal lattice comprises a subset of first beads of the plurality of beads in a first equilateral triangle configuration such that three adjacent non-colinear first beads form a first equilateral triangle. The second crystal lattice can comprise a subset of second beads of the plurality of beads in a second equilateral triangle configuration such that three adjacent non-colinear second beads form a second equilateral triangle. The first equilateral triangle and the second equilateral triangle can share no side.

For example, a first straight line drawn between adjacent beads in the first local crystal lattice and a (or any) second straight line drawn between adjacent beads in the second local crystal lattice are not parallel. Alternatively, a first straight line drawn between adjacent beads in the first local crystal lattice and a (or any) second straight line drawn between adjacent beads in the second local crystal lattice are parallel.

As another example, the first crystal lattice comprises a subset of first beads of the plurality of beads arranged in a plurality of first rows of first beads. First beads in each first row of the plurality of first rows can be arranged in a first linear configuration such that a first bead in the first row is in contact with two first beads adjacent to said first bead in the first row. Two adjacent first rows can be offset by a first offset in a first direction of the first linear configuration. The two adjacent first rows can be offset by a second offset in a second direction of the first linear configuration. The second direction of the first linear configuration can be perpendicular to the first direction of the first linear configuration. The second crystal lattice can comprise a subset of second beads of the plurality of beads arranged in a plurality of second rows of second beads. Second beads in each first row of the plurality of first rows can be arranged in a second linear configuration such that a second bead in the second row is in contact with two second beads adjacent to said second bead in the second row. Two adjacent second rows can be offset by the first offset in a first direction of the second linear configuration. The two adjacent second rows can be offset by the second offset in a second direction of the second linear configuration. The second direction of the second linear configuration can be perpendicular to the first direction of the second linear configuration. The first direction of the first linear configuration and the first direction of the second linear configuration can be different (or the same). The second direction of the first linear configuration and the second direction of the second linear configuration can be different (or the same). In some embodiments, the first offset is more than a radius and less than a diameter of a bead of the plurality of beads (such as about the square root of three multiplied by the radius of the bead). The second offset can be the diameter of the bead of the plurality of beads.

Each bead in an irregular array (e.g., the first irregular array and the second irregular array) can be at or greater than a threshold distance away from a nearest neighbor or neighboring bead of the bead in the irregular array. The threshold distance can be a radius of the bead. The irregular array can comprise no beads in a hexagonal configuration, an equilateral triangle configuration, a straight line configuration, a linear configuration, or a combination thereof. The irregular array can comprise no beads within a threshold distance of each other (e.g., 5 μm) in a hexagonal configuration, an equilateral triangle configuration, a straight line configuration, a linear configuration, or a combination thereof.

The plurality of beads can be randomly deposited, but nonetheless exhibits a set minimum or maximum pitch or bead distance, for example measured from the center of a bead to the center of a neighbor or neighboring bead). Despite being randomly deposited or deposited onto a surface without any predetermined pattern, the plurality of beads often forms a first region having regularly positioned beads and a second region having randomly (or irregularly) positioned beads.

Removing the masking layer and the active site layer comprises, for example, removing bead material and the plurality of beads, or any portion of each bead remaining. A radius (or diameter) of one, one or more, or each, of the plurality of beads can be about 10⁻⁹ m to about 10⁻⁴ m, or shorter or longer. A volume of one, one or more, or each, of the plurality of beads can be about 10⁻²⁷ m³ to about 10⁻¹² m³, or lower or higher. A shortest distance (e.g., measured from the closest edges) between binding sites resultant from two, or any two, adjacent beads of the plurality of beads can be about 10⁻⁹ m to about 10⁻⁴ m, or shorter or longer. A distance between centers of two, or any two, adjacent beads of the plurality of beads can be about 10⁻⁹ m to about 10⁻⁴ m. A distance between centers of two, or any two, adjacent beads of the plurality of beads can be at least twice as large as the shortest distance (e.g., measured from the closest edges) between binding sties resultant from the two adjacent beads.

Disclosed herein include embodiments of a flow cell surface. In some embodiments, the flow cell surface comprises a plurality of binding sites of at least 10,000 binding sites. Each of the plurality of binding sites can be circular. Each of the plurality of binding sites can have a center point and a diameter. In some instances, the flow cell surface includes a plurality of binding sites of at least 10,000 ordered binding sites separated by disjunctions that are not predetermined and/or are randomly (or irregularly) distributed (or arranged). Alternatively or additionally, the flow cell surface contains a plurality of binding sites of at least 10,000 ordered binding sites separated by disjunctions. The configurations of the ordered binding sites and the disjunctions are not predetermined and/or are randomly distributed (or arranged). In some embodiments, the flow cell surface comprises a plurality of binding sites of at least 10,000 binding sites. The plurality of binding sites can include a first plurality of ordered binding sites and a second plurality of ordered binding sites separated by a disjunction that is not predetermined and/or is randomly distributed (or arranged). A first configuration of the first plurality of ordered binding sties and a second configuration of the second plurality of ordered binding sites can be different or the same.

A flow cell surface provided herein can comprise a plurality of binding sites of at least 10,000 binding sites separated by disjunctions. The binding sites and/or the disjunctions can be at positions that are not predetermined. The binding sites and/or the disjunctions can be ordered, can be irregularly distributed, and/or can be randomly distributed. In some instances, configurations of the binding sites can be not predetermined. The disjunctions can be at positions that are not predetermined. The disjunctions (including binding sites therein if any) can be irregularly distributed and/or can be randomly distributed. Binding sites of the plurality of binding sites can be at positions that are not predetermined. The binding sites can be ordered, irregularly distributed (or arranged), and/or randomly distributed (or arranged). In some instances, the plurality of binding site can comprise a first plurality of binding sites and a second plurality of binding sites separated by a disjunction. The position, size, and/or shape of the disjunction can be not predetermined. The disjunction can be randomly distributed. Binding sites of the first plurality of binding sites and/or the second plurality of binding sites can be at positions that are not predetermined, can be ordered, can be irregularly distributed, and/or can be randomly distributed. A subset of binding sites can have a configuration that is not predetermined. A configuration can comprise the number binding sites in the subset of binding sites and/or positions of the binding sites in the subset of binding sites. The first subset of binding sties and the second subset of binding sites can have configurations that are different (or identical).

Different embodiments of the present disclosure contemplate different separations or pitches between any two neighbor binding sites. Separation between any binding site and any nearest neighbor binding site, measured from the center of the first binding site to the center of the nearest neighbor binding site, can be at least twice as large as the diameter of the first binding site. Alternatively or additionally, separation between any binding site and any nearest neighbor binding site, measured from an edge of the first binding site to a center of the nearest neighbor binding site is at least twice as large as the diameter of the first binding site. The two edges are closer than (or at least as close as) separation between any other edge of the first binding site and any edge of the second binding site. The separation can be at least two times, three times, four times, or more as large as the diameter of the first binding site diameter. The separation can be about 10⁻⁹ m to about 10⁻⁴ m, or shorter or longer.

Some, substantially all, or all binding sites being randomly arrayed on the flow cell surface is consistent with the disclosure. For example, at least a portion of the plurality of binding sites is randomly arrayed on the flow cell surface. As another example, the plurality of binding sites is not arrayed on the flow cell surface at a predetermined set of locations. For example, at least a portion of the plurality of binding sites does not share a common pattern. As another example, the plurality of binding sites comprises unpatterned binding sites.

The plurality of binding sites can include a first subset (or plurality) of binding sites and a second subset (or plurality) of binding sites. The first subset of binding sites and the second subset of binding sites can be separated by a disjunction that is not predetermined. A first configuration comprising the first plurality of binding sties (e.g., the relative positions of the binding sites) and a second configuration comprising the second plurality of ordered binding sites can be different. The first subset of binding sites can be in a first crystal lattice or a first irregular array. The second subset of binding sites can be in a second crystal lattice or a second irregular array. The first irregular array and/or the second irregular array can comprise no particles in a hexagonal configuration, an equilateral triangle configuration, a straight line configuration, a linear configuration, or a combination thereof. The first irregular array and/or the second irregular array can comprise no particles within at least a threshold distance of each other that are in a hexagonal configuration, an equilateral triangle configuration, a straight line configuration, a linear configuration, or a combination thereof. The threshold distance can be, for example, 5 μm.

Different local densities of the plurality of binding sites (or a subset of the binding sites) are consistent with the methods and flow cell surfaces of the disclosure. The plurality of binding sites (or a subset of binding sites) can be present at a local density of about 200 k/mm² to about 8,000 k/mm². Different numbers of binding sites are contemplated by the disclosure, such as at least 100,000 binding sites, at least 1,000,000 binding sites. at least 10,000,000 binding sites, or more.

The material of the binding sites can be selected based on the desired properties or applications of the flow cell surface. The binding sites can be hydrophilic, hydrophobic, positively charged, negatively charged, uncharged, or a combination thereof. The material of the flow cell can be selected based on the desired properties or applications of the flow cell surface. A material of the flow cell surface comprises, for example, silicon, silicon nitride glass, borosilicate glass, quartz, fused quartz, silica, fused silica, a metal, a ceramic, plastic, or a combination thereof. The plurality of binding sites can comprise a plurality of nucleic acids. The plurality of nucleic acids can comprise at least one concatemeric nucleic acid. One, at least one, or each, of the plurality of binding sites can comprise one, or at most one, of the plurality of nucleic acids. At least 50% of the plurality of binding sites can comprise at least one nucleic acid of the plurality of nucleic acids and/or one bead of the plurality of beads.

The plurality of nucleic acids can be attached to a plurality of beads. One, at least one, or each, of the plurality of binding sites can comprise one, or at most one, of the plurality of beads. Alternatively or additionally, one, one or more, or each of the plurality of nucleic acids comprises an amplification primer (or a splint primer), an amplification product from rolling circle amplification (RCA), a DNA tile, or a combination thereof. One, one or more, or each of the plurality of nucleic acids can comprise a first functional moiety capable of participating in a click chemistry reaction, such as methyltetrazine (MTz). One, one or more, or each of the plurality of binding sites can comprise a second functional moiety capable of participating in the click chemistry reaction, such as trans-cyclooctene (TCO). The DNA tile can comprise an amplification primer (or a splint primer). The amplification primer (or the splint primer) can be attached to the binding site. The amplification primer (or the splint primer) can be attached to the binding site via the click chemistry reaction involving the first functional moiety and the second functional moiety

Also disclosed herein include embodiments of a flow cell surface. In some embodiments, the flow cell surface comprises a first plurality of reaction sites (e.g., binding sites) and a second plurality of reaction sites adjacent the first plurality of reaction sites. The first plurality of reaction sites can comprise first sets of three reaction sites each configured into three vertices of an identical first equilateral triangle. The second plurality of reaction sites can comprise second sets of three reaction sites each configured into three vertices of a second equilateral triangle. The first equilateral triangle and the second equilateral triangle do not share a parallel side. The first equilateral triangle and the second equilateral triangle can be congruent (e.g., having the same size and shape).

Various reaction sites are consistent with the present disclosure. In some embodiments, the reaction sites comprise an acrylate functional silane, an aldehyde functional silane, an amino functional silane, an anhydride functional silane, an azide functional silane, a carboxylate functional silane, a phosphonate functional silane, a sulfonate functional silane, an epoxy functional silane, a thiol functional silane, an ester functional silane, a vinyl functional silane, an olefin functional silane, a halogen functional silane, a dipodal silane, or a combination thereof. For example, the reaction sites comprise an aminosilane, a glycidoxysilane, a mercaptosilanes, or a combination thereof. As another example, the reaction sites comprise an aminosilane. In some embodiments, the reaction sites comprise emulsion polymerase chain reaction (emPCR) beads. Alternatively or additionally, the reaction sites comprise nucleic acid concatamers and/or bridge-amplified nucleic acid colonies.

A range of reaction site densities are contemplated, for example a local density of about 200 k/mm² to about 2,000 k/mm². Non-limiting exemplary local densities of reaction sites include at least 600 k/mm², at least 800 k/mm², at least 1,000 k/mm², at least 2,000 k/mm², or more. Various numbers of reaction sites are consistent with the disclosure herein, such as at least 100,000 reaction sites, at least 1,000,000 reaction sites, and at least 10,000,000 reaction sites.

The material of the flow cell surface can vary. A material of the flow cell surface can comprise silicon, silicon nitride glass, borosilicate glass, quartz, fused quartz, silica, fused silica, a metal, a ceramic, plastic, or a combination thereof. Each reaction site of the first set of three reaction sites can be separated from every other reaction site of the first set of three reaction sites by 10⁻⁹ m to about 10⁻⁴ m, or shorter or longer. Each reaction site of the second set of three reaction sites can be separated from every other reaction site of the second set of three reaction sites by 10⁻⁹ m to about 10⁻⁴ m, or shorter or longer. In some embodiments, the first plurality of reaction sites is not arrayed in a predetermined configuration. The second plurality of reaction sites is not arrayed in predetermined positions. Various reaction sites are consistent with the disclosure herein. At least 90% of the reaction sites can comprise no more than one nucleic acid tether. At least 90% of the reaction sites can comprise clonal populations of no more than one originating nucleic acid each. An originating nucleic acid on a first reaction site of the two reaction sites and an originating nucleic acid on a second reaction site of the two reaction sites can be distinct.

Disclosed herein include pluralities of flow cell surfaces. In some embodiments, a plurality of flow cell surfaces each comprises at least 10,000 binding sites. No two flow cell surfaces of the plurality of flow cell surfaces share a congruent binding site configuration comprising all of the binding sites on each of the plurality of flow cell surfaces. Alternatively or additional, no corresponding region comprising at least 5% of the binding sites of any two flow cell surfaces of said plurality of flow cell surfaces share a congruent binding site configuration comprising all of the binding sites on each of the corresponding region of plurality of flow cell surfaces. Alternatively or additionally, each of the plurality of flow cell surfaces comprises a region comprising at least 5% of the binding sites on the flow cell surface that does not share a congruent binding site configuration with any region of any other flow cell surface of the plurality of flow cell surfaces. The binding site configuration of a region can comprise all of the binding sites on the region.

Various binding site configurations that are random and/or not predetermined are consistent with the disclosure. For example, two flow cell surfaces of said plurality of flow cell surfaces share a congruent binding site configuration comprising fewer than all of the binding sites on each of the two flow cell surfaces. Two flow cell surfaces of said plurality of flow cell surfaces can share a congruent binding site configuration comprising at most 5% of the binding sites on each of the two flow cell surfaces. As an example, a flow cell surface, or every flow cell surface, comprises at least one plurality of at least three binding sites that is not congruent with any plurality of at least three binding sites on any other flow cell surface. As another example, a flow cell surface, or every flow cell surface, comprises at least one plurality of at least ten binding sites that is not congruent with any plurality of at least ten binding sites on any other flow cell surface. For example, a flow cell surface, or every flow cell surface, comprises at least 5% of the plurality of binding sites on the flow cell surface that is not congruent with any 5% of the plurality of binding sites on any other flow cell surface. As an example, a flow cell surface, or every flow cell surface, comprises at least 10% of the plurality of binding sites on the flow cell that is not congruent with any 10% of the plurality of binding sites on any other flow cell surface.

Additional binding site configurations that are random and/or not predetermined are consistent with the disclosure. As an example, a first flow cell surface of the plurality of flow cell surfaces comprises a first binding site array having two first regions each with a first regular, irregular, or random binding site array configuration and separated by a first region of irregular or random binding site array configuration. A second flow cell surface of the plurality of flow cell surfaces can comprise a second binding site array having two second regions each with a second regular, irregular, or random binding site array configuration and separated by a second region of irregular or random binding site array configuration. The first binding site array and the second binding site array can be distinct. The first binding site array can comprise all of the binding sites on the first flow cell surface. The second binding site array can comprise all of the binding sites on the second flow cell surface.

For example, the first region with the first irregular or random binding site array configuration and the second region with the second irregular or random binding site array configuration are not congruent. The first region with the first irregular or random binding site array configuration can comprise at least one plurality of at least three binding sites that is not congruent with any plurality of at least three binding sites of said second region with the second irregular or random binding site array configuration. The two first regions each with a first regular, irregular, or random binding site array configuration can comprise binding sites that are congruent. The wo first regions each with a first regular, irregular, or random binding site array configuration can comprise binding sites that are not congruent. One of the two first regions with a first regular, irregular, or random binding site array configuration. One of the second regions each with a second regular, irregular, or random binding site array configuration can comprise binding sites that are congruent (e.g., having binding sites at the same or substantially the same relative positions or locations). One of the two first regions each with a first regular, irregular, or random binding site array configuration and one of the second regions each with a second regular, irregular, or random binding site array configuration comprise binding sites that are not congruent.

The number of binding sites in a region of binding site array can vary. In some embodiments, each first region with a first regular, irregular, or random binding site array configuration can comprise at least 500 binding sites. Each second region with a second regular, irregular, or random binding site array configuration can comprise at least 500 binding sites. The first region with a first irregular or random binding site array configuration can comprise at least 500 binding sites. The second region with a second irregular or random binding site array configuration can comprise at least 500 binding sites. In some instances, each first region with a first regular, irregular, or random binding site array configuration comprises at least 5% of the binding sites on the first flow cell surface and/or of the first binding array. Each second region with a second regular, irregular, or random binding site array configuration can comprise at least 5% of the binding sites on the first flow cell surface and/or of the first binding array. The first region with an irregular or random binding site array configuration can comprise at least 5% of the binding sites on the first flow cell surface and/or of the first binding array. The second region with an irregular or random binding site array configuration can comprise at least 5% of the binding sites on the second flow cell surface and/or of the second binding array.

Different materials of the flow cell surface and the binding sites are within the scope of the disclosure. A material of one, one or more, or each, of the plurality of flow cell surfaces can comprise silicon, silicon nitride glass, borosilicate glass, quartz, fused quartz, silica, fused silica, a metal, a ceramic, plastic, or a combination thereof. In some embodiments, the plurality of binding sites on one, one or more, or each, of the plurality of flow cell surfaces comprises a plurality of nucleic acids. Binding sites can be hydrophilic, hydrophobic, positively charged, negatively charged, uncharged, or a combination thereof A nucleic acid can be a concatemeric nucleic acid. One, at least one, or each, of the plurality of binding sites on each of the plurality of flow cell surfaces can comprise one, or at most one, of the plurality of nucleic acids on the plurality of binding sites on the flow cell surface. The plurality of nucleic acids on the plurality of binding sites on each of the plurality of flow cell surfaces can be attached to a plurality of beads. One, at least one, or each, of the plurality of binding sites can comprise one, or at most one, of the plurality of beads. For a flow cell surface, one, one or more, or each of the plurality of nucleic acids on the plurality of binding sites on the flow cell surface comprises an amplification primer (or a splint primer) an amplification product from rolling circle amplification (RCA), a DNA tile, or a combination thereof. One, one or more, or each of the plurality of nucleic acids on the plurality of binding sites on the flow cell surface can comprise a first functional moiety capable of participating in a click chemistry reaction, such as methyltetrazine (MTz). One, one or more, or each of the plurality of binding sites on the flow cell surface comprises a second functional moiety capable of participating in the click chemistry reaction, such as trans-cyclooctene (TCO). The DNA tile can comprise an amplification primer or a splint primer, optionally the amplification primer or the splint primer is attached to the binding site, and optionally the amplification primer or the splint primer is attached to the binding site via the click chemistry reaction involving the first functional moiety and the second functional moiety.

At least 50% of the plurality of binding sites on one, one or more, or each, of the plurality of flow cell surfaces can comprise at least one nucleic acid of the pluralities of nucleic acids and/or one bead of the pluralities of beads.

Also provided are embodiments of a plurality of flow cell surfaces (or a plurality of flow cells) with binding sites having different configurations. In some embodiments, each flow cell surface (or a surface of a flow cell) comprises at least 10,000 ordered, irregular, or random binding sites separated by disjunctions that are at non-predetermined locations and/or are randomly distributed (or arranged). No two flow cell surfaces comprise an identical configuration of the disjunctions on the flow cell surface. In some instances, each flow cell surface (or a surface of a flow cell) comprises at least 10,000 ordered, irregular, or random binding sites separated by disjunctions. Configurations (e.g., the relative positions or locations) of the ordered, irregular, or random binding sites and the disjunctions are not predetermined and/or are randomly distributed (or arranged). No two flow cell surfaces comprise an identical configuration of the ordered, irregular, or random binding sites and disjunctions. Other embodiments provide each flow cell surface (or a surface of a flow cell) comprising at least 10,000 ordered, irregular, or random binding sites separated by irregular or random regions of binding sites at non-predetermined locations and/or are randomly distributed (or arranged). No two flow cell surfaces comprise an identical configuration of the irregular or random regions on the flow cell surface. Alternatively or additionally, each flow cell surface (or a surface of a flow cell) comprises at least 10,000 ordered, irregular, or random binding sites separated by irregular or random regions of binding sites. Configurations of the ordered, irregular, or random binding sites and the irregular or random regions of bindings comprise binding sites that are not predetermined and/or are ordered, are irregularly distributed, or are randomly distributed (or arranged). No two flow cell surfaces comprise an identical configuration of the ordered, irregular, or random binding sites and the irregular or random regions of binding sites. In some instances, each flow cell surface (or a surface of a flow cell) comprises at least 10,000 binding sites in regular regions of binding sites and irregular or random regions of binding sites separating the regular regions of binding sites. The regular regions and the irregular or random regions are at non-predetermined locations and/or are randomly distributed (or arranged). No two flow cell surfaces comprise an identical configuration of the regular, irregular, or random regions and/or the irregular or random regions.

Disclosed herein include methods of aligning a plurality of flow cell images. In some embodiments, a method of aligning a plurality of flow cell images is under control of a processor and comprises: obtaining a plurality of flow cell images from a flow cell surface having a first regular, irregular, or random binding site region and a second regular, irregular, or random binding site region separated by an irregular or random binding site region. The method can comprise: aligning the irregular or random binding site region in the plurality of flow cell images to align the plurality of flow cell images. In some embodiments, a method of aligning a plurality of flow cell images is under control of a processor and comprises: obtaining a plurality of flow cell images from a flow cell surface having regular, irregular, or random binding site regions separated by irregular or random binding site regions. The method can comprise: aligning the irregular or random binding site regions in the plurality of flow cell images to align the plurality of flow cell images. In some embodiments, a method of aligning a plurality of flow cell images is under control of a processor and comprises: obtaining a plurality of flow cell images from a flow cell surface comprising ordered binding site separated by disjunctions. The method can comprise: aligning the disjunctions in the plurality of flow cell images to align the plurality of flow cell images. The aligning can comprise translating one flow cell image relative to a second flow cell image. The aligning can comprise rotating one image relative to a second image.

Different types of flow cell images and different numbers of flow cell images are consistent with the present disclosure. The plurality of flow cell images can comprise florescence emission signals emitted from binding sites of the first regular, irregular, or random binding site region, the second regular, irregular, or random binding site region, and the irregular or random binding site region. The plurality of flow cell images comprises at least 20 flow cell images, at least 200 flow cell images, or more.

Also provided herein are methods of sorting a plurality of flow cell images. In some embodiments, a method of sorting a plurality of flow cell images is under control of a processor and comprises: obtaining a plurality of flow cell images. The method can comprise: identifying a first regular, irregular, or random binding site region and a second regular, irregular, or random binding site region separated by an irregular or random binding site region in each of the plurality of cell images. The method can comprise: sorting the plurality of flow cell images such that flow cell images having an identical irregular or random binding site region are assigned to a common group and two flow cell images having different irregular or random binding site region are assigned to different common groups.

In some embodiments, a method of sorting a plurality of flow cell images is under control of a processor and comprises: obtaining a plurality of flow cell images. The method can comprise: identifying an irregular or random binding site region in each of the plurality of flow cell images that separates a first regular, irregular, or random binding site region and a second regular, irregular, or random binding site region in the flow cell image. The method can comprise: sorting the plurality of flow cell images such that flow cell images having an identical irregular or random binding site region are assigned to a common group and two flow cell images having different irregular or random binding site region are assigned to different common groups. The method can comprise one or more additional steps disclosed herein. For example, the method can further comprise orienting flow cell images of the plurality of flow cell images in each common group such that first binding sites in the flow cell images in the each common group are aligned and the second binding sites in the flow cell image images in the each common group are aligned. Flow cell images having different irregular or random binding site regions can be assigned to different groups.

The present disclosure provides methods of performing quality assessment on an image. In some embodiments, a method of performing quality assessment is under control of a processor and comprises: receiving an image collected from a surface comprising a plurality of binding sites. The method can comprise: identifying a first signal from a first binding site. The method can comprise: identifying a second signal from a second binding site. The method can comprise: determining a distance separating the first binding site from the second binding site. The method can comprise: negatively assessing the image if the distance is below a threshold.

Negatively assessing the image can be accomplished through various approaches. Non-limiting examples of negatively assessing the image include discarding the image; discarding the first signal and the second signal from the image; discarding any signal from the first binding site and any signal from the second binding site; tagging at least one of the first binding site and the second binding site as out of focus; and refocusing at least one of the first binding site and the second binding site. The refocusing can vary. Non-limiting examples of refocusing include reducing a size of the first signal; reducing a size of the first signal and a size of the second signal; increasing an intensity of the first signal; increasing an intensity of the first signal and an intensity of the second signal; and reducing a size of the first signal and a size the second signal and increasing an intensity of the first signal and an intensity of the second signal.

Another example of negatively assessing the image includes determining an out-of-focus value based on the distance and the threshold. A further example of negatively assessing the image includes determining an out-of-focus value based on the distance and an ideal distance. The out-of-focus value can be a ratio of the distance and the threshold, a ratio of the distance and the ideal distance threshold, or a combination thereof. The out-of-focus value can be a difference of the distance and the threshold, a difference of the distance and the ideal distance threshold, or a combination thereof. Negatively assessing the image can include: causing a refocused image to be collected from the surface based on the out-of-focus value; and/or receiving a refocused image collected from the surface based on the out-of-focus value. Negatively assessing the image can include: adjusting a focus of an imaging system that collected the image based on the out-of-focus value; and/or causing the imaging system to adjust a focus of the imaging system based on the out-of-focus value. Negatively assessing the image can include: causing a refocused image to be collected from the surface. Negatively assessing the image can comprise receiving a refocused image to collected from the surface.

Determining the distance can be accomplished through various approaches. For example, determining the distance comprises measuring a distance from the first binding site to the second binding site using a central point of the first binding site and a central point of the second binding site. As another example, determining the distance comprises measuring a distance from the first binding site to the second binding site using an edge of the first binding site and an edge of the second binding site.

Various binding site configurations that are random and/or not predetermined are consistent with the disclosure. For example, at least a first portion of said plurality of binding sites is regularly, irregularly, or randomly arrayed on said flow cell surface. At least a second portion of said plurality of binding sites can be irregularly or randomly arrayed on said flow cell surface. As an example, at least a portion of said plurality of binding sites is not arrayed on said flow cell surface in a predetermined set of locations. As another example, at least a portion of said plurality of binding sites does not share a common pattern. As a further example, at least said plurality of binding sites comprises unpatterned binding sites. For example, said plurality of binding sites is randomly arrayed. As one example, said plurality of binding sites is arrayed so as to form a first region having regularly positioned (or ordered) binding sites and a second region having randomly (or irregularly) positioned binding sites.

Disclosed herein include methods of orienting a plurality of flow cell images. In some embodiments, a method of orienting a plurality of flow cell images is under control of a processor and comprises: identifying an irregular or random colony region of a plurality of colonies common to a plurality of the flow cell images. The method can comprise: orienting one or more of the plurality of flow cell images such that the irregular or random colony region is aligned among the plurality of flow cell images.

Various distributions of the colonies are consistent with the disclosure herein. For example, the plurality of colonies on the flow cell is distributed such that no two colonies are closer than a minimum distance from one another. For example, the plurality of colonies on the flow cell is distributed such that no two colonies, from a center of each of the two colonies, are closer than a minimum distance from one another. For example, the plurality of colonies on the flow cell is distributed such that no two colonies, from an edge of each of the two colonies, are closer than a minimum distance from one another The minimum distance can be about 10⁻⁹ m to about 10⁻⁴ m, or shorter or longer. A size of one, one or more, or each, of the plurality of colonies can be about 10⁻⁹ m to about 10⁻⁴ m, or shorter or longer. The size can be a radius or a diameter. A distance of one of the plurality of colonies and a nearest neighbor colony of the plurality of colonies can be about 10⁻⁹ m to about 10⁻⁴ m, or longer or shorter. A distance of one of the plurality of colonies and a nearest neighbor colony of the plurality of colonies is at least twice (or more) as large as a size of the colony.

The disclosure contemplates numerous colony densities and numbers of colonies. For example, the plurality of colonies is present at a density of about 200 k/mm² to about 2,000 k/mm². For example, the plurality of colonies is present at a density of at least 600 k/mm², at least 800 k/mm², at least 1,000 k/mm², or more. For example, the plurality of colonies comprises at least 100,000 colonies, at least 1,000,000 colonies, at least 10,000,000 colonies, or more.

The plurality of flow cell images can be collected from one or more flow cells. For example, the plurality of flow cell images is collected from a single flow cell. The single flow cell can comprise at least one region of regularly irregularly, or randomly arrayed colonies and at least one region of irregularly or randomly arrayed colonies. A flow cell image of the plurality of flow cell images lacking the irregular colony region can be discarded from the plurality of flow cell images. As another example, the plurality of flow cell images is collected from a plurality of flow cells. The plurality of flow cells can comprise flow cells each having at least one region of regularly irregularly, or randomly arrayed colonies and at least one region of irregularly or randomly arrayed colonies, and no two flow cells share an identical array of irregularly or randomly arrayed colonies. The method can comprise sorting the plurality of images such that images having a common region of irregularly or randomly arrayed colonies are grouped together. The method can comprise: sorting images such that images lacking a common region of irregularly or randomly arrayed colonies are differentially grouped.

Disclosed herein include flow cell imaging systems. In some embodiments, a flow cell imaging system comprises: a flow cell having distributed thereon a plurality of binding sites, at least some of the binding sites are randomly distributed. The flow cell imaging system can comprise: an excitation source to excite fluorophores at the binding sites. The flow cell imaging system can comprise: an image digitization interface comprising a plurality of pixels. The plurality of binding sites can be distributed such that no two binding sites generate emission signals that are assigned to a common pixel. Alternatively or additionally, at least some of the binding sites are regularly, irregularly, or randomly distributed. The plurality of binding sites can be present at various densities, such as a density of about 200 k/mm² to about 8,000 k/mm², a density of about 200 k/mm² to about 2,000 k/mm², at a density of at least 600 k/mm², at least 800 k/mm², at least 1,000 k/mm², or more The number of binding sites can vary, such as at least 100,000 binding sites, at least 1,000,000 binding sites, at least 10,00,000 binding sites, or more.

Also provided herein include a nucleic acid tile comprising a scaffold nucleic acid and a plurality of staple oligonucleotides. One, one or more, or each of the plurality of staple oligonucleotides can comprise at least two binding domains hybridized to different regions of the scaffold nucleic acid, thereby forming a double-crossover motif comprising the scaffold nucleic acid and the staple oligonucleotide. The plurality of staple oligonucleotides can comprise a first anchor oligonucleotide that protrudes from a first face of the nucleic acid tile. The nucleic acid tile can comprise a deoxyribonucleic acid (DNA). The scaffold nucleic acid can comprise a DNA. The plurality of staple oligonucleotides can comprise DNAs.

In some embodiments, the scaffold nucleic acid forms a pseudocircle by the hybridization of the plurality of staple oligonucleotides. The nucleic acid tile can be pseudocircular in shape. The scaffold nucleic acid can be about 5 kilobases (kb) to about 50 kb in length. The scaffold nucleic acid can comprise genomic DNA, for example, genomic DNA of M13mp18 bacteriophage. The plurality of staple oligonucleotides can comprise a second anchor oligonucleotide that protrude from a second face of the nucleic acid tile. The first anchor oligonucleotide and the second anchor oligonucleotide can protrude from opposite faces of the nucleic acid tile.

An anchor oligonucleotide can comprise a splint sequence. The splint sequence can be at the 3′ end of the anchor oligonucleotide. The anchor oligonucleotide can comprise a spacer sequence. The spacer sequence can be 5′ to the splint sequence. The spacer sequence can comprise a poly-T sequence. The spacer sequence can be about three to about 10 nucleotides in length. The splint sequence can comprise a first splint sequence and a second splint sequence. The melting temperature (Tm) of the first splint sequence can be higher than Tm of the second splint sequence. The plurality of staple oligonucleotides can comprise a plurality of capture oligonucleotides each comprising a first capture sequence. The first capture sequence can comprise the second splint sequence. The capture oligonucleotide may not comprise the first splint sequence. Capture oligonucleotides of the plurality of capture oligonucleotides can be identical. One, one or more, or each of the plurality of capture oligonucleotides can comprise a cleavable site, such as a cleavable nucleotide. One, one or more, or each of the plurality of capture oligonucleotides can comprise a second capture sequence.

Disclosed herein include methods of forming a nucleic acid tile. In some embodiments, a method of forming a nucleic acid tile comprises providing a staple solution comprising the plurality of staple oligonucleotides. The method can comprise providing a scaffold solution comprising a scaffold nucleic acid. The method can comprise combining the staple solution and the scaffold solution to form a reaction solution. The method can comprise subject the reaction solution to thermal annealing, thereby forming the nucleic acid tile. The reaction solution can differ. For example, the reaction solution comprises each of the plurality of staple oligonucleotides at about 100 nanomolar (nM), the scaffold nucleic acid at about 10 millimolar (mM), tris acetate at about 40 mM, magnesium chloride at about 12.5 mM, and/or EDTA at about 0.1 mM. The reaction solution can have a volume of about 50 microliter (uL). The method can comprise purifying the nucleic acid tile from molecules of the scaffold nucleic acid and molecules of the plurality of staple oligonucleotides that are not parts of molecules of the nucleic acid tile. The method can comprise purifying the nucleic acid tile comprises size-based purification.

A method of rolling circle amplification (RCA) is contemplated herein. A method of RCA can comprise providing a nucleic acid tile. The nucleic acid tiles may be bound to binding sites of a structured surface as described herein. The method can comprise providing a target nucleic acid. The method can comprise circularizing the target nucleic acid using the splint sequence of the nucleic acid tile. The method can comprise performing rolling circle amplification by extending the splint sequence using the target nucleic acid as the template to generate the nucleic acid tile each comprising concatemeric copies of the target nucleic acid.

A method of RCA can comprise providing a plurality of nucleic acid tiles. The method can comprise providing a plurality of target nucleic acids. The method can comprise hybridizing each of the plurality of target nucleic acids to the splint sequence of a nucleic acid tile of the plurality of nucleic acid tiles. The method can comprise circularizing the target nucleic acid hybridized to the splint sequence of the nucleic acid tile. The method can comprise performing rolling circle amplification by extending the splint sequence of the nucleic acid tile using the target nucleic acid hybridized thereto as a template to generate a nucleic acid cluster comprising the nucleic acid tile with the splint sequence thereof extended to include concatemeric copies of the target nucleic acid.

The method can further comprise depositing the nucleic acid tiles onto binding sites of a flow cell surface before RCA. The method can further comprise depositing the nucleic acid clusters onto binding sites of a flow cell surface after RCA. At least 50% of the plurality of binding sites can each comprise at most one nucleic acid tile (or at most one nucleic acid cluster).

In some instances, the method further comprises removing the plurality of capture oligonucleotides by cleaving the cleavable sites. In some instances, a target nucleic acid of the plurality of target nucleic acids is hybridized to the first splint sequence of the anchor oligonucleotide and the first capture sequence (or the second splint sequence) of a capture oligonucleotide of the plurality of capture oligonucleotides. The method can comprise hybridizing a release oligonucleotide to the capture oligonucleotide. As a result, the target nucleic acid can hybridize to the first splint sequence and the second splint sequence of the anchor oligonucleotide. In some embodiments, a first target nucleic acid of the plurality of target nucleic acids is hybridized to the first splint sequence of the anchor oligonucleotide and the first capture sequence (or the second splint sequence) of a first capture oligonucleotide of the plurality of capture oligonucleotides. A second target nucleic acid of the plurality of target nucleic acids can be hybridized to the second splint sequence of a second capture oligonucleotide of the plurality of capture oligonucleotides. The method can comprise subjecting the nucleic acid tile to thermal cycling. As a result, the second target nucleic acid is released from the second capture oligonucleotide. And the first target nucleic acid is hybridized to the first splint sequence and the second splint sequence of the anchor oligonucleotide. In some embodiments, a first target nucleic acid is hybridized to the first splint sequence and the second splint sequence of the anchor oligonucleotide. A second target nucleic acid of the plurality of target nucleic acids can be hybridized to the second splint sequence of a second capture oligonucleotide of the plurality of capture oligonucleotides. The method can further comprise subjecting the nucleic acid tile to thermal cycling. As a result, the second target nucleic acid is released from the second capture oligonucleotide.

In some instances, a method of binding a concatemer to a structured surface, may include: providing a plurality of asymmetric DNA tiles; providing a plurality of concatemers; binding individual concatemers to a first side of individual DNA tiles in solution; and depositing the DNA tiles on a surface such that a second side of the individual DNA tiles binds to the surface while the first side of the individual DNA tiles remains bound to a concatemer. The DNA tiles may be deposited onto binding sites of a flow cell surface disclosed herein.

In some instances, the binding sites of the flow cell surface, or of a plurality of flow cell surfaces, are hydrophilic, hydrophobic, positively charged, negatively charged, uncharged, comprise a functional moiety for covalent attachment optionally wherein the functional moiety is capable of participating in a click chemistry reaction, comprise an affinity biomolecule optionally wherein the affinity biomolecule is biotin or streptavidin, comprise an intermediate nucleic acid, or a combination thereof. A method may include depositing concatemers on such binding sites, optionally wherein the concatemers are rolling circle amplification (RCA) products. The method may further include performing RCA in solution by extending an amplification primer or a splint primer with a plurality of templates to generate the concatemers prior to depositing the concatemers. RCA may include incorporating nucleotides functionalized to covalently bind the binding sites of the flow cell, optionally wherein the nucleotides comprise a functional moiety capable of participating in a click chemistry reaction. The amplification primer or splint primer may be functionalized to covalently bind the binding sites, or to an intermediate nucleic acid such as a DNA tile. The deposited concatemer may be bound to the binding sites by covalent binding or hybridization to an intermediate nucleic acid bound to the binding sites, optionally wherein the intermediate nucleic acid is covalently bound to the binding sites, optionally wherein the intermediate nucleic acid is a DNA tile. The method may include forming concatemers on the binding sites, optionally wherein the concatemers are RCA products. Concatemers may be formed by RCA from a splint primer sequence covalently attached to the binding site or presented by a DNA tile bound to the binding site, for example as an alternative to RCA in solution. Any method described herein may further comprising sequencing a nucleic acid, such as sequencing concatemers bound to binding sites of a flow cell described herein.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIGS. 1A-1B show a non-limiting exemplary plot illustrating an inverse relationship of spot (or site) density and average distance between spots. For any given spot density, ordered spots have a shorter average distance between spots as compared to random spots.

FIG. 2A1 shows a non-limiting exemplary method of surface structuring for high-density spots (or sites) ordered in a non-predetermined manner. FIG. 2A2 shows another non-limiting exemplary method of surface structuring for high-density spots (or sites) ordered in a non-predetermined manner. FIGS. 2B and 2C show a non-limiting exemplary scanning electron microscope (SEM) images of structured surfaces (e.g., flow cell surfaces) having ordered spots (or sites) at a high density, where the ordered spots were generated using colloidal self-assembly in a non-predetermined manner.

FIGS. 3A-3D are non-limiting exemplary schematic illustrations of top views of planar structures after the particles come into contact with the planar structures showing crystal lattices of particle configurations separated by disjunctions.

FIGS. 4A-4D show non-limiting exemplary schematic illustrations of top views of planar structures comprising binding sites after the etching process is performed, for example, on the planar structures with particles shown in FIGS. 3A-3D respectively.

FIGS. 5A, 5B1-5B4, 5C1-5C2, and 5D1-5D2 illustrate colloidal self-assembly.

FIGS. 6A, 6B1-6B3, 6C1-6C2, and 6D illustrate structured surfaces generated after colloidal bead assembly and etching of the beads, the mask layer, and the substance/active site layer.

FIGS. 7A-7C and 7D1-7D2. emPCR bead deposition and sequencing.

FIG. 8 shows the binding sites generated using beads with 0.8 μm diameter and beads with 1.0 μm diameter at a ratio of 2:5 respectively (by the number of beads). The binding sites are irregularly arranged such that an ideal hexagonal grid (FIG. 8, far left panel) does not align with the binding sites (FIG. 8, far right panel).

FIG. 9A shows that density of the spots are not significantly sensitive to disorder. Disordered surface shows at most a 10-15% reduction from perfectly ordered arrays. FIG. 9B shows array irregularities resulting from monodispersity with 1.0 μm-diameter beads, polydispersity with 0.8 μm-diameter beads and 1.0 μm beads at 1:10 ratio, and polydispersity with 0.8 μm-diameter beads and 1.0 μm beads at 2:5 ratio.

FIGS. 10A-10B show that various additives can be used for improving deposition of RCA products on structured (regular or irregular) surface. FIG. 10B shows spot counts and spot intensities of deposited clusters. Higher and tight distributions of spot count and intensity are preferred.

FIG. 11 shows a schematic illustration of rolling circle amplification (RCA) for cluster generation and deposition.

FIG. 12 compares rolling circle amplification (RCA) products at binding sites with active chemistry (e.g., with amplification primers attached to the binding sites) and randomly on a flow cell surface with active chemistry. DNA clusters could be concatemers such as RCA products, and could have an DNA tile between a RCA product and the surface.

FIG. 13 shows a schematic illustration of a DNA tile design (left) and atomic force microscope (AFM) image and height profiles of circular (or pseudocircular) DNA tiles (right).

FIG. 14 shows an overview of DNA tiles for structured on-surface clustering.

FIGS. 15A-15B compare clustering on unstructured DNA tile surface (FIG. 15A) and on structured DNA tile surface (FIG. 15B).

FIG. 16 shows improving library capture kinetics using additional capture oligonucleotides.

FIG. 17 shows toehold-mediated strand displacement to drive library migration to splint.

FIG. 18 shows thermal cycling to improve capture yield.

FIG. 19 is a block diagram of an illustrative computing system configured to implement any method of processing one or more flow cell images, including aligning flow cell images, orienting flow cell images, sorting flow cell images, and performing quality assessment on flow cell images.

Throughout the drawings, reference numbers may be re-used to indicate correspondence between referenced elements. The drawings are provided to illustrate example embodiments described herein and are not intended to limit the scope of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments can be utilized, and other changes can be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods of specifying binding sites on a planar structure. A method of specifying binding sites on a planar structure can comprise: providing a planar structure subsumed in a liquid. The method can comprise: delivering a plurality of particles to a surface of the liquid. The method can comprise: removing the liquid between the plurality of particles and the planar structure, such that the plurality of particles is in contact with the planar structure. The plurality of particles on the surface can specify a plurality of binding sites on the planar structure. The plurality of particles in contact with the planar structure can specify a plurality of binding sites on the planar structure.

Also provided are methods of specifying binding sites on planar structures. A method of specifying binding sites on planar structures can include: providing a plurality of planar structure subsumed in a liquid. The method can include: delivering a plurality of particles to a surface of the liquid. The method can include: removing the liquid between the particles and the plurality of planar structures, such that the plurality of particles is in contact with the plurality of planar structures. The plurality of particles on the surface and/or in contact with the plurality of planar structures can specify a plurality of binding sites on each of the plurality of planar structures. The pluralities of binding sites on any two planar structures of the plurality of planar structures can be different.

The present disclosure provides methods of specifying binding sites on a planar structure. A method of specifying binding sites on a planar structure can include one or more of the following: providing a planar structure having deposited thereon an active site layer (or a substance layer) and a masking layer. The method can include: depositing a plurality of particles onto the masking layer of the planar structure. The method can include: exposing the planar structure to an etching agent so as to differentially remove the masking layer from regions not shielded by the plurality of beads. The method can include: removing the masking layer and the active site layer from regions not shielded from the etching layer by the plurality of beads. The method can comprise: removing remaining masking layer from regions shielded by the plurality of beads. The method thereby specifies a plurality of binding sites comprising the active site layer remaining

Also disclosed herein are methods of specifying binding sites on a planar structure. A method of specifying binding sites on a planar surface can comprise: providing a planar structure having deposited thereon an active site layer (or a substance layer) and a masking layer. The methods can comprise: depositing a plurality of particles onto the masking layer of the planar structure. The methods can comprise: exposing the planar structure to an etching agent. The exposing thereby removes the masking layer and the active site layer from regions not shielded from the etching layer by the plurality of beads. The exposing thereby removes remaining masking layer from regions shielded by the plurality of beads.

Disclosed herein include embodiments of a flow cell surface. In some embodiments, the flow cell surface comprises a plurality of binding sites of at least 10,000 binding sites. Each of the plurality of binding sites can be circular. Each of the plurality of binding sites can have a center point and a diameter. In some instances, the flow cell surface includes a plurality of binding sites of at least 10,000 ordered binding sites separated by disjunctions that are not predetermined and/or are randomly distributed (or arranged). Alternatively or additionally, the flow cell surface contains a plurality of binding sites of at least 10,000 ordered binding sites separated by disjunctions. The configurations of the ordered binding sites and the disjunctions are not predetermined and/or are randomly distributed (or arranged). In some embodiments, the flow cell surface comprises a plurality of binding sites of at least 10,000 binding sites. The plurality of binding sites can include a first plurality of ordered binding sites and a second plurality of ordered binding sites separated by a disjunction that is not predetermined and/or is randomly distributed (or arranged). A first configuration of the first plurality of ordered binding sties and a second configuration of the second plurality of ordered binding sites can be different or the same.

Also disclosed herein include embodiments of a flow cell surface. In some embodiments, the flow cell surface comprising a first plurality of reaction sites and a second plurality of reaction sites adjacent the first plurality of reaction sites. The first plurality of reaction sites can comprise first sets of three reaction sites each configured into three vertices of an identical first equilateral triangle. The second plurality of reaction sites can comprise second sets of three reaction sites each configured into three vertices of a second equilateral triangle. The first equilateral triangle and the second equilateral triangle do not share a parallel side. The first equilateral triangle and the second equilateral triangle are congruent (e.g., having the same size and shape).

Disclosed herein include pluralities of flow cell surfaces. In some embodiments, a plurality of flow cell surfaces each comprises at least 10,000 binding sites. No two flow cell surfaces of the plurality of flow cell surfaces share a congruent binding site configuration comprising all of the binding sites on each of the plurality of flow cell surfaces. Alternatively or additional, no corresponding region comprising at least 5% of the binding sites of any two flow cell surfaces of said plurality of flow cell surfaces share a congruent binding site configuration comprising all of the binding sites on each of the corresponding region of plurality of flow cell surfaces. Alternatively or additionally, each of the plurality of flow cell surfaces comprises a region comprising at least 5% of the binding sites on the flow cell surface that does not share a congruent binding site configuration with any region of any other flow cell surface of the plurality of flow cell surfaces. The binding site configuration of a region can comprise all of the binding sites on the region.

Also provided are embodiments of a plurality of flow cell surfaces (or a plurality of flow cells) with binding sites having different configurations. In some embodiments, each flow cell surface (or a surface of a flow cell) comprises at least 10,000 ordered binding sites separated by disjunctions that are at non-predetermined locations and/or are randomly distributed (or arranged). No two flow cell surfaces comprise an identical configuration of the disjunctions on the flow cell surface. In some instances, each flow cell surface (or a surface of a flow cell) comprises at least 10,000 ordered binding sites separated by disjunctions. Configurations (e.g., the relative positions or locations) of the ordered binding sites and the disjunctions are not predetermined and/or are randomly distributed (or arranged). No two flow cell surfaces comprise an identical configuration of the ordered binding sites and disjunctions. Other embodiments provide each flow cell surface (or a surface of a flow cell) comprising at least 10,000 ordered binding sites separated by irregular regions of binding sites at non-predetermined locations and/or are randomly distributed (or arranged). No two flow cell surfaces comprise an identical configuration of the irregular regions on the flow cell surface. Alternatively or additionally, each flow cell surface (or a surface of a flow cell) comprises at least 10,000 ordered binding sites separated by irregular regions of binding sites. Configurations of the ordered binding sites and the irregular regions of bindings are not predetermined and/or are randomly distributed (or arranged). No two flow cell surfaces comprise an identical configuration of the ordered binding sites and the irregular regions of binding sites. In some instances, each flow cell surface (or a surface of a flow cell) comprises at least 10,000 binding sites in regular regions of binding sites and irregular regions of binding sites separating the regular regions of binding sites. The regular regions and the irregular regions are at non-predetermined locations and/or are randomly distributed (or arranged). No two flow cell surfaces comprise an identical configuration of the regular regions and/or the irregular regions.

A flow cell surface provided herein can comprise a plurality of binding sites of at least 10,000 binding sites separated by disjunctions. The binding sites and/or the disjunctions can be at positions that are not predetermined. The binding sites and/or the disjunctions can be ordered, can be irregularly distributed, and/or can be randomly distributed. In some instances, configurations of the binding sites can be not predetermined. The disjunctions can be at positions that are not predetermined. The disjunctions (including binding sites therein if any) can be irregularly distributed and/or can be randomly distributed. Binding sites of the plurality of binding sites can be at positions that are not predetermined. The binding sites can be ordered, irregularly distributed (or arranged), and/or randomly distributed (or arranged). In some instances, the plurality of binding site can comprise a first plurality of binding sites and a second plurality of binding sites separated by a disjunction. The position, size, and/or shape of the disjunction can be not predetermined. The disjunction can be randomly distributed. Binding sites of the first plurality of binding sites and/or the second plurality of binding sites can be at positions that are not predetermined, can be ordered, can be irregularly distributed, and/or can be randomly distributed. A subset of binding sites can have a configuration that is not predetermined. A configuration can comprise the number binding sites in the subset of binding sites and/or positions of the binding sites in the subset of binding sites. The first subset of binding sties and the second subset of binding sites can have configurations that are different (or identical).

Also provided herein include a nucleic acid tile comprising a scaffold nucleic acid and a plurality of staple oligonucleotides. One, one or more, or each of the plurality of staple oligonucleotides can comprise two binding domains hybridized to different regions of the scaffold nucleic acid, thereby forming a double-crossover motif comprising the scaffold nucleic acid and the staple oligonucleotide. The plurality of staple oligonucleotides can comprise a first anchor oligonucleotide that protrudes from a first face of the nucleic acid tile. The nucleic acid tile can comprise a deoxyribonucleic acid (DNA). The scaffold nucleic acid can comprise a DNA. The plurality of staple oligonucleotides can comprise DNAs.

Disclosed herein include methods of forming a nucleic acid tile. In some embodiments, a method of forming a nucleic acid tile comprises providing a staple solution comprising the plurality of staple oligonucleotides. The method can comprise providing a scaffold solution comprising a scaffold nucleic acid. A staple oligonucleotide may comprise a plurality of binding domains that hybridized to different regions of the scaffold, such as at least 2, at least 3, or at least 5 binding domains. Portions of the staple oligonucleotide between binding domains may be single stranded, double stranded, and/or may comprise a non-nucleic acid polymeric spacer such as a linear or branched polymer. In certain aspects, a polymer spacer between binding domains, such as a PEG linker, may improve stability and/or protect the nucleic acid tile during sequencing. The method can comprise combining the staple solution and the scaffold solution to form a reaction solution. The method can comprise subject the reaction solution to thermal annealing, thereby forming the nucleic acid tile. The reaction solution can differ. The method can comprise purifying the nucleic acid tile from molecules of the scaffold nucleic acid and molecules of the plurality of staple oligonucleotides that are not parts of molecules of the nucleic acid tile. The method can comprise purifying the nucleic acid tile comprises size-based purification.

A method of rolling circle amplification (RCA) is contemplated herein. A method of RCA can comprise providing a nucleic acid tile. The method can comprise providing a target nucleic acid. The method can comprise circularizing the target nucleic acid using the splint sequence of the nucleic acid tile. The method can comprise performing rolling circle amplification by extending the splint sequence using the target nucleic acid as the template to generate the nucleic acid tile each comprising concatemeric copies of the target nucleic acid. A method of RCA can comprise providing a plurality of nucleic acid tiles. The method can comprise providing a plurality of target nucleic acids. The method can comprise hybridizing each of the plurality of target nucleic acids to the splint sequence of a nucleic acid tile of the plurality of nucleic acid tiles. The method can comprise circularizing the target nucleic acid hybridized to the splint sequence of the nucleic acid tile. The method can comprise performing rolling circle amplification by extending the splint sequence of the nucleic acid tile using the target nucleic acid hybridized thereto as a template to generate a nucleic acid cluster comprising the nucleic acid tile with the splint sequence thereof extended to include concatemeric copies of the target nucleic acid.

Disclosed herein include methods of aligning a plurality of flow cell images. In some embodiments, a method of aligning a plurality of flow cell images is under control of a processor and comprises: obtaining a plurality of flow cell images from a flow cell surface having a first regular binding site region and a second regular binding site region separated by an irregular binding site region. The method can comprise: aligning the irregular binding site region in the plurality of flow cell images to align the plurality of flow cell images. In some embodiments, a method of aligning a plurality of flow cell images is under control of a processor and comprises: obtaining a plurality of flow cell images from a flow cell surface having regular binding site regions separated by irregular binding site regions. The method can comprise: aligning the irregular binding site regions in the plurality of flow cell images to align the plurality of flow cell images. In some embodiments, a method of aligning a plurality of flow cell images is under control of a processor and comprises: obtaining a plurality of flow cell images from a flow cell surface comprising ordered binding site separated by disjunctions. The method can comprise: aligning the disjunctions in the plurality of flow cell images to align the plurality of flow cell images. The aligning can comprise translating one flow cell image relative to a second flow cell image. The aligning can comprise rotating one image relative to a second image.

Also provided herein are methods of sorting a plurality of flow cell images. In some embodiments, a method of sorting a plurality of flow cell images is under control of a processor and comprises: obtaining a plurality of flow cell images. The method can comprise: identifying a first regular, irregular, or random binding site region and a second regular, irregular, or random binding site region separated by an irregular or random binding site region in each of the plurality of cell images. The method can comprise: sorting the plurality of flow cell images such that flow cell images having an identical irregular or random binding site region are assigned to a common group and two flow cell images having different irregular or random binding site region are assigned to different common groups. In some embodiments, a method of sorting a plurality of flow cell images is under control of a processor and comprises: obtaining a plurality of flow cell images. The method can comprise: identifying an irregular binding site region in each of the plurality of flow cell images that separates a first regular, irregular, or random binding site region and a second regular, irregular, or random binding site region in the flow cell image. The method can comprise: sorting the plurality of flow cell images such that flow cell images having an identical irregular or random binding site region are assigned to a common group and two flow cell images having different irregular or random binding site region are assigned to different common groups.

The present disclosure provides methods of performing quality assessment on an image. In some embodiments, a method of performing quality assessment is under control of a processor and comprises: receiving an image collected from a surface comprising a plurality of binding sites. The method can comprise: identifying a first signal from a first binding site. The method can comprise: identifying a second signal from a second binding site. The method can comprise: determining a distance separating the first binding site from the second binding site. The method can comprise: negatively assessing the image if the distance is below a threshold.

Disclosed herein include methods of orienting a plurality of flow cell images. In some embodiments, a method of orienting a plurality of flow cell images is under control of a processor and comprises: identifying an irregular or random colony region of a plurality of colonies common to a plurality of the flow cell images. The method can comprise: orienting one or more of the plurality of flow cell images such that the irregular or random colony region is aligned among the plurality of flow cell images.

Disclosed herein include flow cell imaging systems. In some embodiments, a flow cell imaging system comprises: a flow cell having distributed thereon a plurality of binding sites, at least some of the binding sites are randomly distributed. The flow cell imaging system can comprise: an excitation source to excite fluorophores at the binding sites. The flow cell imaging system can comprise: an image digitization interface comprising a plurality of pixels.

### Specifying Binding Sites on a Planar Structure

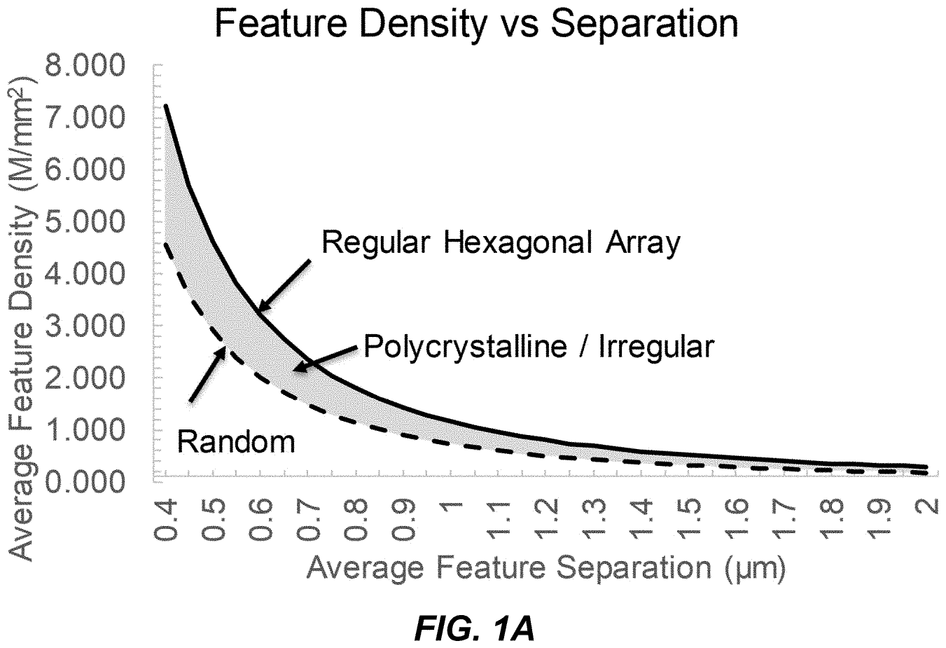

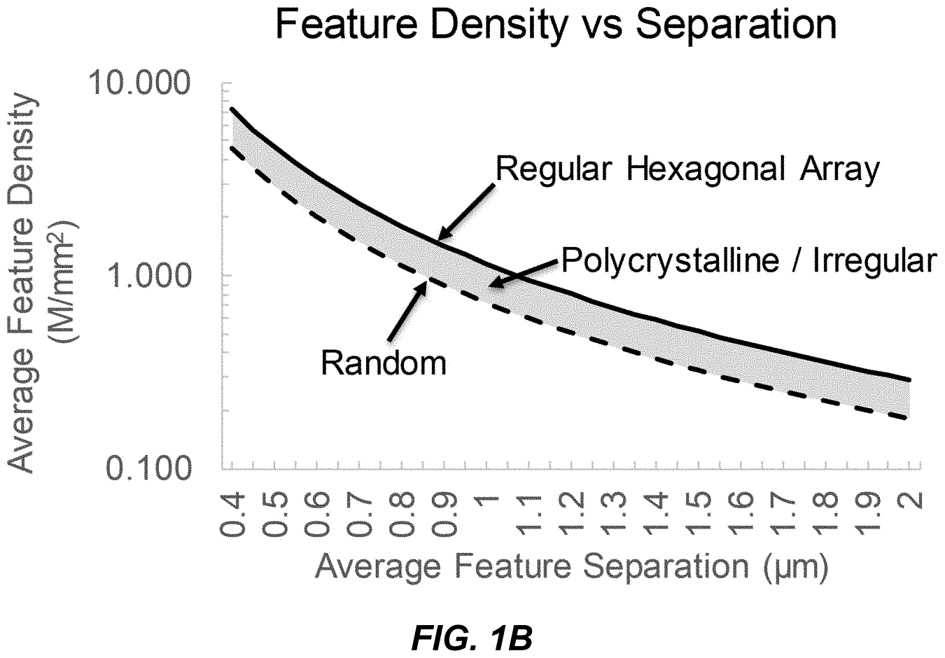

Flow cells with surfaces having discrete spots (or sites or pads) for colony formation are used for next generation sequencing. There is an inverse relationship between the density of the spots and the average distance between the spots (FIGS. 1A-1B). For any given spot density, ordered spots (such as spots of regular hexagonal array) have a shorter average distance between the spots as compared to random spots. For any given spot density, irregular spots (also referred to herein as polycrystalline spots) have a shorter average distance between the spots as compared to random spots and a longer average distance between the spots as compared to ordered spots. Making such flow cells with surfaces having predetermined and ordered spots or top-down lithography can be expensive and time consuming; though the flow cells so generated have surfaces with higher spot densities (FIGS. 1A-1B, top curves). In contrast, flow cells with surfaces having non-predetermined and random spots can be cheaper to make; though the flow cells so generated have surfaces with lower spot densities. In FIGS. 1A-1B, the curve showing the random density was generated with the assumption that the spots (or features on the spots) exclude each other.

Some embodiments of the present disclosure provide methods of generating flow cells using colloidal self-assembly (bottom-up). Such flow cells can have surfaces with ordered spots (which can enable higher spot density while maintaining good sequencing data quality) generated in a non-predetermined manner (which can enable such flow cells to be manufactured at lower costs). Alternatively or additionally, such flow cells can have surfaces with irregular spots (which can enable higher spot density while maintaining good sequencing data quality) generated in a non-predetermined manner (which can enable such flow cells to be manufactured at lower costs). A self-assembled layer of beads can be used as a mask for etching to produce closely packed spots (or sites or pads) with high densities. For example, the closely packed spots can have (or can be described using or as having) a hexagonal configuration. The spots can be aminosilane pads for capture of emPCR beads. The methods can generate structured flow cell surfaces (or planar structure surfaces) with ordered or irregularly distributed (or distributed with non-regular spacing) clusters of spots. Using the flow cell surfaces generated for next generation sequencing can result in increased surface density of good spots and improved spot identification at ultra-high density. The size of the spots, the pitch between neighbor spots, and the minimum distance between the spots can be affected by the etching process and the size and material of the beads.

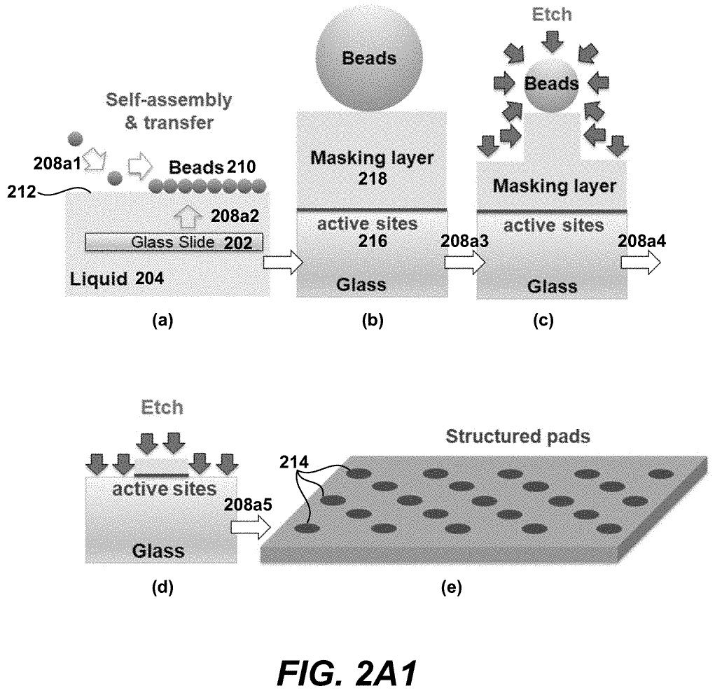

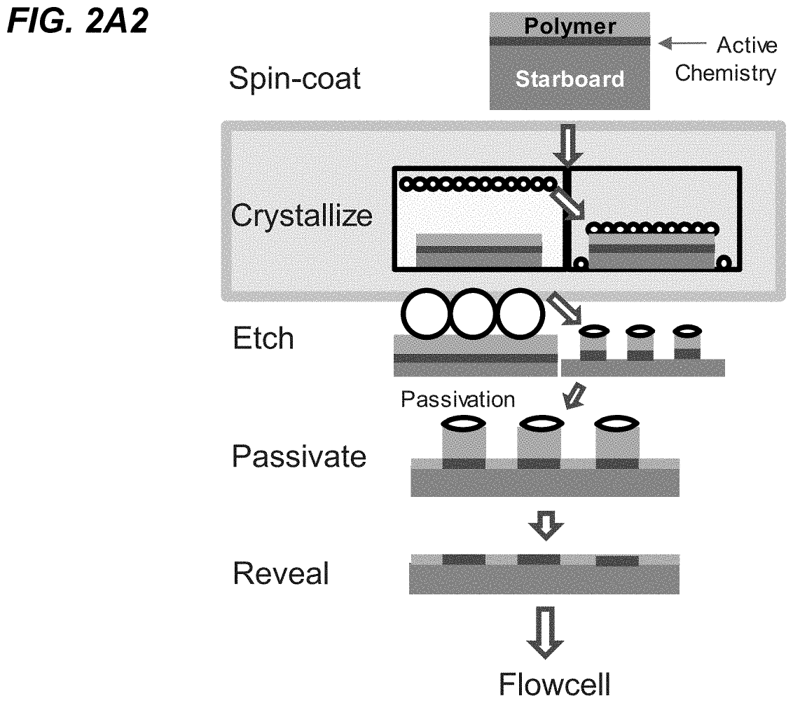

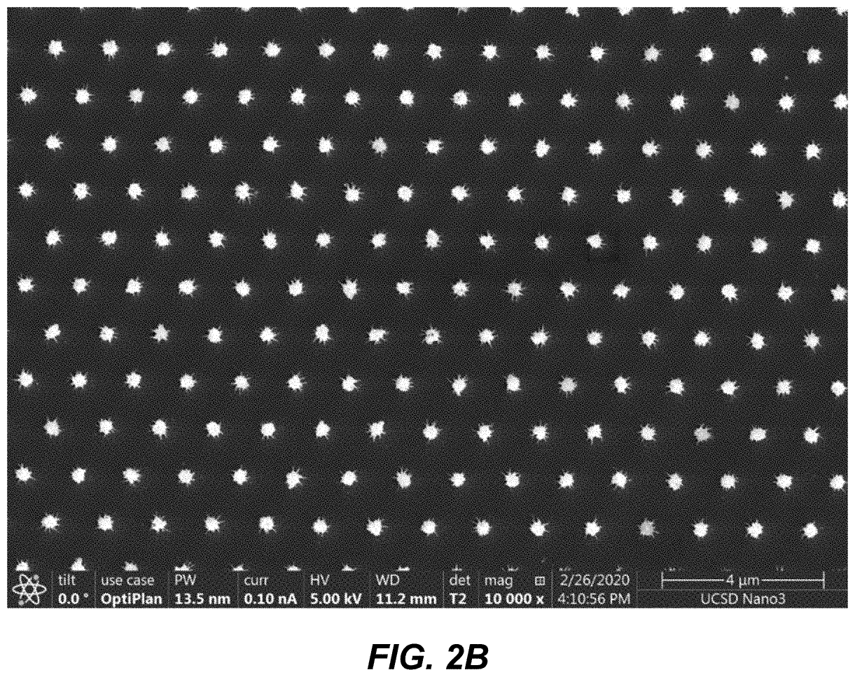

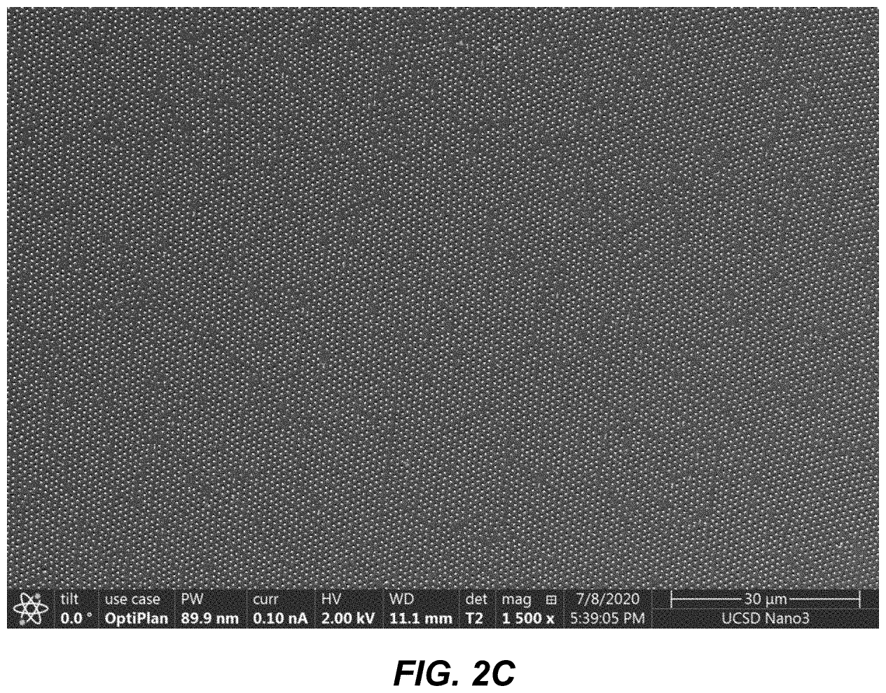

Disclosed herein include methods of specifying binding sites (also referred to herein as binding spots or active sites) on a planar (or substantially planar) structure (or substrate or flow cell surface). The binding sites can be ordered, irregularly distributed, or randomly distributed. The binding sites can be used for, for example, generating colonies for next generation sequencing. The methods of specifying binding sites on a planar structure of the present disclosure are also referred to as colloidal self-assembly (bottom-up). FIGS. 2A1-2A2 show non-limiting exemplary methods specifying binding sites on a planar structure. Referring to FIG. 2A1, a method of specifying binding sites on a planar structure can include providing a planar structure 202 (a glass slide is shown in FIG. 2A1) subsumed in a liquid 204 (FIG. 2A1, panel a). At action 208al, the method can include delivering a plurality of particles 210 (beads shown in FIG. 2A1) to a surface 212 of the liquid 204. The surface of the liquid can be the top surface of the liquid which is in contact with another medium, such as air. A percentage of the plurality of particles (e.g., 5%, 10%, 20%, 30%, 40%, 50%, 60%, 70%, 80%, 90%, or more) can self-assemble into closely packed, ordered or irregular particles. At action 208a2, the method can include removing the liquid 204 between the particles 210 and the planar structure 202, such that the plurality of particles 210 comes into contact with the planar structure 202 (FIG. 2A1, panel b). The plurality of particles 210 on the surface 212 of the liquid 204 can specify a plurality of binding sites 214 (e.g., a plurality of sites for colony formation) on the planar structure 202. The plurality of particles 208 in contact with the planar structure 202 can specify a plurality of binding sites 214 on the planar structure 202. FIG. 2A1 shows a non-limiting exemplary method of surface structuring for high-density spots (or sites) arrayed in a non-predetermined manner. FIGS. 2B and 2C show a non-limiting exemplary scanning electron microscope (SEM) images of structured surfaces (e.g., flow cell surfaces) having spots (or sites) at a high density, where the spots were generated using colloidal self-assembly in a non-predetermined manner.

A method of specifying binding sites (also referred to herein as binding spots or active sites) on planar structures can include providing a plurality of planar structure (a planar structure which is a glass slide 202 is shown in FIG. 2A1) subsumed in a liquid 204. The method can include: delivering a plurality of particles 210 (beads are shown in FIG. 2A1) to a surface 212 of the liquid 204 at action 208al. The surface of the liquid can be the top surface of the liquid which is in contact with another medium, such as air. The method can include removing the liquid 204 between the particles 210 and the plurality of planar structures at action 208a2, such that the plurality of particles 210 comes into contact with the plurality of planar structures (FIG. 2A1, panel b). The plurality of particles 210 on the surface 212 and/or in contact with the plurality of planar structures can specify a plurality of binding sites 214 on each of the plurality of planar structures. The pluralities of binding sites (the relative and/or the absolute positions or locations of the binding sites) on any two planar structures can be different. The method generates the pluralities of binding sites 214 using colloquial self-assembly of the plurality of particles 210 on the surface 212 of the liquid 204 such that the pluralities of binding sites on any two planar structures can be different. In contrast, top-down lithography uses a lithography mask to generate flow cells with surfaces having identical binding sites.

A method of specifying binding sites (also referred to herein as binding spots or active sites) on a planar structure 202 (a glass slide is shown in FIG. 2A1) can include providing a planar structure 202 having deposited thereon an active site layer (or a substance layer or a binding site layer) 216 and a masking layer 218 (FIG. 2A1, panel b). The method can include depositing a plurality of particles 210 (beads are shown in FIG. 2A1) onto the masking layer 218 of the planar structure 202. The method can include exposing the planar structure 202 to an etching agent at action 208a3 so as to differentially remove the masking layer 218 from regions not shielded by the plurality of beads 210 (FIG. 2A, panel c). The method can include removing the masking layer 218 and the active site layer 216 from regions not shielded from the etching layer by the plurality of beads at action 208a3 (FIG. 2A1, panel c). The method can include removing remaining masking layer from regions shielded by the plurality of beads at action 208a4 with the active site layer at the binding sites remaining (FIG. 2A1, panel d). The method thereby specifies a plurality of binding sites 214 comprising the active site layer remaining at action 208a5 (FIG. 2A1, panel e).

A method of specifying binding sites (also referred to herein as binding spots or active sites) on a planar structure can include providing a planar structure 202 (a glass slide is shown in FIG. 2A1) having deposited thereon an active site layer 216 and a masking layer 28 (FIG. 2A1, panel a). The method can include depositing a plurality of particles 210 (beads are shown in FIG. 2A) onto the masking layer 218 of the planar structure 202 at action 208al. The method can include exposing the planar structure 202 to an etching agent at action 208a3, thereby: removing the masking layer and the active site layer from regions not shielded from the etching layer by the plurality of beads (FIG. 2A, panel c). The method can include removing remaining masking layer from regions shielded by the plurality of beads at action 208a4 with the active site layer at the binding sites remaining (FIG. 2A1, panel d). The method can include removing particle material and the plurality of particles, or any portion of each particle remaining. The method thereby specifies a plurality of binding sites 214 comprising the active site layer remaining at action 208a5 (FIG. 2A, panel e).

The planar structure, the active site layer, and/or the masking layer is often uniform (or substantially uniform) prior to any step of the method, such as the depositing the plurality of beads. The masking layer can be uniform prior to the depositing the plurality of beads. The active site layer can be uniform prior to the depositing the plurality of beads. The planar structure can be uniform prior to the depositing the plurality of beads. That is, through the disclosure herein beads can be deposited onto a planar surface lacking patterning that would otherwise dictate a pre-determined array of binding site positions. Binding sites can be arranged into locally regular distributions such as those described above and elsewhere herein, but the locally regular distributions do not arise from predetermined patterns on the surface.

### Delivering Particles

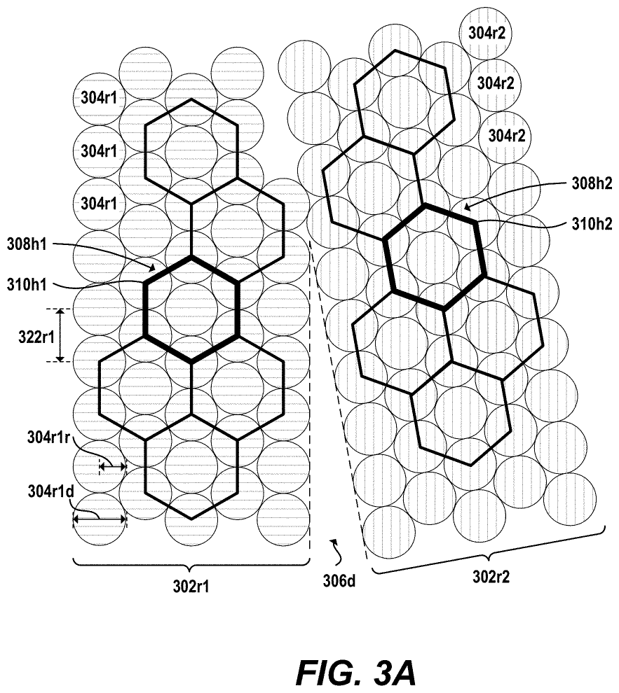

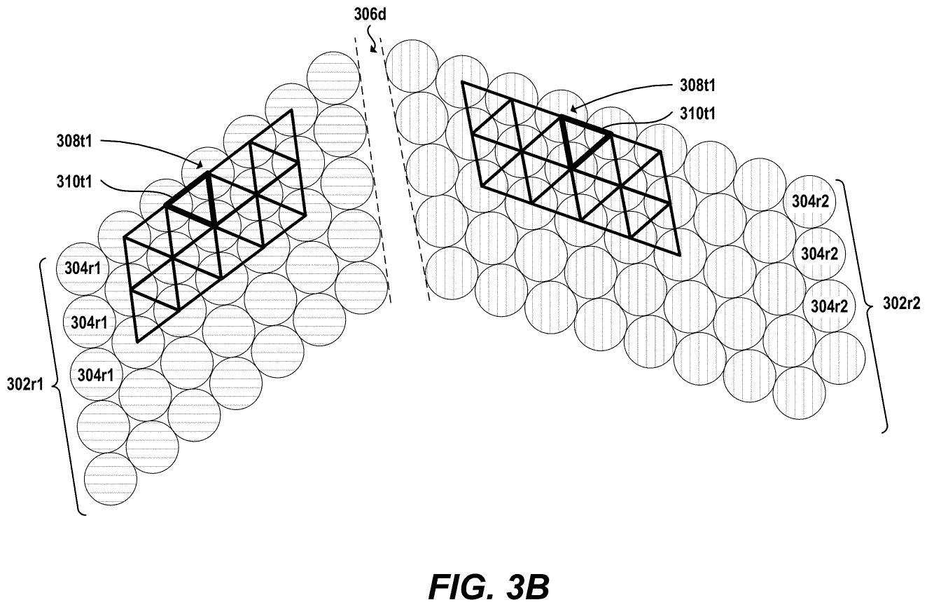

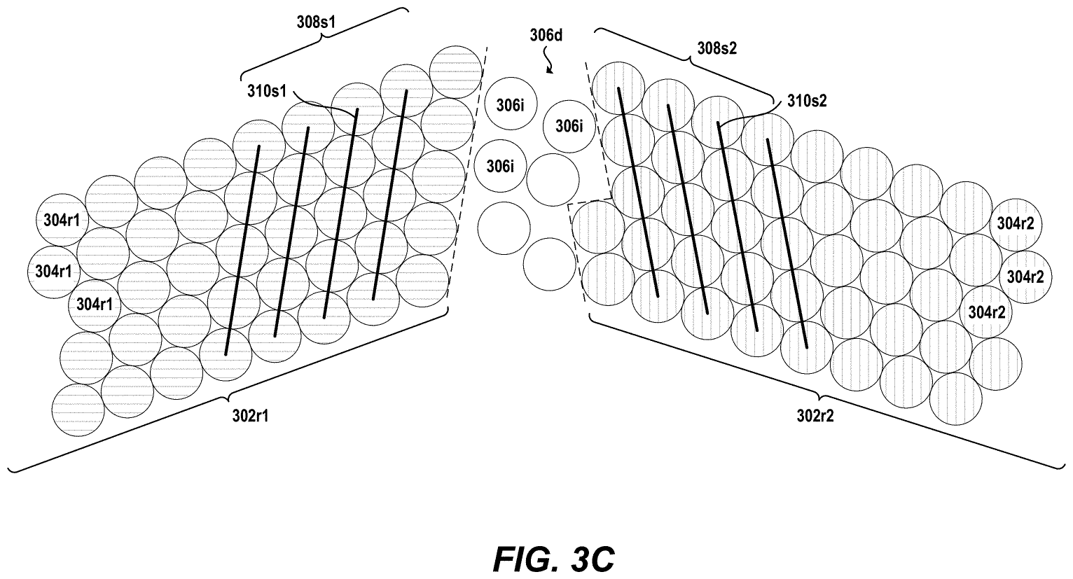

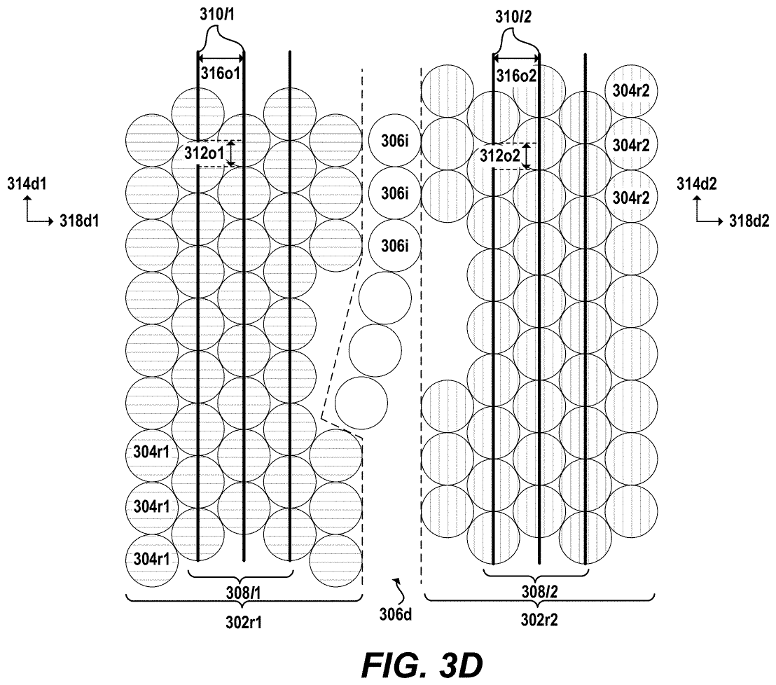

The particles can be delivered to the surface of the liquid prior to the particles come into contact with the planar structure. Referring to FIG. 2A1, delivering the plurality of particles 210 to the surface 212 of the liquid 204 at action 208al can include locally saturating (or partially saturating) the surface 212 of the liquid 204 with some or all of the plurality of particles. Referring to FIGS. 3A-3D, locally saturating (or partially saturating) the surface of the liquid with some or all of the plurality of particles can result in a first crystal lattice 302r1 being formed at the surface. The first crystal lattice 302r1 can comprise a first subset of first particles 304r1 of the plurality of particles. The first particles 304r1 can be tightly packed and ordered (or regularly ordered); though its size and position on the substrate surface is not predetermined. Locally saturating (or partially saturating) the surface of the liquid with some or all of the plurality of particles can result in a second crystal lattice 302r2 being formed at the surface. The second crystal lattice 302r2 can comprise a second subset of second particles 304r2 of the plurality of particles. The second particles 304r2 can be tightly packed and ordered; though its size and position on the substrate surface is not predetermined. The second crystal lattice 302r2 can be separated from the first crystal lattice by a disjunction or crack 306d. The disjunction 306d can include no particles as shown in FIGS. 3A and 3B, or can include non-ordered (or randomly distributed, or disordered) particles 306i as shown in FIGS. 3C and 3D. The size and location of the disjunction 306d and the presence or absence of non-ordered particles 306i in the disjunction is not predetermined. The first crystal lattice 302r1 and the second crystal lattice 302r2 have different orientations such that the two crystal lattices (or a subset of particles of the two crystal lattices) are related to each other by a rotation (and a translation). A crystal lattice can also be referred to as a local crystal lattice.

Depositing a plurality of beads often comprises packing beads of the plurality of beads into a configuration comprising a first local crystal lattice (or a first irregular array) and a second local crystal lattice (or a second irregular array) separated by a local disjunction. In some embodiments, delivering a plurality of particles a surface of a liquid can include locally saturating (or partially saturating) the surface of the liquid with some or all of the plurality of particles. Locally saturating (or partially saturating) the surface of the liquid with some or all of the plurality of particles can result in a first irregular array being formed at the surface. Locally saturating (or partially saturating) the surface of the liquid with some or all of the plurality of particles can result in a second irregular array being formed at the surface.

In some embodiments, delivering the plurality of particles comprises delivering a first subset of particles of the plurality of particles to a first location of the surface of the liquid and a second subset of particles of the plurality of particles to a second location of the surface of the liquid simultaneously and/or sequentially. Delivering the plurality of particles can comprise delivering a plurality of subsets (e.g., 2, 3, 4, 5, 6, 7, 8, 9, 10, or more subsets) of particles of the plurality of particles to different locations at the surface of the liquid simultaneously and/or sequentially.

### Liquid

A number of liquids are consistent with the disclosure herein. For example, the liquid with a density higher than the density of the particles can be used such that the particles are at the top surface of the liquid after the particles are introduced into the liquid. Some suitable liquids have one or more of the following traits: a surface tension suitable for the packing of particles, a viscosity sufficient to allow removal of the liquid from between the particles and the planar surface without substantial disruption of particle configuration, and a volatility suitable for evaporation of the liquid so as to deposit the particles on the planar surface without substantial disruption of particle configuration. The liquid is often hydrophilic, although other liquids are consistent with some embodiments. The liquid with the desired properties, such as density, viscosity and surface tension, can be used. The liquid can comprise a buffer solution, a salt solution, water, an organic solvent, a polar solvent, a non-polar solvent, an oil, a natural oil, a synthetic oil, an organic oil, a mineral oil, a paraffin oil, a hydrocarbon oil, a non-hydrocarbon oil, a silicone oil, a volatile liquid, or a combination thereof. In certain embodiments of the methods provided herein, the carrier oil is a natural oil, a synthetic oil, or a combination thereof. The oil can be a hydrocarbon oil or a non-hydrocarbon oil. The oil can be a mineral oil (an example of a natural, hydrocarbon oil having from about 10 to about 60 carbon atoms). The oil can be silicone oil (an example of a synthetic, non-hydrocarbon oil). The oil can be a synthetic oil, an organic oil, a mineral oil, a paraffin oil, paraffin such as liquid paraffin, a fatty acid (for example stearic acid) an alkanes mixture, a pure alkane (such as decane, undecane, dodecane, tridecane, tetradecane, pentadecane, hexadecane, heptadecane, octadecane, nonadacane, eicosane or heneicosane). The liquid can be of varying purity. The liquid can be of relatively high purity. The liquid can be of less than relatively high purity.

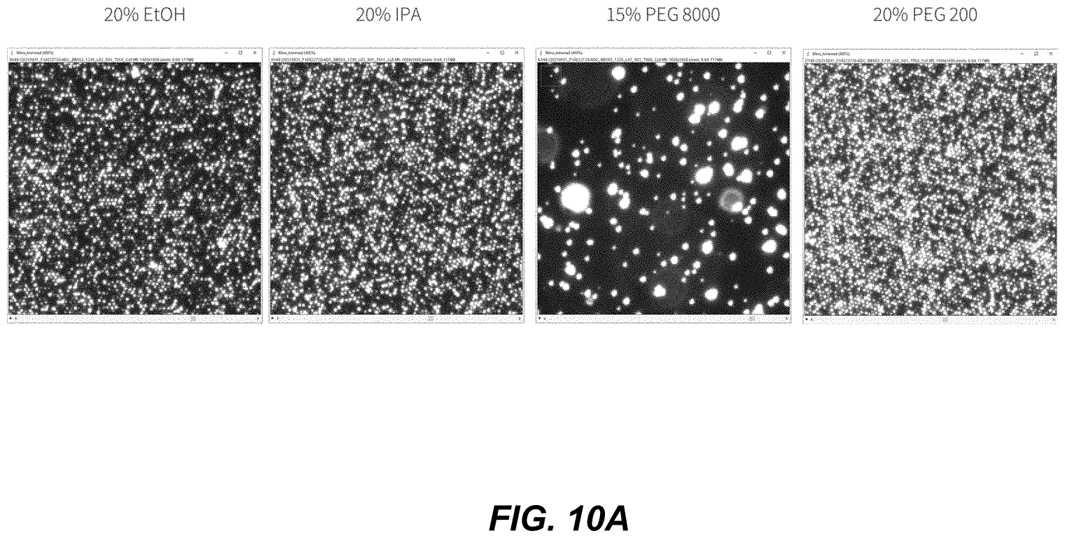

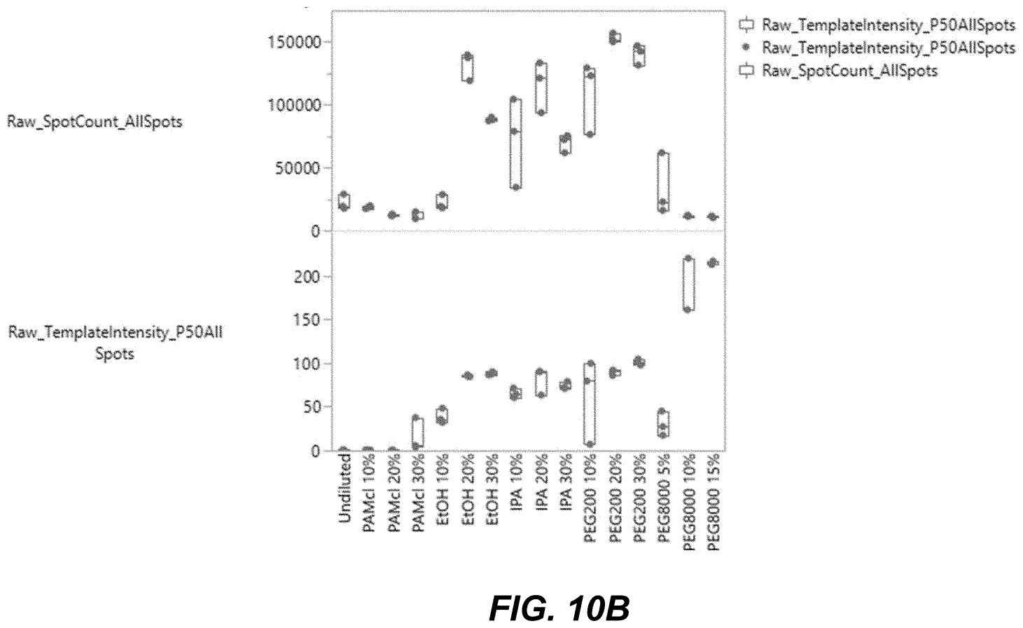

In some embodiments, the liquid comprises a spreading agent, a contaminant, or a combination thereof. The spreading agent can comprise an alcohol, such as methanol, ethanol, propyl alcohol, isopropyl alcohol (isopropanol), butyl alcohol, isobutyl alcohol (isobutanol), pentyl alcohol, pentanol (amyl alcohol), hexyl alcohol, heptyl alcohol, octyl alcohol, nonyl alcohol, and decyl alcohol. The contaminant can comprise a surfactant, a crowding agent, sucrose, urea, a polyacrylic acid, pyridine aldoxime methyl chloride, or a combination thereof. The surfactant can comprise sodium dodecyl sulfate, Tween, or a combination thereof. The surfactant can be an anionic surfactant (e.g., docusate (dioctyl sodium sulfosuccinate), perfluorooctanesulfonate (PFOS), perfluorobutanesulfonate, alkyl-aryl ether phosphate, and alkyl ether phosphate), a cationic surfactant (e.g., octenidine dihydrochloride, cetrimonium bromide (CTAB), cetylpyridinium chloride (CPC), benzalkonium chloride (BAC), benzethonium chloride (BZT), dimethyldioctadecylammonium chloride, and dioctadecyldimethylammonium bromide (DODAB)), a zwitterionic surfactant (e.g., a phospholipid, phosphatidylserine, phosphatidylethanolamine, phosphatidylcholine, and sphingomyelin), or a non-ionic surfactant. A non-ionic surfactant can be a fatty alcohol ethoxylate (e.g., narrow-range ethoxylate, octaethylene glycol monododecyl ether, or pentaethylene glycol monododecyl ether), a alkylphenol ethoxylate (e.g., nonoxynols, Triton X-100), a fatty acid ethoxylate, an ethoxylated amines and/or fatty acid amide (e.g., polyethoxylated tallow amine, cocamide monoethanolamine, cocamide diethanolamine), a terminally blocked ethoxylate (e.g., poloxamer), a fatty acid ester of polyhydroxy compound, a fatty acid esters of glycerol (e.g., glycerol monostearate, glycerol monolaurate), a fatty acid ester of sorbitol (e.g., Span, such as sorbitan monolaurate, sorbitan monostearate, sorbitan tristearate, or Tween, such as Tween 20, Tween 40, Tween 60, Tween 80) a fatty acid esters of sucrose, an alkyl polyglucoside (e.g., decyl glucoside, lauryl glucoside, octyl glucoside). The crowing agent can comprise a polyethylene glycol (PEG). The PEG can comprise a PEG with an average molecular of about 200 daltons (e.g., PEG 200) to about 8000 (e.g., PEG 8000), such as 200 daltons, 300 daltons, 800 daltons, 1000 daltons, 1500 daltons, 2000 daltons, 3000 daltons, 4000 daltons, 6000 daltons, or 8000 daltons. The concentration of a spreading agent (or a contaminant) can have a concentration from about 1% to about 20%. For example, the concentration can be, be about, be at least, be at least about, be at most, or be at most about 1%, 2%, 3%, 4%, 5%, 6%, 7%, 8%, 9%, 10%, 11%, 12%, 13%, 14%, 15%, 16%, 17%, 18%, 19%, 20% or a number or a range between any two of these values. With the liquid comprising one or more spreading agent and/or one or more contaminants, the resultant binding sites can be irregularly distributed (see FIGS. 10A-10B). Smaller hydrophilic polymers (such as PEG 200) may be useful for RCA products deposition FIGS. 10A-10B show that various additives can be used for improving deposition of RCA products on structured (regular or irregular) surface. Deposition of RCA products on an unstructured amino silane surface can be accomplished without additives in the deposition buffer. The deposition buffer can be the same buffer in which the RCA products are made. This deposition buffer is not compatible with deposition on the structured surface. Multiple additives have been identified for increasing deposition efficiency on the structured surface. Alcohols (such as ethanol and isopropanol) and crowding agents (such as PEG 200 and PEG 8000) have been shown to increase deposition efficiency. FIG. 10B shows spot counts and spot intensities of deposited clusters. Higher and tight distributions of spot count and intensity are preferred.

The liquid can be selected to have a desired density. The density of the liquid can be, be about, be at least, be at least about, be at most, or be at most about, 0.01 g/cm³, 0.02 g/cm³, 0.03 g/cm³, 0.04 g/cm³, 0.05 g/cm³, 0.06 g/cm³, 0.07 g/cm³, 0.08 g/cm³, 0.09 g/cm³, 0.1 g/cm³, 0.2 g/cm³, 0.3 g/cm³, 0.4 g/cm³, 0.5 g/cm³, 0.6 g/cm³, 0.7 g/cm³, 0.8 g/cm³, 0.9 g/cm³, 1 g/cm³, 1.1 g/cm³, 1.2 g/cm³, 1.3 g/cm³, 1.4 g/cm³, 1.5 g/cm³, 1.6 g/cm³, 1.7 g/cm³, 1.8 g/cm³, 1.9 g/cm³, 2 g/cm³, 2.1 g/cm³, 2.2 g/cm³, 2.3 g/cm³, 2.4 g/cm³, 2.5 g/cm³, 2.6 g/cm³, 2.7 g/cm³, 2.8 g/cm³, 2.9 g/cm³, 3 g/cm³, 3.1 g/cm³, 3.2 g/cm³, 3.3 g/cm³, 3.4 g/cm³, 3.5 g/cm³, 3.6 g/cm³, 3.7 g/cm³, 3.8 g/cm³, 3.9 g/cm³, 4 g/cm³, 4.1 g/cm³, 4.2 g/cm³, 4.3 g/cm³, 4.4 g/cm³, 4.5 g/cm³, 4.6 g/cm³, 4.7 g/cm³, 4.8 g/cm³, 4.9 g/cm³, 5 g/cm³, 5.1 g/cm³, 5.2 g/cm³, 5.3 g/cm³, 5.4 g/cm³, 5.5 g/cm³, 5.6 g/cm³, 5.7 g/cm³, 5.8 g/cm³, 5.9 g/cm³, 6 g/cm³, 6.1 g/cm³, 6.2 g/cm³, 6.3 g/cm³, 6.4 g/cm³, 6.5 g/cm³, 6.6 g/cm³, 6.7 g/cm³, 6.8 g/cm³, 6.9 g/cm³, 7 g/cm³, 7.1 g/cm³, 7.2 g/cm³, 7.3 g/cm³, 7.4 g/cm³, 7.5 g/cm³, 7.6 g/cm³, 7.7 g/cm³, 7.8 g/cm³, 7.9 g/cm³, 8 g/cm³, 8.1 g/cm³, 8.2 g/cm³, 8.3 g/cm³, 8.4 g/cm³, 8.5 g/cm³, 8.6 g/cm³, 8.7 g/cm³, 8.8 g/cm³, 8.9 g/cm³, 9 g/cm³, 9.1 g/cm³, 9.2 g/cm³, 9.3 g/cm³, 9.4 g/cm³, 9.5 g/cm³, 9.6 g/cm³, 9.7 g/cm³, 9.8 g/cm³, 9.9 g/cm³, 10 g/cm³, 11 g/cm³, 12 g/cm³, 13 g/cm³, 14 g/cm³, 15 g/cm³, 16 g/cm³, 17 g/cm³, 18 g/cm³, 19 g/cm³, 20 g/cm³, or a number or a range between any two of these values. For example, the density of the liquid can be about 0.1 g/cm³ to about 10 g/cm³. The density of the liquid can be higher than a density of one, one or more, or each, of the plurality of particles such that the particle floats at the surface of liquid.

The viscosity of the liquid can vary. The viscosity of the liquid can be, be about, be at least, be at least about, be at most, or be at most about, 0.1 mPa·s, 0.2 mPa·s, 0.3 mPa·s, 0.4 mPa·s, 0.5 mPa·s, 0.6 mPa·s, 0.7 mPa·s, 0.8 mPa·s, 0.9 mPa·s, 1 mPa·s, 2 mPa·s, 3 mPa·s, 4 mPa·s, 5 mPa·s, 6 mPa·s, 7 mPa·s, 8 mPa·s, 9 mPa·s, 10 mPa·s, 20 mPa·s, 30 mPa·s, 40 mPa·s, 50 mPa·s, 60 mPa·s, 70 mPa·s, 80 mPa·s, 90 mPa·s, 100 mPa·s, 200 mPa·s, 300 mPa·s, 400 mPa·s, 500 mPa·s, 600 mPa·s, 700 mPa·s, 800 mPa·s, 900 mPa·s, 1000 mPa·s, 2,000 mPa·s, 3,000 mPa·s, 4,000 mPa·s, 5,000 mPa·s, 6,000 mPa·s, 7,000 mPa·s, 8,000 mPa·s, 9,000 mPa·s, 10,000 mPa·s, 20,000 mPa·s, 30,000 mPa·s, 40,000 mPa·s, 50,000 mPa·s, 60,000 mPa·s, 70,000 mPa·s, 80,000 mPa·s, 90,000 mPa·s, 100,000 mPa·s, 200,000 mPa·s, 300,000 mPa·s, 400,000 mPa·s, 500,000 mPa·s, 600,000 mPa·s, 700,000 mPa·s, 800,000 mPa·s, 900,000 mPa·s, 1,000,000 mPa·s, 2,000,000 mPa·s, 3,000,000 mPa·s, 4,000,000 mPa·s, 5,000,000 mPa·s, 6,000,000 mPa·s, 7,000,000 mPa·s, 8,000,000 mPa·s, 9,000,000 mPa·s, 10,000,000 mPa·s, 20,000,000 mPa·s, 30,000,000 mPa·s, 40,000,000 mPa·s, 50,000,000 mPa·s, 60,000,000 mPa·s, 70,000,000 mPa·s, 80,000,000 mPa·s, 90,000,000 mPa·s, 100,000,000 mPa·s, or a number or a range between any two of these values. For example, a viscosity of the liquid is about 0.1 millipascal-second (mPa·s) to about 10,000,000 mPa·s. The viscosity of the liquid can be, be about, be at least, be at least about, be at most, or be at most about 10⁻¹ centipoise (cP), 1 cP, 10 cP, 10² cP, 10³ cP, 10⁴ cP, 10⁵ cP, 10⁶ cP, 10⁷ cP, 10⁸ cP, or a number or a range between any two of these values

The liquid can be selected with a desired surface tension (and thus evaporation rate). The surface tension of the liquid can be, be about, be at least, be at least about, be at most, or be at most about, 1 mN·m⁻¹, 2 mN·m⁻¹, 3 mN·m⁻¹, 4 mN·m⁻¹, 5 mN·m⁻¹, 6 mN·m⁻¹, 7 mN·m⁻¹, 8 mN·m⁻¹, 9 mN·m⁻¹, 10 mN·m⁻¹, 11 mN·m⁻¹, 12 mN·m⁻¹, 13 mN·m⁻¹, 14 mN·m⁻¹, 15 mN·m⁻¹, 16 mN·m⁻¹, 17 mN·m⁻¹, 18 mN·m⁻¹, 19 mN·m⁻¹, 20 mN·m⁻¹, 21 mN·m⁻¹, 22 mN·m⁻¹, 23 mN·m⁻¹, 24 mN·m⁻¹, 25 mN·m⁻¹, 26 mN·m⁻¹, 27 mN·m⁻¹, 28 mN·m⁻¹, 29 mN·m⁻¹, 30 mN·m⁻¹, 31 mN·m⁻¹, 32 mN·m⁻¹, 33 mN·m⁻¹, 34 mN·m⁻¹, 35 mN·m⁻¹, 36 mN·m⁻¹, 37 mN·m⁻¹, 38 mN·m⁻¹, 39 mN·m⁻¹, 40 mN·m⁻¹, 41 mN·m⁻¹, 42 mN·m⁻¹, 43 mN·m⁻¹, 44 mN·m⁻¹, 45 mN·m⁻¹, 46 mN·m⁻¹, 47 mN·m⁻¹, 48 mN·m⁻¹, 49 mN·m⁻¹, 50 mN·m⁻¹, 51 mN·m⁻¹, 52 mN·m⁻¹, 53 mN·m⁻¹, 54 mN·m⁻¹, 55 mN·m⁻¹, 56 mN·m⁻¹, 57 mN·m⁻¹, 58 mN·m⁻¹, 59 mN·m⁻¹, 60 mN·m⁻¹, 61 mN·m⁻¹, 62 mN·m⁻¹, 63 mN·m⁻¹, 64 mN·m⁻¹, 65 mN·m⁻¹, 66 mN·m⁻¹, 67 mN·m⁻¹, 68 mN·m⁻¹, 69 mN·m⁻¹, 70 mN·m⁻¹, 71 mN·m⁻¹, 72 mN·m⁻¹, 73 mN·m⁻¹, 74 mN·m⁻¹, 75 mN·m⁻¹, 76 mN·m⁻¹, 77 mN·m⁻¹, 78 mN·m⁻¹, 79 mN·m⁻¹, 80 mN·m⁻¹, 81 mN·m⁻¹, 82 mN·m⁻¹, 83 mN·m⁻¹, 84 mN·m⁻¹, 85 mN·m⁻¹, 86 mN·m⁻¹, 87 mN·m⁻¹, 88 mN·m⁻¹, 89 mN·m⁻¹, 90 mN·m⁻¹, 91 mN·m⁻¹, 92 mN·m⁻¹, 93 mN·m⁻¹, 94 mN·m⁻¹, 95 mN·m⁻¹, 96 mN·m⁻¹, 97 mN·m⁻¹, 98 mN·m⁻¹, 99 mN·m⁻¹, 100 mN·m⁻¹, 110 mN·m⁻¹, 120 mN·m⁻¹, 130 mN·m⁻¹, 140 mN·m⁻¹, 150 mN·m⁻¹, 160 mN·m⁻¹, 170 mN·m⁻¹, 180 mN·m⁻¹, 190 mN·m⁻¹, 200 mN·m⁻¹, 210 mN·m⁻¹, 220 mN·m⁻¹, 230 mN·m⁻¹, 240 mN·m⁻¹, 250 mN·m⁻¹, 260 mN·m⁻¹, 270 mN·m⁻¹, 280 mN·m⁻¹, 290 mN·m⁻¹, 300 mN·m⁻¹, 310 mN·m⁻¹, 320 mN·m⁻¹, 330 mN·m⁻¹, 340 mN·m⁻¹, 350 mN·m⁻¹, 360 mN·m⁻¹, 370 mN·m⁻¹, 380 mN·m⁻¹, 390 mN·m⁻¹, 400 mN·m⁻¹, 410 mN·m⁻¹, 420 mN·m⁻¹, 430 mN·m⁻¹, 440 mN·m⁻¹, 450 mN·m⁻¹, 460 mN·m⁻¹, 470 mN·m⁻¹, 480 mN·m⁻¹, 490 mN·m⁻¹, 500 mN·m⁻¹, 550 mN·m⁻¹, 600 mN·m⁻¹, 650 mN·m⁻¹, 700 mN·m⁻¹, 750 mN·m⁻¹, 800 mN·m⁻¹, 850 mN·m⁻¹, 900 mN·m⁻¹, 950 mN·m⁻¹, 1000 mN·m⁻¹, or a number or a range between any two of these values. For example, the surface tension of the liquid is about 10 mN·m⁻¹ to about 500 mN·m⁻¹.

### Particles/Beads

The plurality of particles (such as beads) in contact with the planar structure can be on (or on top of) the planar structure. FIG. 2A1, panel b shows a particle 210 of a plurality of particles being on the planar structure 202. Particles can have the same or different properties and/or materials. For example, two (or more, such as 1%, 2%, 3%, 4%, 5%, 6%, 7%, 8%, 9%, 10%, 20%, 30%, 40%, 50%, 70%, 80%, 90%, 99%, or more, of the plurality of particles) particles can have an identical material. Every particle can have an identical material. Two (or more, such as 1%, 2%, 3%, 4%, 5%, 6%, 7%, 8%, 9%, 10%, 20%, 30%, 40%, 50%, 70%, 80%, 90%, 99%, or more, of the plurality of particles) particles can have different materials. The plurality of particles can comprise two subsets of particles having different materials. Each subset can include 1 particle, 2 parties or more particles. Each subset can include 1%, 2%, 3%, 4%, 5%, 6%, 7%, 8%, 9%, 10%, 20%, 30%, 40%, 50%, 70%, 80%, 90%, 99%, or more, of the plurality of particles).

A multitude of particle materials are consistent with the present disclosure. For example, the material of one, one or more, or each, of the plurality of particles comprises glass, agarose, gelatin, hydrogel, ceramic, plastic, acrylic polymer, metal, latex, cellulose, nylon, silicone, or a combination thereof. The particles can be magnetic. For example, the material of one, one or more, or each, of the plurality of particles comprises polydimethylsiloxane (PDMS), polyethylene terephthalate (PET), polybutylene terephthalate (PBT), polymethyl methacrylate (PMMA), polyethylene, polymethylene, polypropylene (PP), polystyrene (PS), poly(vinyl acetate), polyurethane, or a combination thereof. For instance, the material of one particle of the plurality of particles comprises nanomaterials (e.g., conductive nanomaterials). Exemplary nanomaterials include, but are not limited to, aluminum nanomaterial, carbon nanomaterial, cobalt carbon coated nanomaterial, copper nanomaterial, copper nanomaterial, copper-zinc alloy nanomaterial, diamond nanomaterial, gold nanomaterial, iron nanomaterial, iron-nickel alloy nanomaterial, molybdenum nanomaterial, magnesium nanomaterial, nickel nanomaterial, palladium nanomaterial, platinum nanomaterial, silver nanomaterial, silver-copper alloy nanomaterial, tantalum nanomaterial, tin nanomaterial, indium doped tin oxide nanomaterial, titanium nanomaterial, titanium nitride nanomaterial, tungsten nanomaterial, zinc nanomaterial, calcium oxide nanomaterial, hydroxyapatite nanomaterial, indium nanomaterial, silica nanomaterial, silicon nanomaterial, silicon dioxide nanomaterial, silicon nitride nanomaterial, silicon carbide nanomaterial, and the like. Exemplary nanomaterials can also include polymers such as polyethylene, polymethylene, polypropylene, or polystyrene, wherein the polymer is not covalently conjugated to components of the analyte sensor.

The material of the particle can comprise a polyolefin. The polyolefin can be a homopolymer (derived from a single monomer constituent) or a heteropolymer (derived from more than one monomer constituent), and can be either linear or branched. If the polyolefin is a heteropolymer derived from two (or more) monomer constituents, the polyolefin can assume any copolymer chain arrangement including those of a block copolymer or a random copolymer. As examples, the polyolefin can be polyethylene (PE), polypropylene (PP), a blend of PE and PP, or a multi-layered structured particle of PE and/or PP.

The material of the particle can comprise polyethylene terephthalate (PET), polyvinylidene fluoride (PVdF), polyamides (Nylons), polyurethanes, polycarbonates, polyesters, polyetheretherketones (PEEK), polyethersulfones (PES), polyimides (PI), polyamide-imides, polyethers, polyoxymethylene (e.g., acetal), polybutylene terephthalate, polyethylenenaphthenate, polybutene, polyolefin copolymers, acrylonitrile-butadiene styrene copolymers (ABS), polystyrene copolymers, polymethylmethacrylate (PMMA), polyvinyl chloride (PVC), polysiloxane polymers (such as polydimethylsiloxane (PDMS)), polybenzimidazole (PBI), polybenzoxazole (PBO), polyphenylenes, polyarylene ether ketones, polyperfluorocyclobutanes, polytetrafluoroethylene (PTFE), polyvinylidene fluoride copolymers and terpolymers, polyvinylidene chloride, polyvinylfluoride, liquid crystalline polymers, polyaramides, polyphenylene oxide, and/or combinations thereof.

Different particle densities are consistent with the present disclosure. The density of a particle can be, be about, be at least, be at least about, be at most, or be at most about, 0.01 g/cm³, 0.02 g/cm³, 0.03 g/cm³, 0.04 g/cm³, 0.05 g/cm³, 0.06 g/cm³, 0.07 g/cm³, 0.08 g/cm³, 0.09 g/cm³, 0.1 g/cm³, 0.2 g/cm³, 0.3 g/cm³, 0.4 g/cm³, 0.5 g/cm³, 0.6 g/cm³, 0.7 g/cm³, 0.8 g/cm³, 0.9 g/cm³, 1 g/cm³, 1.1 g/cm³, 1.2 g/cm³, 1.3 g/cm³, 1.4 g/cm³, 1.5 g/cm³, 1.6 g/cm³, 1.7 g/cm³, 1.8 g/cm³, 1.9 g/cm³, 2 g/cm³, 2.1 g/cm³, 2.2 g/cm³, 2.3 g/cm³, 2.4 g/cm³, 2.5 g/cm³, 2.6 g/cm³, 2.7 g/cm³, 2.8 g/cm³, 2.9 g/cm³, 3 g/cm³, 3.1 g/cm³, 3.2 g/cm³, 3.3 g/cm³, 3.4 g/cm³, 3.5 g/cm³, 3.6 g/cm³, 3.7 g/cm³, 3.8 g/cm³, 3.9 g/cm³, 4 g/cm3, 4.1 g/cm³, 4.2 g/cm³, 4.3 g/cm³, 4.4 g/cm³, 4.5 g/cm³, 4.6 g/cm³, 4.7 g/cm³, 4.8 g/cm³, 4.9 g/cm³, 5 g/cm³, 5.1 g/cm³, 5.2 g/cm³, 5.3 g/cm³, 5.4 g/cm³, 5.5 g/cm³, 5.6 g/cm³, 5.7 g/cm³, 5.8 g/cm³, 5.9 g/cm³, 6 g/cm³, 6.1 g/cm³, 6.2 g/cm³, 6.3 g/cm³, 6.4 g/cm³, 6.5 g/cm³, 6.6 g/cm³, 6.7 g/cm³, 6.8 g/cm³, 6.9 g/cm³, 7 g/cm³, 7.1 g/cm³, 7.2 g/cm³, 7.3 g/cm³, 7.4 g/cm³, 7.5 g/cm³, 7.6 g/cm³, 7.7 g/cm³, 7.8 g/cm³, 7.9 g/cm³, 8 g/cm³, 8.1 g/cm³, 8.2 g/cm³, 8.3 g/cm³, 8.4 g/cm³, 8.5 g/cm³, 8.6 g/cm³, 8.7 g/cm³, 8.8 g/cm³, 8.9 g/cm³, 9 g/cm³, 9.1 g/cm³, 9.2 g/cm³, 9.3 g/cm³, 9.4 g/cm³, 9.5 g/cm³, 9.6 g/cm³, 9.7 g/cm³, 9.8 g/cm³, 9.9 g/cm³, 10 g/cm³, 11 g/cm³, 12 g/cm³, 1³ g/cm³, 14 g/cm³, 15 g/cm³, 16 g/cm³, 17 g/cm³, 18 g/cm³, 19 g/cm³, 20 g/cm³, or a number or a range between any two of these values. For example, the density of a particle can be about 0.1 g/cm³ to about 10 g/cm³.

Particle radius (or diameter) can be selected so as to modulate eventual binding site separation distance (such as the minimum separation distance between two binding sites) on the planar surface, assembly of particles into regular or irregular regions on the planar surface, or for other rationales. Particles are often selected such that a radius (or diameter) of one, one or more, or each, of the plurality of particles is about 10⁻⁹ m to about 10⁻⁴ m. A volume of one, one or more, or each, of the plurality of particles can be about 10⁻² m³ to about 10⁻¹² m³. Some particle populations exhibit some or total uniformity of size, such that two of the plurality of particles have an identical or about identical radius, and/or each of the plurality of particles has an identical radius.

Alternately, some particle populations are heterogeneous, for example so as to modulate the probability or proportion of the particle distribution that is irregular or random. A population of particles can include 1%, 2%, 3%, 4%, 5%, 6%, 7%, 8%, 9%, 10%, 20%, 30%, 40%, 50%, 60%, 70%, 80%, 90%, 99%, or more, of the plurality of particles. A population of particles (or a subset of particles of the plurality of particles) can have an identical radius (or diameter). For example, a first population of particles can have a first identical radius (or diameter). A second population of particles having a second identical radius (or diameter). The first identical radius and the second identical radius can be different. The two populations of particles within the plurality of particles is referred to herein as polydispersity. The first identical radius (or diameter) and the second identical radius (or diameter) can differ by, by about, by at least, by at least about, by at most, or by at most about, for example, 0.01 μm, 0.02 μm, 0.03 μm, 0.04 μm, 0.05 μm, 0.06 μm, 0.07 μm, 0.08 μm, 0.09 μm, 0.1 μm, 0.11 μm, 0.12 μm, 0.13 μm, 0.14 μm, 0.15 μm, 0.16 μm, 0.17 μm, 0.18 μm, 0.19 μm, 0.2 μm, 0.3 μm, 0.4 μm, 0.5 μm, 0.6 μm, 0.7 μm, 0.8 μm, 0.9 μm, 1 μm, or a number or a range between any two of these values. The first identical radius and the second identical radius can differ by, by about, by at least, by at least about, by at most, or by at most about, for example, 1%, 2%, 3%, 4% 5%, 6%, 7%, 8%, 9%, 10%, 11%, 12%, 13%, 14%, 15%, 16%, 17%, 18%, 19%, 20%, of the first identical radius (or the second identical radius). The first identical radius (or diameter) can be bigger than the second identical radius (or diameter). For example, the first identical radius is 0.5 μm, and the second identical radius can be 0.4 μm. The first identical radius (or diameter) can be smaller than the second identical radius (or diameter). A ratio of a number of the first pollution of particles and a number of the second population of particles can be about 1:100 to about 100:1. The ratio can be, be about, be at least, be at least about, be at most, or be at most about 1:100, 1:99, 1:98, 1:97, 1:96, 1:95, 1:94, 1:93, 1:92, 1:91, 1:90, 1:89, 1:88, 1:87, 1:86, 1:85, 1:84, 1:83, 1:82, 1:81, 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of these values. The variation in bead size can also be descried as the standard deviation of bead diameter divided by the average bead diameter. Such variation may be at least 0.02, at least 0.05, at least 0.1, or at least 0.2, such as between 0.01 and 0.2, or between 0.02 and 0.1. An irregular array within a field of view usually comprise a plurality of disjunctions and/or a plurality of crystal latices separated by a disjunction. Such field of view may be at least 0.0001 mm², 0.0002 mm², 0.0005 mm², 0.001 mm², 0.002 mm², 0.005 mm², 0.01 mm², 0.02 mm², 0.05 mm², 0.1 mm², 0.2 mm², 0.5 mm², or 1 mm². In certain aspects, a grain size of a structured surface may be less than 300, less than 250, less than 200, less than 150, less than 100, less than 50, or less than 20.

Particles are often spherical, such that one, one or more, or each, of the plurality of particles has a spherical shape, although other shapes are also consistent with the disclosure herein. The plurality of particles deposited on a particular planar surface may comprises about 10⁴ particles to about 10⁸ particles, although numbers greater or less than these values may be employed, such as when smaller or larger planar surfaces are employed or smaller or larger particle sizes are used, for example to modulate binding site minimum distance or pitch.

The radius (or diameter) of one, one or more, or each particle (or the distance between the centers of two particles) can vary. The radius (or diameter) of one, one or more, or each particle (or the distance between the centers of two particles) can be, be about, be at least, be at least about, be at most, or be at most about, 10 nanometer (nm), 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm, 1000 nm, 2 micrometer (μm), 3 μm, 4 μm, 5 μm, 6 μm, 7 μm, 8 μm, 9 μm, μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, or a number or a range between any two of these values. For example, the radius (or diameter) of one, one or more, or each, of the plurality of particles is about 1 nm to about 100 μm. Two (or more, such as 1%, 2%, 3%, 4%, 5%, 6%, 7% 8%, 9% 10%, 20%, 30%, 40%, 50%, 70%, 80%, 90%, 99%, or more, of the plurality of particles) particles have an identical radius. Each of the plurality of particles can have an identical radius. One, one or more, or each, of the plurality of particles has a spherical shape. Other particle shapes are also contemplated by the disclosure.

The volume of one, one or more, or each, of the plurality of particles can be different (or the same). The volume of one, one or more, or each, of the plurality of particles can be, be about, be at least, be at least about, be at most, or be at most about, 1,000 nm³, 10,000 nm³, 10,0000 μm³, 1,000,000 nm³, 10,000,000 nm³, 100,000,000 μm³, 1,000,000,000 nm³, 2 μm³, 3 μm³, 4 μm³, 5 μm³, 6 μm³, 7 μm³, 8 μm³, 9 μm³, 10 μm³, 20 μm³, 30 μm³, 40 μm³, 50 μm³, 60 μm³, 70 μm³, 80 μm³, 90 μm³, 100 μm³, 200 μm³, 300 μm³, 400 μm³, 500 μm³, 600 μm³, 700 μm³, 800 μm³, 900 μm³, 1,000 μm³, 10,000 μm³, 100,000 μm³, 1,000,000 μm³, or a number or a range between any two of these values. The volume of one, one or more, or each, of the plurality of particles can be, be about, be at least, be at least about, be at most, or be at most about, 1 nanolieter (nl), 2 nl, 3 nl, 4 nl, 5 nl, 6 nl, 7 nl, 8 nl, 9 nl, 10 nl, 11 nl, 12 nl, 13 nl, 14 nl, 15 nl, 16 nl, 17 nl, 18 nl, 19 nl, 20 nl, 21 nl, 22 nl, 23 nl, 24 nl, 25 nl, 26 nl, 27 nl, 28 nl, 29 nl, 30 nl, 31 nl, 32 nl, 33 nl, 34 nl, 35 nl, 36 nl, 37 nl, 38 nl, 39 nl, 40 nl, 41 nl, 42 nl, 43 nl, 44 nl, 45 nl, 46 nl, 47 nl, 48 nl, 49 nl, 50 nl, 51 nl, 52 nl, 53 nl, 54 nl, 55 nl, 56 nl, 57 nl, 58 nl, 59 nl, 60 nl, 61 nl, 62 nl, 63 nl, 64 nl, 65 nl, 66 nl, 67 nl, 68 nl, 69 nl, 70 nl, 71 nl, 72 nl, 73 nl, 74 nl, 75 nl, 76 nl, 77 nl, 78 nl, 79 nl, 80 nl, 81 nl, 82 nl, 83 nl, 84 nl, 85 nl, 86 nl, 87 nl, 88 nl, 89 nl, 90 nl, 91 nl, 92 nl, 93 nl, 94 nl, 95 nl, 96 nl, 97 nl, 98 nl, 99 nl, 100 nl, or a number or a range between any two of these values. For example, the volume of one, one or more, or each, of the plurality of particles is about 1 nm³ to about 1,000,000 μm³.

Different numbers of particles are contemplated herein. The number of particles in the plurality of particles (or the number of particles in a subset of particles) can be, be about, be at least, be at least about, be at most, or be at most about, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, 10,000,000, 20,000,000, 30,000,000, 40,000,000, 50,000,000, 60,000,000, 70,000,000, 80,000,000, 90,000,000, 100,000,000, 200,000,000, 300,000,000, 400,000,000, 500,000,000, 600,000,000, 700,000,000, 800,000,000, 900,000,000, 1,000,000,000, or a number or a range between any two of these values. For example, the number of particles in the plurality of particles is about 10,000, particles to about 10,000,000 particles.

The plurality of particles (or a subset of particles) can be present at different densities depending on, for example, the size of the surface area of the liquid where the plurality of particles is delivered to, the number of particles delivered to the liquid, and the size (e.g., the radius or the volume) of the particles delivered to the liquid. The plurality of particles (or a subset of particles) can be present at a density (e.g., a local density or an average density) of, of about, of at least, of at least about, of at most, or of at most about, 100 k/mm², 110 k/mm², 120 k/mm², 130 k/mm², 140 k/mm², 150 k/mm², 160 k/mm², 170 k/mm², 180 k/mm², 190 k/mm², 200 k/mm², 300 k/mm², 400 k/mm², 500 k/mm², 600 k/mm², 700 k/mm², 800 k/mm², 900 k/mm², 1000 k/mm², 1100 k/mm², 1200 k/mm², 1300 k/mm², 1400 k/mm², 1,500 k/mm², 1600 k/mm², 1700 k/mm², 1800 k/mm², 1900 k/mm², 2000 k/mm², 2100 k/mm², 2200 k/mm², 2300 k/mm², 2400 k/mm², 2500 k/mm², 2600 k/mm², 2700 k/mm², 2800 k/mm², 2900 k/mm², 3000 k/mm², 3100 k/mm², 3200 k/mm², 3300 k/mm², 3400 k/mm², 3500 k/mm², 3600 k/mm², 3700 k/mm², 3800 k/mm², 3900 k/mm², 4000 k/mm², 4100 k/mm², 4²00 k/mm², 4300 k/mm², 4400 k/mm², 4500 k/mm², 4600 k/mm², 4700 k/mm², 4800 k/mm², 4900 k/mm², 5000 k/mm², 6000 k/mm², 7000 k/mm², 8000 k/mm², 9000 k/mm², 10000 k/mm², or a number or a range between any two of these values. For example, the plurality of particles is present at a density of about 200 k/mm² to about 2,000 k/mm². For example, the plurality of particles is present at a density of at least 600 k/mm², at least 800 k/mm², or at least 1,000 k/mm².

### Particle Configuration

Depositing the plurality of particles to the surface of the liquid can include packing (e.g., by colloidal self-assembly) the plurality of beads into a configuration comprising a first crystal lattice and a second crystal lattice separated by a disjunction on the surface of the liquid (and the surface of the planar structure after the liquid between the surface of the liquid and the surface of the planar structure is removed). The plurality of particles can be packed so as to form a region having regularly positioned/ordered beads and a region having randomly irregularly positioned/non-ordered beads on the surface of the liquid. The plurality of particles can be randomly packed on the surface of the liquid, for example, when an insufficient number of particles are introduced to the surface of the liquid. A disjunction can also be referred to as a local disjunction.

Particles can be present with different particle configurations comprising crystal lattices of ordered particles separated by disjunctions. Any crystal lattice of the present disclosure can have (or can be described using or as having) a configuration, such as the hexagonal configuration, the equilateral triangle configuration, straight line configuration, and the linear configurations. Different configurations of the crystal lattice (such as the arrangement or the relative positions or locations of the particles and the resultant binding sites (or active sites) after etching) may be present in different embodiments. The size and the location of each crystal lattice is not predetermined while the particles in the crystal lattice are ordered and tightly packed. Two crystal lattices can be separated by a disjunction. Referring to FIG. 3A a crystal lattice can be described as having a hexagonal configuration 308h1, 308h2. For example, the first crystal lattice 302r1 comprises the first subset of first particles 304r1 in a first hexagonal configuration 308h1. The first crystal lattice 302r1 can comprise the first subsets each with seven first particles 304r1 in an identical first hexagonal configuration (in terms of rotation) 308h1. Seven first particles of the first subset of first particles in the first hexagonal configuration 308h1 can be at the six vertices and the center of a first hexagon 310h1. Each of the six first particles at the six vertices of the first hexagon 310h1 can be in contact with the first particle at the center of the first hexagon 310hl and two other first particles at the vertices of the first hexagon 310h1. The second crystal lattice 302h2 can comprise the second subset of second particles 304r2 in a second hexagonal configuration 308h2. Seven second particles of the second subset of second particles in the second hexagonal configuration 308h2 can be at the six vertices and the center of a second hexagon 310h2. Each of the six second particles at the six vertices of the second hexagon 310h2 can be in contact with the second particle at the center of the second hexagon 310h2 and two other second particles at the vertices of the second hexagon 310h2. The first hexagonal configuration and the second hexagonal configuration can have an identical orientation. The first hexagon 310h1 and the second hexagon 310h2 can have different orientations. As illustrated in FIG. 3A, the first hexagon 310h1 and the second hexagon 310h2 are related to each other by a rotation (and a translation). The hexagons in a hexagonal configuration can share sides. The hexagons in different hexagonal configurations may not share sides. The hexagons in a hexagonal configuration can be congruent (e.g., having the same size and shape). The hexagons in different hexagonal configurations can be congruent (e.g., having the same size and shape) or different. The hexagons in different hexagonal configuration can be related by a rotation (and a translation). The hexagons in different hexagonal configurations can be related by a translation (not a rotation) such that the hexagons have the same orientation. The particles having the hexagonal configuration can be tightly packed and ordered in a non-predetermined manner generated with colloidal self-assembly. The two crystal lattices 302r1, 302r2 illustrated in FIG. 3A are separated by a disjunction 306d containing no particle.

Referring to FIG. 3B, a crystal lattice can have (or be described as having) an equilateral triangle configuration 308t1, 308t2. For example, the first crystal lattice 302r1 comprises the first subset of first particles 304r1 in a first equilateral triangle configuration 308t1 such that three adjacent non-colinear first particles of the first subset of first particles form a first equilateral triangle 310t1. The second crystal lattice 308t2 can comprise the second subset of second particles 304r2 in a second equilateral triangle configuration 308t2 such that any three adjacent non-colinear second particles of the second subset of second particles form a second equilateral triangle 310t2. The first equilateral triangle configuration 308t1 and the second equilateral triangle configuration 308t2 can be different. The first equilateral triangle 310t1 and the second equilateral triangle 310t2 can have different orientations. For example, the first equilateral triangle 310t1 and the second equilateral triangle 310t2 are related to each other by a rotation (and a translation). The equilateral triangles in an equilateral triangle configuration can share sides. The equilateral triangles in different equilateral triangle configurations may not share sides. The equilateral triangles in an equilateral triangle configuration can be congruent (e.g., having the same size and shape). An equilateral triangle configuration can include two subsets of equilateral triangles. Equilateral triangles in each subset can be related by translation (not rotation). Equilateral triangles between the two subsets are related by a rotation of 180° (and a translation). The equilateral triangles in different equilateral triangles configurations can be congruent (e.g., having the same size and shape) or different. The equilateral triangle in different equilateral triangles configurations can be related by a rotation (and a translation) as illustrated in FIG. 3B. The equilateral triangles in different equilateral triangles configurations can be related by a translation (not a rotation), such that the equilateral triangles have the same orientation for corresponding subsets of equilateral triangles. The particles having an equilateral triangle configuration can be tightly packed and ordered in a non-predetermined manner generated with colloidal self-assembly. The two crystal lattices 302r1, 302r2 illustrated in FIG. 3B are separated by a disjunction 306d containing no particle.

Referring to FIG. 3C, a crystal lattice can have (or be described as having) a straight line configuration 308s1, 308s2. A first straight line 310s1 drawn between adjacent first particles 304r1 in the first crystal lattice 302r1 and any second straight line 310s1 drawn between adjacent second particles 304r2 in the second crystal lattice 302r2 are not parallel. For example, the first straight line 310s1 and the second straight line 310s2 are not parallel such that the two lines are related to each other by a rotation other than an integer multiple of 60° (and a translation) as illustrated in FIG. 3C. As an example, the first straight line 310s1 and the second straight line 310s2 are not parallel such that the two lines are related to each other by a rotation of an integer multiple of 60° (and a translation). As another example, the first straight line 310s1 and the second straight line 310s2 can have an identical direction/are parallel such that the two straight lines are related by a translation (not a rotation). The straight lines in different straight line configurations can have an identical direction/can be parallel such that the straight lines are related by a rotation (and a translation). The straight lines in different straight line configurations can be related by a translation (not a rotation), such that the straight lines have the same direction. The particles having the straight line configuration can be tightly packed and ordered in a non-predetermined manner generated with colloidal self-assembly. In FIG. 3C, the disjunction 306d that separates the two crystal lattices 302r1, 302r2 includes non-ordered (or randomly distributed or disordered) particles 306i.

Referring to FIG. 3D, a crystal lattice can have (or be described as having) a linear configuration 308l1, 308l2. The first crystal lattice 302r1 comprises the first subset of first particles 304r1 arranged in a first plurality of first rows 310l1 of first particles. First particles in each first row 310l1 of the first plurality of first rows can be arranged in a first linear configuration 308l1 such that a first particle in the first row is in contact with two first particles adjacent to the first particle in the first row. Each first particle in the first row 310l1 (other than the first particles at the end of a row) can be in contact with two first particles adjacent to the first particle in the first row. Two adjacent first rows 310l1 can be offset by a first offset 312o1 of the first linear configuration. in a first direction 314d1 of the first linear configuration 308l1. The first offset 312o1 can be the same (or substantially the same as) the radius of a first particle of the plurality of first particles. The two adjacent rows 310l1 can be offset by a second offset 316o1 of the first linear configuration, such as a diameter of the particle of the plurality of particles, in a second direction 318d1 of the first linear configuration. The second offset 316o1 can be greater than a radius of a first particle of the plurality of first particles and less than a diameter of the first particle of the plurality of particles, such as the square root of three multiplied by the radius of the first particle. The second direction 318d1 of the first linear configuration can be perpendicular to the first direction 314d1 of the first linear configuration. A first particle in one first row can be in contact with two adjacent first particles in an adjacent first row.

With continued reference to FIG. 3D, the second crystal lattice 302r1 can comprise a second subset of second particles 304r2 arranged in a second plurality of second rows 310l2 of second particles. Second particles in each second row of the second plurality of second rows can be arranged in a second linear configuration 308l2 such that a second particle in the second row is in contact with two second particles adjacent to the second particle in the second row. Each second particle in the second row 310l2 (other than the second particles at the end of a row) can be in contact with two second particles adjacent to the second particle in the second row. Two adjacent second rows 310l2 can be offset by a first offset 312o2 of the second linear configuration in a first direction 314d2 of the second linear configuration. The first offset 312o2 can be greater than a radius of a second particle of the plurality of second particles and less than a diameter of the second particle of the plurality of particles, such as the square root of three multiplied by the radius of the second particle. The two adjacent rows 310l2 can be offset by a second offset 314d2 of the second linear configuration, such as a diameter of the second particle of the plurality of second particles, in a second direction 318d2 of the second linear configuration. The second direction 318d2 of the second linear configuration can be perpendicular to the first direction 314d1 of the second linear configuration. A second particle in one second row can be in contact with two adjacent second particles in an adjacent second row. In FIG. 3D, the disjunction 306d that separates the two crystal lattices 302r1, 302r2 includes non-ordered (or randomly distributed or disordered) particles 306i.

One or more parameters described with reference to the first linear configuration 308l1 and the second linear configuration 308l2 can be the same or different. The first linear configuration 308l1 and the second linear configuration 308l2 can be different (or the same), for example, with respect to some or all of the parameters described with reference to the linear configurations. The orientations of the linear configurations can be different (or the same). The first direction 314d1 of the first linear configuration and the first direction 314d2 of the second linear configuration can be different (or the same). The second direction 318d1 of the first linear configuration and the second direction 318d2 of the second linear configuration can be different (or the same). The combination of the first direction 314d1 and the second direction 318d1 of the first linear configuration and the combination of the first direction 314d2 and the second direction 314d2 of the second linear configuration can be different (or the same). For example, the first angle formed by the intersection of the first direction and the second direction of the first linear configuration and the second angle formed by the intersection of the first direction and the second direction of the second linear configuration are related to each other by a rotation (and a translation). FIG. 3D illustrates the first angle formed by the intersection of the first direction and the second direction of the first linear configuration and the second angle formed by the intersection of the first direction and the second direction of the second linear configuration are related to each other not a translation.

The offsets of the linear configurations can be different (or the same). The first offset 312o1 of the first linear configuration and the first offset 312o2 of the second linear configuration can be identical, or within a percentage of each other (or different). The second offset 316o1 of the first linear configuration and the second offset 316o2 of the second linear configuration can be identical, or within a percentage or more of each other. The percentage difference between the corresponding offsets can be different in different embodiments. The percentage can be, be about, be at least, be at most, or be at most about, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values.

In some embodiments, one or more irregular arrays form at the surface of the liquid. An irregular array can result from, for example, a mixture of particles with two different sizes present on the surface of the liquid and are then deposited onto the planar structure. Two irregular arrays can be separated by a disjunction. Each particle in an irregular array (e.g., a first irregular array or a second irregular array) can be at or greater than a threshold distance away from a nearest neighbor particle of the particle in the irregular array. The threshold distance can be a radius of the particle. The threshold distance can be, be about, be at least, be at least about, be at most, or be at most about, 0.1 μm, 0.2 μm, 0.3 μm, 0.4 μm, 0.5 μm, 0.6 μm, 0.7 μm, 0.8 μm, 0.9 μm, 1 μm, 1.1 μm, 1.2 μm, 1.3 μm, 1.4 μm, 1.5 μm, 1.6 μm, 1.7 μm, 1.8 μm, 1.9 μm, 2 μm, 3 μm, 4 μm, 5 μm, 6 μm, 7 μm, 8 μm, 9 μm, 10 μm, or a number or a range between any two of these values. An irregular array can comprise no seven neighbor particles that are at six vertices and a center of any hexagon. An irregular array can comprise no six neighbor particles surrounding a 7th particle at six vertices of a hexagon. An irregular array can comprise seven neighbor particles, six of which are at six vertices of a six-sided shape that is not a hexagon and surround a 7th particle of the seven neighbor particles. An irregular array can comprise no particles in a hexagonal configuration, an equilateral triangle configuration, a straight line configuration, or a linear configuration. Particles not being in a hexagonal configuration means that a hexagonal grid (e.g., a hexagonal grid shown in FIG. 3A) cannot be aligned with the particles. Particles not being in an equilateral triangle configuration means that a triangle grid (e.g., a triangle grid shown in FIG. 3B) cannot be aligned with the particles. Particles not being in a linear configuration means that a linear grid (e.g., a linear grid shown in FIG. 3C) cannot be aligned with the particles. Particles not being in a straight line configuration means that a straight line grid (e.g., a straight line grid shown in FIG. 3D) cannot be aligned with the particles. An irregular array can comprise no particles within at least a threshold distance of each other that are in a hexagonal configuration, an equilateral triangle configuration, a straight line configuration, or a linear configuration. The threshold distance can be, be about, be at least, be at least about, be at most, or be at most about, 0.1 μm, 0.2 μm, 0.3 μm, 0.4 μm, 0.5 μm, 0.6 μm, 0.7 μm, 0.8 μm, 0.9 μm, 1 μm, 1.1 μm, 1.2 μm, 1.3 μm, 1.4 μm, 1.5 μm, 1.6 μm, 1.7 μm, 1.8 μm, 1.9 μm, 2 μm, 3 μm, 4 μm, 5 μm, 6 μm, 7 μm, 8 μm, 9 μm, 10 μm, or a number or a range between any two of these values.

### Planar Structure

The planar structure can comprise a surface durable enough to survive the etching process. The planar structure can be a substrate or a flow cell surface. The material of the planar structure (or substrate or flow cell surface) can comprise silicon, silicon nitride glass, borosilicate glass, quartz, fused quartz, silica, fused silica, a metal, a ceramic, plastic, or a combination thereof. In some embodiments, a material of the masking layer comprises aluminum, indium tin oxide, chromium, copper, gallium arsenide, gold, molybdenum, platinum, silicon, silicon dioxide, silicon nitride, silver, tantalum, titanium, titanium nitride, tungsten, or a combination thereof.

The material of the planar structure (or substrate or flow cell surface) can comprise in whole or in part one or more polymeric materials, such as polyethylene or polyethylene derivatives, such as cyclic olefin copolymers (COC), polymethylmethacrylate (PMMA), polydimethylsiloxane (PDMS), polycarbonate, polystyrene, polypropylene, polyvinyl chloride, polytetrafluoroethylene, polyoxymethylene, polyether ether ketone, polycarbonate, or polystyrene. The material of the planar structure can comprise in whole or in part inorganic materials, such as silicon, or other silica based materials, e.g., glass, quartz, fused silica, borosilicate glass, metals, and ceramics.

### Removing the Liquid

The liquid between the particles and the planar structure can be removed through various approaches. Removing the liquid between the particles and the planar structure comprises removing the liquid from a chamber (or container) containing the liquid, the planar surface, and the particles. Removing the liquid between the particles and the planar structure comprises draining the liquid from a chamber (or container) containing the liquid, the planar surface, and the particles. Removing the liquid between the particles and the planar structure comprises heating the liquid. Removing the liquid between the particles and the planar structure comprises allowing the liquid between the particles and the planar structure to evaporate. The liquid with the desired properties, such as surface tension and evaporation rate, can be used. Removing the liquid between the particles and the planar structure comprises evaporating the liquid between the particles and the planar structure to evaporate. Removing the liquid between the particles and the planar structure comprises elevating the planar structure above the surface of the liquid. The liquid can be removed without disrupting (or substantially disrupting) the particle configurations and the crystal lattices of the particle configurations. With the liquid removed, the packing of the particles on the surface of the liquid is transferred to the surface of the planar structure undisrupted (or substantially undisrupted)

### Etching

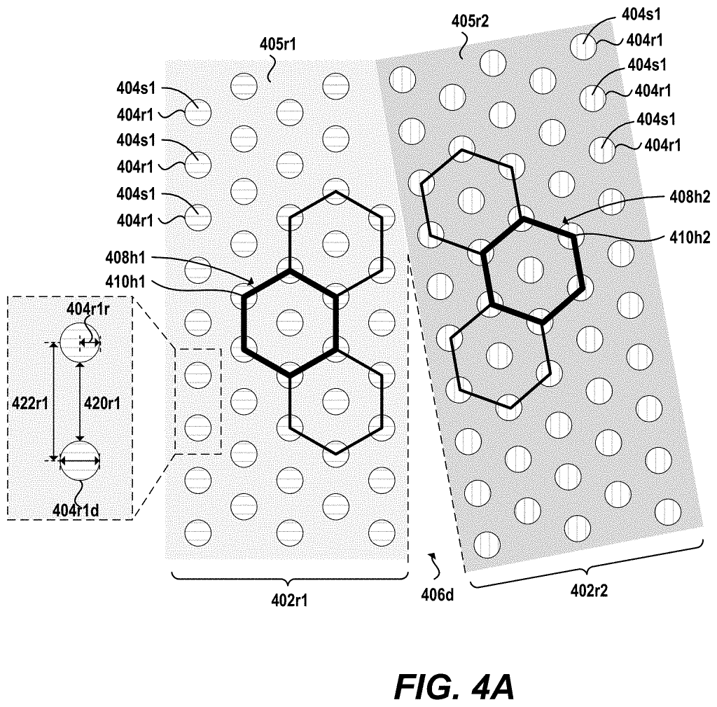

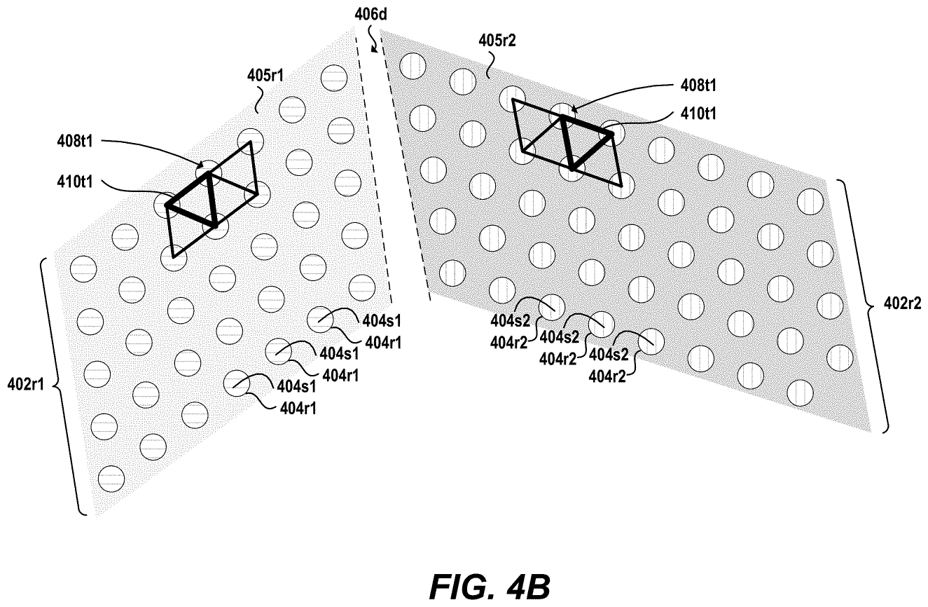

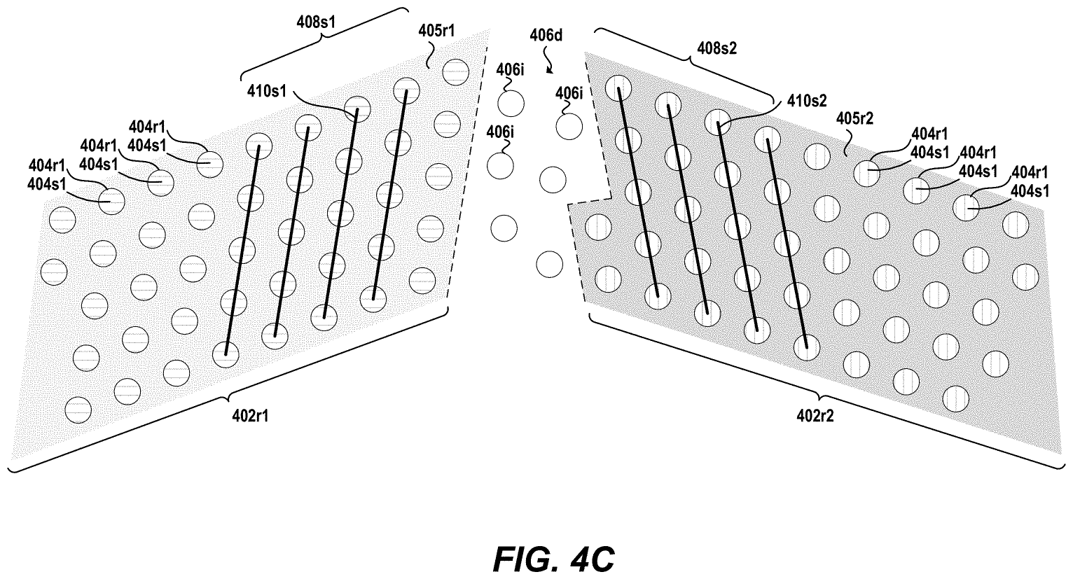

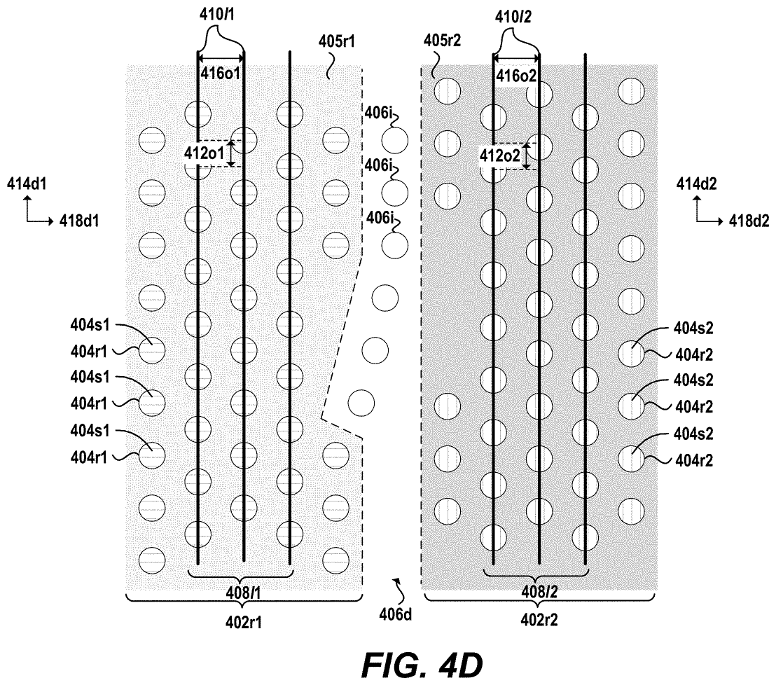

The method can comprise an etching process. A number of etching approaches are consistent with the disclosure herein. The planar structure and the plurality of particles in contact with the planar structure are subjected to etching, for example so as to recapitulate or mirror the particle distribution or configuration on the etched planar surface as binding sites. Referring to FIGS. 4A-4D, the etching process can include etching the planar structure and the plurality of particles in contact with the planar structure to generate a plurality of retained regions 404r1, 404r2 where a binding site layer (or a substance layer, a reaction site layer, or an active site layer) on the planar surface is in contact (for example, directly, or indirectly via a masking layer) with, is shielded by, or is in close proximity to the plurality of particles prior to or during etching and is differentially retained after etching. For example, the plurality of retained regions 404r1, 404r2 of the substance layer on the planar surface is in contact with the plurality of retained regions 404r1, 404r2 of a masking layer that are in contact with the plurality of particles and is differentially retained. The plurality of retained regions 404r1, 404r2 can specify, or can comprise, a plurality of binding sites (or active sites or reaction sites) 404s1, 404s2. The plurality of retained regions 404r1, 404r2 can specify, or can comprise, a plurality of active sites 404s1, 404s2. Etching the planar structure and the plurality of particles in contact with the planar structure can generate a plurality of etched regions 405r1, 405r2 where the substance layer is not in contact (for example, directly or indirectly via a masking layer) with, not shielded by, or not in close proximity to the plurality of particles and is differentially removed. For example, the plurality of etched regions 405r1, 405r2 of the substance layer on the planar surface is not in contact with the plurality of etched regions 405r1, 405r2 of the masking layer, which in turn are not in contact with the plurality of particles. The plurality of etched regions 405r1, 405r2 is differentially retained. FIGS. 4A-4D illustrate circular (or substantially circular) retained regions.

The size of a retained region 404r1 can be, for example, a radius 404r1r or a diameter 404r1d of the retained region for the circular regions illustrated in FIG. 4A. The size of a retained region 404r1 of the plurality of retained regions can be smaller, by a percentage, than the size of the particle of the plurality of particles that is in contact with the retained region before etching or after the etching process, such as the radius 304r1r or the diameter 304r1d of particles illustrated in FIG. 3A. Referring to FIG. 4A, the percentage that the retained region 404r1 is smaller than the particle can be, be about, be at least, be at least about, be at most, or be at most about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. The size of a retained region 404r1 of the plurality of retained regions can be larger, by a percentage, than the size of the particle of the plurality of particles that is in contact with the retained region after the etching process. The percentage that the retained region 404r1 is larger than the particle after the etching can be, be about, be at least, be at least about, be at most, or be at most about, 1%, 2%, 3%, 4%, 5%, 6%, 7%, 8%, 9%, 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, or a number or a range between any two of these values.

The etching process can comprise degrading a portion of one, one or more, or each, particle of the plurality of particles. The degrading can determine the size of one, one or more, or each, binding site of the plurality of binding sites on the planar structure corresponding to a retained region of the plurality of retained regions the at least one particle is in contact with the planar surface. The degrading can determine or affect a separation distance 420r1 between two adjacent binding sites of the plurality of binding sites on the planar structure. The separation distance 420r1 can be measured from the closest edges of the two adjacent binding sites. The pitch 422r1 between two adjacent binding sites of the plurality of binding sites illustrated in FIG. 4A is the same as (or substantially the same as) the pitch of the particles 322r1 illustrated in FIG. 3A, which is in turn determined or affected by the size of the particles. The etching process can include removing any of the plurality of particles, or any portion of each bead remaining, subsequent to the etching.

Numerous etching methods and etching agents are consistent with the disclosure herein. The etching process can comprise plasma etching, reactive ion etching (RIE), capacitive RIE, inductive RIE, deep reactive ion etching, chemical vapor deposition (CVD), plasma-enhanced CVD (PECVD), or a combination thereof. The etching process can comprise isotropic etching, directional etching, vertical etching, or a combination thereof. The etching process can comprise etching using one gas, or two or more gasses, selected from a group consisting of O₂, CF₄, C₂F₆, C₄F₈, CHF₃, SF₆, NF₃, BCl₃, Cl₂, HBr, and Ar. The gases can be etching agents. The etching process can comprise etching using oxygen gas, carbon tetrafluoride gas, or a combination thereof. The ratio of any two gases of the two or more gases can be different in different embodiments. The ratio of any two gases of the two or more gases can be, be about, be at least, be at least about, be at most, or be at most about, 1:100, 1:99, 1:98, 1:97, 1:96, 1:95, 1:94, 1:93, 1:92, 1:91, 1:90, 1:89, 1:88, 1:87, 1:86, 1:85, 1:84, 1:83, 1:82, 1:81, 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any two of these values. For example, the ratio of any two gases of the two or more gases can be about 1:100 to about 100:1.

The mass flow rate of any gas used in the etching process, or the total mass flow rate of the two or more gases used in the etching process, can be adjusted. The mass flow rate of any gas used in the etching process, or the total mass flow rate of the two or more gases used in the etching process, can be, be about, be at least, be at least about, be at most, or be at most about, 0.1 standard cubic centimeter per minute (sccm), 0.2 sccm, 0.3 sccm, 0.4 sccm, 0.5 sccm, 0.6 sccm, 0.7 sccm, 0.8 sccm, 0.9 sccm, 1 sccm, 2 sccm, 3 sccm, 4 sccm, 5 sccm, 6 sccm, 7 sccm, 8 sccm, 9 sccm, 10 sccm, 11 sccm, 12 sccm, 13 sccm, 14 sccm, 15 sccm, 16 sccm, 17 sccm, 18 sccm, 19 sccm, 20 sccm, 21 sccm, 22 sccm, 23 sccm, 24 sccm, 25 sccm, 26 sccm, 27 sccm, 28 sccm, 29 sccm, 30 sccm, 31 sccm, 32 sccm, 33 sccm, 34 sccm, 35 sccm, 36 sccm, 37 sccm, 38 sccm, 39 sccm, 40 sccm, 41 sccm, 42 sccm, 43 sccm, 44 sccm, 45 sccm, 46 sccm, 47 sccm, 48 sccm, 49 sccm, 50 sccm, 51 sccm, 52 sccm, 53 sccm, 54 sccm, 55 sccm, 56 sccm, 57 sccm, 58 sccm, 59 sccm, 60 sccm, 61 sccm, 62 sccm, 63 sccm, 64 sccm, 65 sccm, 66 sccm, 67 sccm, 68 sccm, 69 sccm, 70 sccm, 71 sccm, 72 sccm, 73 sccm, 74 sccm, 75 sccm, 76 sccm, 77 sccm, 78 sccm, 79 sccm, 80 sccm, 81 sccm, 82 sccm, 83 sccm, 84 sccm, 85 sccm, 86 sccm, 87 sccm, 88 sccm, 89 sccm, 90 sccm, 91 sccm, 92 sccm, 93 sccm, 94 sccm, 95 sccm, 96 sccm, 97 sccm, 98 sccm, 99 sccm, 100 sccm, 120 sccm, 130 sccm, 140 sccm, 150 sccm, 160 sccm, 170 sccm, 180 sccm, 190 sccm, 200 sccm, or a number or a range between any two of these values. For example, the mass flow rate of any gas used in the etching process, or the total mass flow rate of the two or more gases is about 1 sccm to about 100 sccm.

The etching process can include one or more steps. For example, the etching process comprises performing two or more etching steps. The etching process can comprises performing two or more etching steps using different gases. For example, the first etching step can use oxygen gas, and the second etching step can use oxygen gas and carbon tetrafluoride gas. The etching process can comprise performing two or more etching steps using a first gas in the first etching step and a second gas in the second etching step. The first gas and the second gas can be different. The second etching step can be performed using both the first gas and the second gas. For example, the first etching step can use oxygen gas, and the second etching step can use oxygen gas and carbon tetrafluoride gas.

The etching process (or any step of the etching process) can be performed using different wattages. The etching process (or any step of the etching process) can be performed using a power of, of about, of at least, of at least about, of at most, or of at most about, 1 watt (W), 2 W, 3 W, 4 W, 5 W, 6 W, 7 W, 8 W, 9 W, 10 W, 11 W, 12 W, 13 W, 14 W, 15 W, 16 W, 17 W, 18 W, 19 W, 20 W, 21 W, 22 W, 23 W, 24 W, 25 W, 26 W, 27 W, 28 W, 29 W, 30 W, 31 W, 32 W, 33 W, 34 W, 35 W, 36 W, 37 W, 38 W, 39 W, 40 W, 41 W, 42 W, 43 W, 44 W, 45 W, 46 W, 47 W, 48 W, 49 W, 50 W, 51 W, 52 W, 53 W, 54 W, 55 W, 56 W, 57 W, 58 W, 59 W, 60 W, 61 W, 62 W, 63 W, 64 W, 65 W, 66 W, 67 W, 68 W, 69 W, 70 W, 71 W, 72 W, 73 W, 74 W, 75 W, 76 W, 77 W, 78 W, 79 W, 80 W, 81 W, 82 W, 83 W, 84 W, 85 W, 86 W, 87 W, 88 W, 89 W, 90 W, 91 W, 92 W, 93 W, 94 W, 95 W, 96 W, 97 W, 98 W, 99 W, 100 W, 110 W, 120 W, 130 W, 140 W, 150 W, 160 W, 170 W, 180 W, 190 W, 200 W, or a number or a range between any two of these values. For example, the etching process is performed at a power of about 10 W to about 100 W.

The etching process (or any step of the etching process) can be performed at different pressures. The etching process (or any step of the etching process) can be performed at a pressure of, of about, of at least, of at least about, of at most, or of at most about, 1 millitorr (mT), 2 mT, 3 mT, 4 mT, 5 mT, 6 mT, 7 mT, 8 mT, 9 mT, 10 mT, 11 mT, 12 mT, 13 mT, 14 mT, 15 mT, 16 mT, 17 mT, 18 mT, 19 mT, 20 mT, 21 mT, 22 mT, 23 mT, 24 mT, 25 mT, 26 mT, 27 mT, 28 mT, 29 mT, 30 mT, 31 mT, 32 mT, 33 mT, 34 mT, 35 mT, 36 mT, 37 mT, 38 mT, 39 mT, 40 mT, 41 mT, 42 mT, 43 mT, 44 mT, 45 mT, 46 mT, 47 mT, 48 mT, 49 mT, 50 mT, 51 mT, 52 mT, 53 mT, 54 mT, 55 mT, 56 mT, 57 mT, 58 mT, 59 mT, 60 mT, 61 mT, 62 mT, 63 mT, 64 mT, 65 mT, 66 mT, 67 mT, 68 mT, 69 mT, 70 mT, 71 mT, 72 mT, 73 mT, 74 mT, 75 mT, 76 mT, 77 mT, 78 mT, 79 mT, 80 mT, 81 mT, 82 mT, 83 mT, 84 mT, 85 mT, 86 mT, 87 mT, 88 mT, 89 mT, 90 mT, 91 mT, 92 mT, 93 mT, 94 mT, 95 mT, 96 mT, 97 mT, 98 mT, 99 mT, 100 mT, 200 mT, 300 mT, 400 mT, 500 mT, 600 mT, 700 mT, 800 mT, 900 mT, 1000 mT, 2000 mT, 3000 mT, 4000 mT, 5000 mT, 6000 mT, 7000 mT, 8000 mT, 9000 mT, 10000 mT, or a number or a range between any two of these values. For example, the etching process is performed at a pressure of about 1 mT to about 5000 mT.

The etching process (or any step of the etching process) can be performed for different time durations. The etching process (or any step of the etching process) can be performed for, for about, for at least, for at least about, for at most, or for at most about, 10 seconds (secs), 20 secs, 30 secs, 40 secs, 50 secs, 1 minutes (min), 2 mins, 3 mins, 4 mins, 5 mins, 6 mins, 7 mins, 8 mins, 9 mins, 10 mins, 11 mins, 12 mins, 13 mins, 14 mins, 15 mins, 16 mins, 17 mins, 18 mins, 19 mins, 20 mins, 21 mins, 22 mins, 23 mins, 24 mins, 25 mins, 26 mins, 27 mins, 28 mins, 29 mins, 30 mins, 31 mins, 32 mins, 33 mins, 34 mins, 35 mins, 36 mins, 37 mins, 38 mins, 39 mins, 40 mins, 41 mins, 42 mins, 43 mins, 44 mins, 45 mins, 46 mins, 47 mins, 48 mins, 49 mins, 50 mins, 51 mins, 52 mins, 53 mins, 54 mins, 55 mins, 56 mins, 57 mins, 58 mins, 59 mins, 60 mins, or a number or a range between any two of these values. For example, the etching process can be performed for about 1 min to about 10 mins.

The etching process (or any step of the etching process) can be performed at different temperatures in different embodiments. The etching process (or any step of the etching process) can be performed at a temperature of, of about, of at least, of at least about, of at most, of at most about, 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., or a number or a range between any two of these values. For example, the etching process can be performed at a temperature of about 1° C. to about 20° C.

The method can comprise removing the masking layer at the plurality of retained regions in contact with the plurality of particles which is differentially retained. In some embodiments, the plurality of etched regions can comprise no substance layer and no masking remaining after etching. Alternatively or additionally, the method can comprise passivating the plurality of etched regions to generate passivated regions (see FIG. 2A2 for an example). Passivating the plurality of etched regions can occur before removing the masking layer. The passivated regions can be hydrophilic, hydrophobic, positively charged, negatively charged, uncharged, or a combination thereof.

### Binding Sites

The planar structure and the plurality of particles in contact with the planar structure are subjected to etching, for example so as to recapitulate or mirror the particle distribution or configuration on the etched planar surface as binding sites. Referring to FIGS. 4A-4D, the planar structure after the etching process can include binding sites 404s1, 404s2. The planar structure can include a plurality of first regions 404r1 of binding sites 404s1 (corresponding to the particles in the first crystal lattice described with reference to FIGS. 3A-3D) and a plurality of second regions 404r2 of binding sites 404s2 (corresponding to the particles in the second crystal lattice described with reference to FIGS. 3A-3D). The plurality of first regions 404r1 of binding sites 404s1 and the plurality of second regions 404r2 of binding sites 404s2 are separated by a disjunction 406d. The binding sites 404s1, 404s2 in the plurality of first regions 404r1 of binding sites 404s1 and the plurality of second regions 404r2 are ordered, well packed, and have high density. A disjunction can include no binding site as illustrated in FIGS. 4A-4B. Alternately, a disjunction can include one or more binding sites 406i as illustrated in FIGS. 4C-4D. The binding sites in a disjunction are less ordered, less well packed, and/or have a lower density compared to binding sites 404s1, 404s2 in the plurality of first regions 404r1 of binding sites 404s1 and the plurality of second regions 404r2.

Referring to FIG. 4A, the pitch 422r1 (or the separation 420r1) of two, or any two, adjacent binding sites (or active bindings or reaction sites, or colonies on the binding sites) of the plurality of binding sites can be different in different embodiments. The pitch 422r1 of two adjacent binding sites (or active sites or reaction sites, or colonies on the binding sites) can be the distance between the centers of the two adjacent sites. The separation 420r1 of two adjacent binding sites can be the distance between the closest edges between the two adjacent binding sites. The pitch 422r1 (or the separation 420r1) of two, or any two, adjacent binding sites of the plurality of binding sites can be, be about, be at least, be at least about, be at most, or be at most about, 10 nanometer (nm), 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm, 1000 nm, 2 micrometer (μm), 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, or a number or a range between any two of these values. For example, the pitch of two, or any two, adjacent binding sites of the plurality of binding sites is about 1 nm to about 100 μm.

The methods disclosed herein can achieve binding sites with desired sizes. The desired size (e.g., radius 404r1r, diameter 404r1d, width, or height) of one, or one or more, or each, of the plurality of binding sites can be obtained. The size of one, or one or more, or each, of the plurality of binding sites can be, be about, be at least, be at least about, be at most, or be at most about, 10 nanometer (nm) 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm, 1000 nm, 2 micrometer (μm), 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, or a number or a range between any two of these values. For example, the size of one, or one or more, or each, of the plurality of binding sites is about 1 nm to about 100 μm.

Various binding site shapes are contemplated herein. For example, one, one or more, or each, of the plurality of binding sites has a circular or round shape. For example, one, one or more, or each, of the plurality of binding sites has an oval shape. For example, one, one or more, or each, of the plurality of binding sites has a rectangular shape. For example, one, one or more, or each, of the plurality of binding sites has a square shape. Other shapes are also consistent with the disclosure herein.

The methods disclosed herein can generate a high number of binding sites on each planar structure (such as a flow cell surface). The number of binding sites in the plurality of binding sites can be, be about, be at least, be at least about, be at most, or be at most about, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, 10,000,000, 20,000,000, 30,000,000, 40,000,000, 50,000,000, 60,000,000, 70,000,000, 80,000,000, 90,000,000, 100,000,000, 200,000,000, 300,000,000, 400,000,000, 500,000,000, 600,000,000, 700,000,000, 800,000,000, 900,000,000, 1,000,000,000, or a number or a range between any two of these values. For example, the number of binding sites in the plurality of binding sites is about 10,000 binding sites to about 10,000,000 binding sites.

### Binding Sites Configuration

The liquid from between the particles and the planar surface can be removed without substantial disruption of particle configuration in the liquid. The planar structure and the plurality of particles in contact with the planar structure are subjected to etching, for example so as to recapitulate or mirror the particle distribution or configuration on the etched planar surface as binding sites. Accordingly, the binding site configuration can mirror (e.g., the same or substantially the same) the particle configuration. For example, the binding sites can have (or can be described using or as having) a hexagonal configuration, an equilateral triangle configuration, a straight line configuration, or a linear configuration of the particles described with reference to FIGS. 3A-3D.

Referring to FIGS. 4A-4D, the plurality of first regions 404r1 can specify the plurality of binding sites (or active sites or reaction sites) 404s1 on the planar structure. The plurality of first regions 404r1 can be ordered as a first crystal lattice or in a first region of regular binding site array 402r1. The plurality of first regions 404r1 can comprise (or specify) the plurality of binding sites 404s1 on the planar structure. The plurality of second regions 404r2 can be ordered as a second crystal lattice or in a second region of regular binding site array 402r2. The plurality of second regions 404r2 can specify the plurality of binding sites 404s2 on the planar structure. The plurality of first regions 404r1 (and the binding sites 404s1 specified) can be separated from the plurality of second regions 404r2 (and the binding sites 404s2 specified) by a disjunction 406d.

Referring to FIG. 4A, the binding sites can have (or can be described using or as having) a hexagonal configuration 408h1. The plurality of first regions can comprise a first subset of seven first binding sites of the plurality of binding sites configured into six vertices and a center of a first hexagon 410h1. The plurality of second regions 404r2 can comprise (or specify) a second subset of seven second binding sites of the plurality of binding sites configured into six vertices and the center of a second hexagon 410h1. The first hexagon 410h1 and the second hexagon 410h2 can share no side. The first hexagon 410h1 and the second hexagon 410h2 can have an identical orientation such that the first hexagon and the second hexagon are related by translation, no rotation. The first hexagon 410h1 and the second hexagon 410h2 can have different orientations such that the first hexagon and the second hexagon are related by a rotation (and a translation) as illustrated in FIG. 4A. The hexagons in a hexagonal configuration can share sides. The hexagons in different hexagonal configurations may not share sides. The hexagons in a hexagonal configuration can be congruent (e.g., having the same size and shape). The hexagons in different hexagonal configurations can be congruent (e.g., having the same size and shape) or different. The hexagons in different hexagonal configuration can be related by a rotation (and a translation). The hexagons in different hexagonal configurations can be related by a translation (not a rotation) such that the hexagons have the same orientation. The binding sites having a hexagonal configuration can be tightly packed, well separated, and ordered in a non-predetermined manner generated.

Referring to FIG. 4B, the binding sites can have (or can be described using or as having) an equilateral triangle configuration 408t1, 408t2. The plurality of first regions 404r1 can comprise (or specify) a first subset of three first binding sites of the plurality of binding sites 404s1 configured into three vertices of a first equilateral triangle 410t1. The plurality of second regions 404r2 can comprise a second subset of three second binding sites of the plurality of binding sites 404s1 configured into three vertices of a second equilateral triangle 410t1. The first equilateral triangle 410t1 and the second equilateral triangle 410t2 can share no side. The first equilateral triangle 410t1 and the second equilateral triangle 410t2 can be congruent (e.g., having the same size and shape) as illustrated in FIG. 4B. The first equilateral triangle 410t1 and the second equilateral triangle 410t2 can have an identical orientation such that the first equilateral triangle and the second equilateral triangle are related by translation (no rotation). The first equilateral triangle 410t1 and the second equilateral triangle 410t2 can be related by a rotation (and a translation). The first equilateral triangle 410t1 and the second equilateral triangle 410t2 can have different orientations as illustrated in FIG. 4B. The equilateral triangles in an equilateral triangle configuration can share sides. The equilateral triangles in different equilateral triangle configurations may not share sides. The equilateral triangles in an equilateral triangle configuration can be congruent (e.g., having the same size and shape). An equilateral triangle configuration can include two subsets of equilateral triangles. Equilateral triangles in each subset can be related by translation (not rotation). Equilateral triangles between the two subsets are related by a rotation of 180° (and a translation). The equilateral triangles in different equilateral triangles configurations can be congruent (e.g., having the same size and shape) or different. The equilateral triangle in different equilateral triangles configurations can be related by a rotation (and a translation) as illustrated in FIG. 4B. The equilateral triangles in different equilateral triangles configurations can be related by a translation (not a rotation), such that the equilateral triangles have the same orientation for corresponding subsets of equilateral triangles. The particles having an equilateral triangle configuration can be tightly packed, well separated, and ordered in a non-predetermined manner.

Referring to FIG. 4C, the binding sites can have (or can be described using or as having) a straight line configuration 408s1, 408s2. The plurality of first regions 404r1 on the planar structure after the etching process can comprise two adjacent first binding sites of the plurality of binding sites 404s1. The plurality of second regions 404r2 can comprise two adjacent second binding sites of the plurality of binding sites 404s2. A first straight line 410s1 drawn between the two adjacent first binding sites and a second straight line 410s2 drawn between the adjacent second binding sites have an identical direction/are parallel. The first straight line 410s1 and the second straight line 410s2 can intersect at an angle that is an integer multiple of 60°. The first straight line 410s1 and the second straight line 410s2 can have different directions. The first straight line and the second straight line can be related by a rotation (and a translation) other than a rotation of an integer multiple of 60°. The straight lines in different straight line configurations can be parallel such that the straight lines are related by a rotation (and a translation). The straight lines in different straight line configurations can be related by a translation (not a rotation), such that the straight lines have the same direction. The particles having a straight line configuration can be tightly packed, well separated, and ordered in a non-predetermined manner.

Referring to FIG. 4D, the binding sites can have (or can be described using or as having) a linear configuration. The plurality of first regions 404r1 can comprise first binding sites of the plurality of binding sites 404s1 arranged in a plurality of first rows 410l1 of first binding sites. First binding sites 404s1 in each first row of the plurality of first rows 410l1 can be arranged in a first linear configuration 408l1 such that a first binding site in the first row is in contact with two first binding sites adjacent to the first binding site in the first row. Each first binding site other than the first binding sites at the end of a first row is in contact with two first binding sites adjacent to the first binding site in the first row. Two adjacent first rows 410l1 can be offset by a first offset 412o1 of the first linear configuration. in a first direction 414d1 of the first linear configuration. The offset 412o1 can be more than a radius and less than a diameter of a particle of the plurality of particles, such as the square root of three multiplied by the radius of the particle. The two adjacent first rows 410l1 can be offset by a second offset 416o1 of the first linear configuration, for example the diameter of the particle of the plurality of particles, in a second direction 418d1 of the first linear configuration. The second direction 418d1 of the first linear configuration can be perpendicular to the first direction 414d1 of first linear configuration. A first particle in one first row can be in contact with two adjacent first particles in an adjacent first row.

The plurality of second regions 404r2 can comprise second binding sites 404s2 of the plurality of binding sites arranged in a plurality of second rows 410l2 of second binding sites. Second binding sites 404s2 in each second row of the plurality of second rows 410l2 can be arranged in a second linear configuration 408l2 such that a second binding site in the second row is in contact with two second binding sites adjacent to the second binding site in the second row. Each second binding site other than the second binding sites at the end of a second row is in contact with two second binding sites adjacent to the second binding site in the second row. Two adjacent second rows 410l2 can be offset by a first offset 412o2 of the second linear configuration in a first direction 424d2 of the second linear configuration. The offset 412o2 can be more than a radius and less than a diameter of a particle of the plurality of particles, such as the square root of three multiplied by the radius of the particle. The two adjacent second rows can be offset by a second offset 416o2 of the second linear configuration, for example a diameter of the particle of the plurality of particles, in a second direction 418d2 of the second linear configuration. The second direction 418d2 of the second linear configuration can be perpendicular to the first direction 414d2 of the second linear configuration. A second particle in one second row can be in contact with two adjacent second particles in an adjacent second row. The particles having a linear configuration can be tightly packed, well separated, and ordered in a non-predetermined manner.

The first offset 412o1 of the first linear configuration and the first offset 412o2 of the second linear configuration can be identical, or within a percentage of each other. The second offset 416o1 of the first linear configuration and the second offset 416o2 of the second linear configuration can be identical, or within a percentage or more of each other. The percentage can be different in different embodiments. The percentage can be, be about, be at least, be at most, or be at most about, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values.

One or more parameters described with reference to the first linear configuration 408l1 and the second linear configuration 408l2 can be the same or different. The first linear configuration 408l1 and the second linear configuration 408l2 can be different (or the same), for example, with respect to some or all of the parameters described with reference to the linear configurations. The orientations of the linear configurations can be different (or the same). The first direction 414d1 of the first linear configuration and the first direction 414d2 of the second linear configuration can be different (or the same). The second direction 418d1 of the first linear configuration and the second direction 418d2 of the second linear configuration can be different (or the same). The combination of the first direction 414d1 and the second direction 418d1 of the first linear configuration and the combination of the first direction 414d2 and the second direction 418d2 of the second linear configuration can be different (or the same). For example, the first angle formed by the intersection of the first direction 414d1 and the second direction 418d1 of the first linear configuration and the second angle formed by the intersection of the first direction 414d2 and the second direction 418d2 of the second linear configuration are related to each other by a rotation (and a translation).

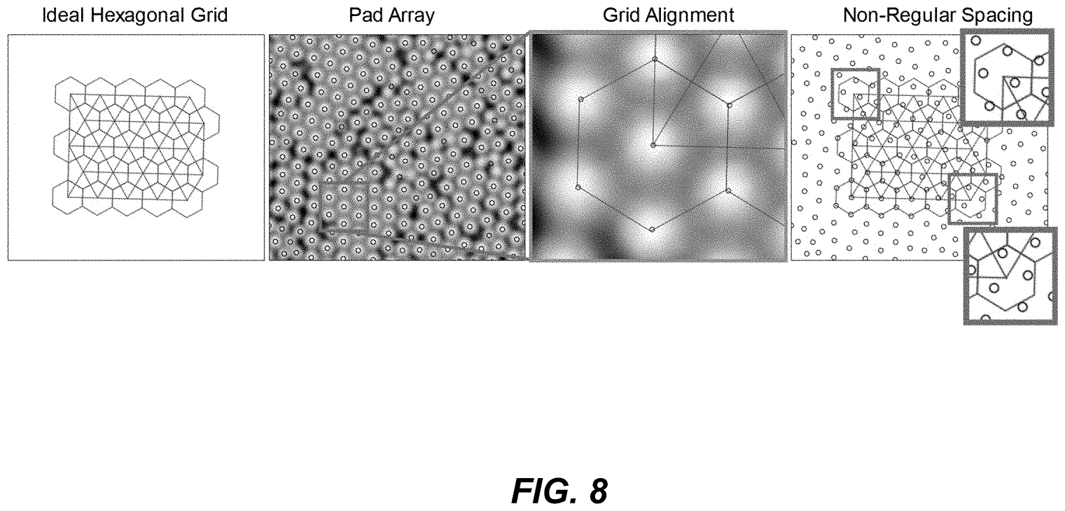

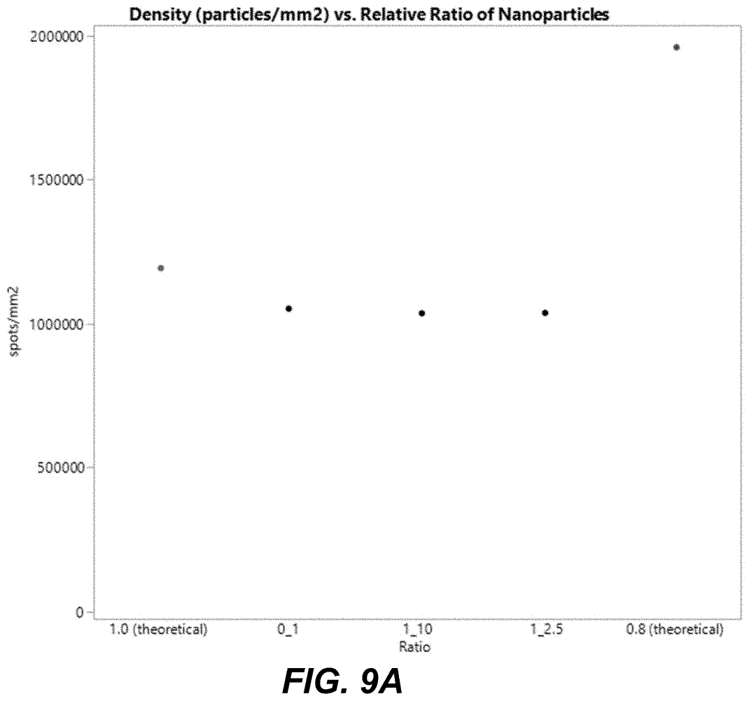

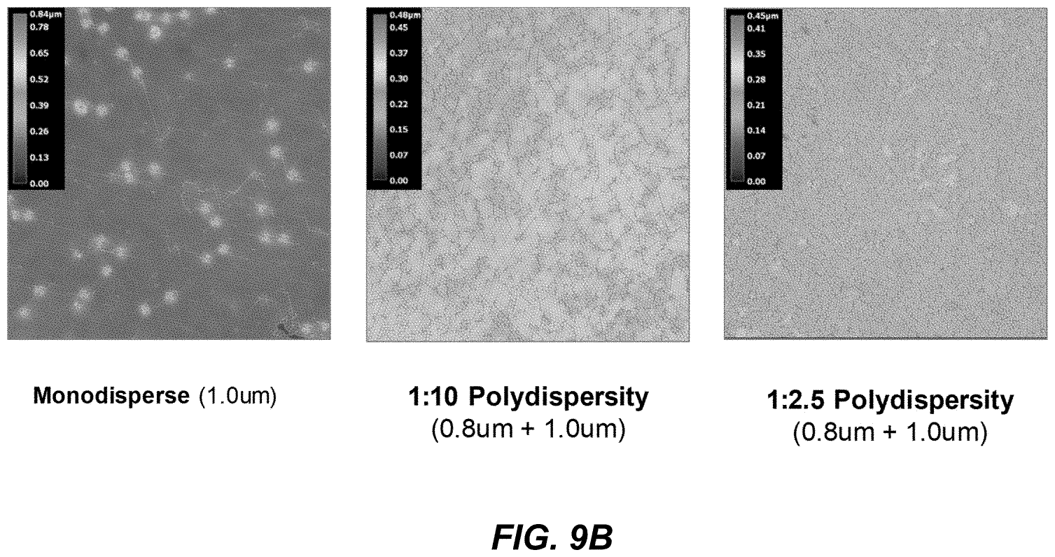

The plurality of binding sites can comprise a subset of binding sites of the plurality of binding sites in a crystal lattice or an irregular array. An irregular array of binding sites can be generated with particles with polydispersity (e.g., particles with two different sizes). An irregular array of binding sites can be generated when an irregular array of particles form at the surface of the liquid (or an irregular array of particles is deposited on the planar structure) prior to the etching step. An irregular array can result from, for example, a mixture of particles with two different sizes being present on the surface of the liquid and/or being deposited onto the planar structure. FIG. 8 shows the binding sites generated using beads with 0.8 μm diameter and beads with 1.0 μm diameter at a ratio of 2:5 respectively (by the number of beads). The binding sites are irregularly arranged, meaning the binding sites have non-regular spacing. As illustrated, an ideal hexagonal grid (FIG. 8, far left panel) does not align with the binding sites or the centers of the binding sites (FIG. 8, far right panel). The ideal hexagonal grid represents a hexagonal configuration. FIG. 9A shows that density of the spots are not significantly sensitive to disorder. Disordered surface shows at most a 10-15% reduction from perfectly ordered arrays. FIG. 9B shows array irregularities resulting from monodispersity with 1.0 μm-diameter beads, polydispersity with 0.8 μm-diameter beads and 1.0 μm beads at 1:10 ratio, and polydispersity with 0.8 μm-diameter beads and 1.0 μm beads at 2:5 ratio.

The plurality of binding sites can comprise two subsets (or populations) of binding sites of the plurality of binding sites in crystal lattices (e.g., a first crystal lattice and a second crystal lattice) or irregular arrays (e.g., a first irregular array and a second irregular array). The two crystal lattices (or irregular arrays) can be separated by a disjunction. A crystal lattice and an irregular array can be separated by a disjunction. In some embodiments, a crystal lattice is in a hexagonal configuration, an equilateral triangle configuration, a straight line configuration, a linear configuration, or a combination thereof. Binding sites not being in a hexagonal configuration means that a hexagonal grid (e.g., a hexagonal grid shown in FIG. 4A) cannot be aligned with the binding sites. Binding sites not being in an equilateral triangle configuration means that a triangle grid (e.g., a triangle grid shown in FIG. 4B) cannot be aligned with the binding sites. Binding sites not being in a linear configuration means that a linear grid (e.g., a linear grid shown in FIG. 4C) cannot be aligned with the binding sites. Binding sites not being in a straight line configuration means that a straight line grid (e.g., a straight line grid shown in FIG. 4D) cannot be aligned with the particles. In some embodiments, an irregular array comprises no binding sites in a hexagonal configuration, an equilateral triangle configuration, a straight line configuration, a linear configuration, or a combination thereof. An irregular array can comprise no particles within at least a threshold distance of each other that are in a hexagonal configuration, an equilateral triangle configuration, a straight line configuration, a linear configuration, or a combination thereof. The threshold distance can be, be about, be at least, be at least about, be at most, or be at most about, 0.1 μm, 0.2 μm, 0.3 μm, 0.4 μm, 0.5 μm, 0.6 μm, 0.7 μm, 0.8 μm, 0.9 μm, 1 μm, 1.1 μm, 1.2 μm, 1.3 μm, 1.4 μm, 1.5 μm, 1.6 μm, 1.7 μm, 1.8 μm, 1.9 μm, 2 μm, 3 μm, 4 μm, 5 μm, 6 μm, 7 μm, 8 μm, 9 μm, 10 μm, or a number or a range between any two of these values. Two flow cell surfaces can share a congruent binding site configuration only if the configuration comprises at most 1%, 2%, 3%, 4%, 5%, 6%, 7%, 8%, 9%, 10%, or more of the binding sites on each of the two flow cell surfaces.

Pitches between one binding site and two neighbor (or adjacent) binding sites can be different. For example, three consecutive binding sites in a straight (or substantially straight) line can have different pitches between the two pairs of consecutive binding sites. A pitch between the middle binding site and one neighbor binding site and a pitch between the middle binding site and another neighbor binding site can be different. For example, a binding site can have a number of neighbor binding site (e.g., 2, 3, 4, 5, or 6 neighbor binding sites). The pitches between the binding site and 2 (or 3, 4, 5, or 6) neighbor binding sites can differ by, by about, by at least, by at least about, by at most, or by at most about, for example, 0.01 μm, 0.02 μm, 0.03 μm, 0.04 μm, 0.05 μm, 0.06 μm, 0.07 μm, 0.08 μm, 0.09 μm, 0.1 μm, 0.11 μm, 0.12 μm, 0.13 μm, 0.14 μm, 0.15 μm, 0.16 μm, 0.17 μm, 0.18 μm, 0.19 μm, 0.2 μm, 0.3 μm, 0.4 μm, 0.5 μm, 0.6 μm, 0.7 μm, 0.8 μm, 0.9 μm, 1 μm, or a number or a range between any two of these values.

A flow cell surface provided herein can comprise a plurality of binding sites separated by disjunctions. The binding sites and/or the disjunctions can be at positions that are not predetermined. The binding sites and/or the disjunctions can be ordered, can be irregularly distributed, and/or can be randomly distributed. In some instances, configurations of the binding sites can be not predetermined. The disjunctions can be at positions that are not predetermined. The disjunctions (including binding sites therein if any) can be irregularly distributed and/or can be randomly distributed. Binding sites of the plurality of binding sites can be at positions that are not predetermined. The binding sites can be ordered, irregularly distributed (or arranged), and/or randomly distributed (or arranged). In some instances, the plurality of binding site can comprise a first plurality of binding sites and a second plurality of binding sites separated by a disjunction. The position, size, and/or shape of the disjunction can be not predetermined. The disjunction can be randomly distributed. Binding sites of the first plurality of binding sites and/or the second plurality of binding sites can be at positions that are not predetermined, can be ordered, can be irregularly distributed, and/or can be randomly distributed. A subset of binding sites can have a configuration that is not predetermined. A configuration can comprise the number binding sites in the subset of binding sites and/or positions of the binding sites in the subset of binding sites. The first subset of binding sties and the second subset of binding sites can have configurations that are different (or identical).

### Disorder

There is a principal distinction between the colloidal process described herein and conventional top-down nanofabrication processes regarding order in the resulting structures. Despite enabling almost maximum packing density on a surface and demonstrating illustrative order, a mathematically generated grid or lattice cannot be fitted on the spots pattern on the surface generated using the process described herein, and coordinates of any spot on the surface cannot be extrapolated according to an origin in the lattice. FIG. 8 shows the process of overlaying a mathematically generated coordinates grid depicting a hexagonally close-packed lattice with lattice constant of 1 μm on a Scanning Electron Microscopy (SEM) image of part of a crystal with presumed high crystallinity and order to evaluate the degree of order in the crystal microstructure. The crystalline array was self-assembled using 1 μm polystyrene microspheres as assumed for mathematical model. The crystalline array of pads and the ideal hexagonal grid exhibit significant mismatch even in the range of about 5 μm away from where is assumed as origin for both grids. The inherent randomness associated with bottom-up fabrication strategies such as self-assembly dictates the presence of defects in resulting crystalline structures which deviate from any predicted long or short-range order.

To control density of DNA clusters on a surface, the order of the pads can be tuned. To deviate from long range and highly ordered crystals, density of defects including vacancies, lattice mismatch, dislocations, and grain boundaries must increase, which will directly impact the size of individual crystals as well as the structure and order of the entire sample. There are different strategies to increase defects and imperfections in a crystal:

(1) A binary mixture of polystyrene micro/nanospheres (1 μm and 800 nm polystyrene beads) instead of a monodispersed suspension of beads (only 1 μm microspheres) used for self-assembly in the crystallization process, results in a high density of defects and consequently a highly disordered crystalline structure. Increasing the concentration of substituting beads (800 nm nanospheres) inhibits long-range order and yield smaller crystals.

(2) Increasing the concentration of spreading agents including ethanol and isobutanol in the bead suspension interferes with self-assembly process of beads in the liquid-air interface. This interference is manifested in form of a fine crystalline structure with very small crystals with a wide range of sizes.

(3) Self-assembly of beads can also be interfered by dissolving surfactants (sodium dodecyl sulfate and Tween), polyethylene glycol, sucrose, urea and polyacrylic acid in the subphase. These reagents act as contaminants which impede long range self-assembly and crystallization. Tuning the pH of the bath with stronger acids and bases also affects bead transfer in the liquid-air interface which changes the scale of order in the subsequent crystals.

(4) Minimal disturbance of liquid-air interface during self-assembly and crystallization is a principal requirement for achieving long-range order. Hence, controlled disruption of the crystallization wave-front can govern the density of defects in the crystals. Having multiple injection points (instead of one).

### Binding Site Layer

The planar structure, the binding site layer, and/or the masking layer is often uniform (or substantially uniform) prior to any step of the method, such as the depositing the plurality of beads and the etching process. The planar structure can be uniform prior to the depositing the plurality of beads or the etching process. The binding site layer (or substance layer, reaction site layer, or active site layer) can uniform prior to the depositing the plurality of beads and the etching process. The masking layer can be uniform prior to the depositing the plurality of beads or the etching process.

### Binding Site Functionalization

The binding site layer (or substance layer, reaction site layer, or active site layer) can functionalize the planar structure. The functionalized planar structure can be used for sequencing, including next generation sequencing. For example, the binding sites can comprise aminosilane for capturing of emPCR beads. The binding site layer can comprise an acrylate functional silane, an aldehyde functional silane, an amino functional silane, an anhydride functional silane, an azide functional silane, a carboxylate functional silane, a phosphonate functional silane, a sulfonate functional silane, an epoxy functional silane, a thiol functional silane, an ester functional silane, a vinyl functional silane, an olefin functional silane, a halogen functional silane, a dipodal silane, or a combination thereof. In some embodiments, the binding site layer comprises an aminosilane, a glycidoxysilane, a mercaptosilanes, or a combination thereof. An aminosilane can be (3-aminopropyl)triethoxysilane, (3-aminopropyl)-diethoxy-methylsilane, (3-aminopropyl)-dimethyl-ethoxysilane, or (3-aminopropyl)-trimethoxysilane. A glycidoxysilane can be (3-glycidoxypropyl)-dimethyl-ethoxysilane. A mercaptosilane can be (3-mercaptopropyl)-trimethoxysilane or (3-mercaptopropyl)-methyl-dimethoxysilane. In some embodiments, the binding site layer comprises an aminosilane. The binding site layer can be hydrophilic, hydrophobic, positively charged, negatively charged, uncharged, or a combination thereof. The binding site layer can comprise a functional moiety for covalent attachment, such as a functional moiety capable of participating in the click chemistry reaction. The binding site layer can be used to attach a biomolecule to binding sites of a structured surface, where the biomolecule itself binds to a DNA tile, concatemer, or other nucleic acid. The biomolecule may be an affinity biomolecule that specifically binds to an affinity partner present on the DNA tile, concatemer or other nucleic acid. For example, the biomolecule could be biotin where the affinity partner is avidin (e.g., streptavidin), or the biomolecule could be avidin (e.g., streptavidin) where the affinity partner is biotin. Alternatively, the biomolecule may be an oligonucleotide that specifically hybridizes to the DNA tile, concatemer, or other nucleic acid. For example, the oligonucleotide may be a primer, such as a splint primer that allows for rolling circle amplification from the surface thereby forming a covalently attached concatemer. The binding sites may bind to an intermediate nucleic acid, such as a DNA tile, that in turn binds to the nucleic acid (e.g., to a nucleic acid to be sequenced, such as a concatemer). The intermediate nucleic acid may have one or more attachment points to the binding site, and may be bound to the binding site by affinity, hybridization, or covalently. The intermediate nucleic acid may hybridize to the nucleic acid to be sequenced, or may present a functional group that binds to the nucleic acid to be sequenced. The surface chemistry described herein may be used to attach such a biomolecule to binding sites of a structured surface. As such, flow cells of the subject application may comprise binding sites with one or a combination of the above characteristics.

Covalent Surface Chemistry

To capture and immobilize DNA clusters on the surface, such as binding sites on a structured surface, complementary functional groups on the surface and the clusters can be used to enable covalent attachment between them. Chemical Vapor Deposition (CVD), such as Plasma-Enhanced Chemical Vapor Deposition (PECVD) may be used to graft hyperbranched polymeric thin film with non-fouling back bone. Monomer solution including alcohols, glycidol, and glycidyl ethers are vaporized at elevated temperatures (50-150° C.) and low pressures (10-200 mTorr) in a bubbler and the vapor is injected into a chamber as precursor. A plasma environment is then generated from the precursor by applying Radio Frequency (RF) in continuous and pulsed forms. By tuning parameters that govern plasma conditions including RF source (continuous vs pulsed), RF power, pulsing frequency, process pressure, precursor concentration, temperature and flowrate, and substrate temperature, polymerization and end functional groups on the polymeric thin film can be controlled. Functional groups including hydroxyl, carboxyl, amine, and epoxide are obtained directly from PECVD process. The functional surface attained by PECVD is converted to other functional groups including MTZ, TCO, thiol, maleamide, azide, and DBCO through bath conversion chemistry including substitution of tosylate, mesylate, and triflate groups. A post gas-phase treatment of the polymeric thin film is also used to alter functional groups, for example using epichlorohydrin. Alternatively, a directly functional capture surface may emerge from the CVD/PECVD process. In another approach, surface functional groups are grafted onto the substrate surface using silane chemistry. Functional silane precursors are reacted with activated silanols on glass substrates and provide functional groups on the surface. Both gas-phase Chemical Vapor Deposition (CVD) and bath processes are used for silane chemistry. The CVD process is very similar to what has been described about the PECVD process. A precursor (typically a silane-spacer-functional group or a monomer solution) is introduced into a chamber where the samples are placed, in vapor form at low pressures (30-250 mTorr) and high temperatures (50-250° C.). The desired functional moieties are grafted onto the surface with short polymeric backbones. The functional group density on the surface is tailored by adjusting process parameters to tune covalent DNA cluster capture and immobilization on the substrate surface.

A solid surface may be prepared to enable covalent capture of biomolecules of interest by a variety of approaches, hereafter referred to as a capture surface, including installing a “click” chemistry reaction partner on the capture surface, installing moieties with inducible reactivity, e.g. photo-activated capture moieties, or these approaches in combination with a feature that ensures close proximity of the biomolecule of interest to the reactive moiety for covalent capture, e.g. a complementary DNA capture probe or an affinity interaction partner such as antibody interactions or the biotin-avidin interaction. If a biomolecule of interest is labelled with a click chemistry moiety then these groups may be specifically reacted with a capture surface that has the conjugate reaction partner. Alternatively, a capture surface may capture biomolecules non-specifically via photo-induced crosslinking agents, such as psoralen, or chemically inducible crosslinking agents such as a pendant furan oxidized in the presence of an appropriate oxidizing agent such as N-bromosuccinimide. The efficiency of these non-specific crosslinking reactions may be improved by ensuring proximity of the biomolecule of interest when crosslinking is induced via, for example, base-pairing of a complement DNA, extensive hydrogen bonding, and/or an affinity binding interaction. These capture surfaces may be prepared for covalent by several coating methods, including dip coating, chemical vapor deposition, plasma-enhanced chemical vapor deposition, and/or grafting from or grafting to of appropriately functionalized polymers. Exemplary diagrams of a surface prepared by silane dip coating can be seen in 3 in the attached figures and an exemplary diagram of a thin plasma polymerized hydrogel surface can be seen in 4 in the attached figures, both functionalized with a representative click chemistry reaction partner, methyltetrazine.

### Interstitial Chemistry

After the etching step, the polymer masking layer on the area between the polystyrene beads also known as interstitials is removed and underlying bare glass is exposed. To minimize the unfavorable interaction between DNA and glass, the interstitials may be coated with a thin polymeric film using PECVD. Monomers including alcohols, glycidol, and glycidyl ethers are used to graft a crosslinked thin film on glass. Process parameters are tuned to obtain hydrophilic functional groups including hydroxyl and carboxyl. By tuning PECVD process parameter, other functional groups can be retained to keep the interstitials as an active surface. This fabrication strategy enables differential surface chemistry on structured surfaces. Alternative coatings, for example inorganic coatings, may be useds in place of a thin polymeric film.

To further exploit binary surface chemistry, after a surface such as bare glass is coated with the masking layer and etched, the desired passivating chemistry (e.g., groups that prevent binding of nucleic acid, such as a polymer) are grafted on the interstitials using gas-phase CVD and PECVD processes while the beads prevent grafting to the future binding sites. After interstitial functionalization followed by removing polymeric masking layer and exposing bare glass, a secondary CVD process is used to graft the active chemistry required for DNA covalent capture on structured pads. While the interstitials are designed to be passive, further bath functionalization can be used to convert the interstitials into chemically active moieties for other chemical processes. In general, the interstitial space may be bound to a polymer, such as a hydrophilic polymer such as PEG, that prevents binding of nucleic acids by this or another process.

### Nucleic Acids

The plurality of binding sites can be used for next generation sequencing, such as sequencing-by-synthesis or sequencing-by-binding. As such, the methods herein may further include sequencing of nucleic acids, for example concatemers such as RCA products that are bound directly or indirectly to a surface as described herein. The methods of the present disclosure can include delivering a plurality of nucleic acids to the plurality of binding sites. Each of the plurality of nucleic acids can be delivered to a different binding site of the plurality of binding sites. The plurality of nucleic acids comprises at least one concatemer (or concatemeric nucleic acid). The plurality of nucleic acid can be a DNA origami. The DNA origami can be a DNA tile. The plurality of nucleic acids can be distributed on a plurality of beads, such as emulsion PCR beads. Each of the plurality of nucleic acids can be distributed to a different bead of the plurality of beads.

The use of a concatemeric nucleic acid for next generation has been described in U.S. Provisional Application No. 62/984,438, entitled “METHODS AND COMPOSITIONS FOR SEQUENCING DOUBLE STRANDED NUCLEIC ACIDS,” filed Mar. 3, 2020, the content of which is incorporated herein by reference in its entirety. Briefly, the term “concatemer,” when used in reference to a nucleic acid molecule, means a continuous nucleic acid molecule that contains multiple copies of a common sequence linked in series. Similarly, the term “concatemer,” when used in reference to a nucleotide sequence, means a continuous nucleotide sequence that contains multiple copies of a common sequence in series. Each copy of the sequence can be referred to as a “sequence unit” of the concatemer. A sequence unit can have a length of at least 10 bases, 50 bases, 100 bases, 250 bases, 500 bases or more. A concatemer can include at least 2, 5, 10, 50, 100 or more sequence units. A sequence unit can include subregions having any of a variety of functions such as a primer binding region, target sequence region, tag region, unique molecular identifier (UMI), or the like. A method of performing next generation sequencing using a concatemer can include (a) providing a nucleic acid cluster attached to a solid support, wherein the nucleic acid cluster includes a sense strand of a concatemer and an antisense strand of the concatemer, wherein the concatemer includes multiple copies of a sequence unit linked in series, wherein the sequence unit includes a target sequence and a primer binding site. The method can include (b) hybridizing a primer to the primer binding site in a sequence unit of the antisense strand in the cluster; and (c) extending the primer along the antisense strand to determine the sequence from at least a portion of the target sequence in the antisense strand. Additionally, the method can include (d) hybridizing a second primer to the primer binding site in a sequence unit of the sense strand; and (e) extending the second primer along the sense strand to determine the sequence from at least a portion of the target sequence in the sense strand. A concatemer may comprise a sense strand, optionally hybridized to one or more antisense strands. For example, when rolling circle amplification is used to produce a concatemer (i.e., having a sense strand), primers may be hybridized to the concatemer and extended in a multiple strand displacement amplification that results in a concatemer comprising multiple antisense strands that are at least in part hybridized to the sense strand.

The percentage of the plurality of binding sites comprising at least one nucleic acid of the plurality of nucleic acids (or one bead of the plurality of beads) can be different in different embodiments. The percentage of the plurality of binding sites comprising at least one nucleic acid of the plurality of nucleic acids (or one bead of the plurality of beads) is, is about, is at least, is at least about, is at most, or is at most about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. For example, at least 50% of the plurality of binding sites comprises at least one nucleic acid of the plurality of nucleic acids and/or one bead of the plurality of beads.

Next generation sequencing can include performing colony formation such as bridge amplification or rolling circle amplification at the plurality of binding sites. Next generation sequencing can include delivering an excitation energy to at least some of the binding sites. Next generation sequencing can include collecting an emission energy from at least some of the binding sites. Although the applications of the binding sites are described with reference to next generation sequencing, the reference is illustrative only and is not intended to be limiting.

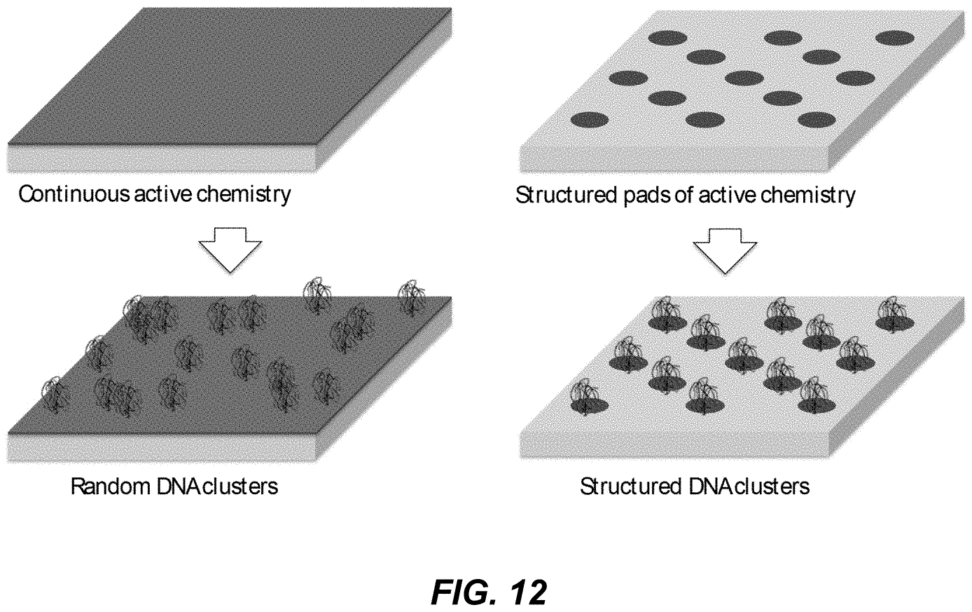

Alternatively or additionally, rolling circle amplification (RCA) can be performed in solution or at the plurality of sites. For example, rolling circle amplification can be performed at the plurality of binding sites using an amplification primer (or a splint primer) attached to one, one or more, or each of the plurality binding sites (see FIG. 12 for illustration). FIG. 12 compares rolling circle amplification (RCA) at binding sites with active chemistry (e.g., with amplification primers attached to the binding sites) and randomly on a flow cell surface with active chemistry. The amplification products formed on the binding sites can be regularly or irregularly arrayed. When RCA is performed in solution, the resulting concatemer (RCA product) may later be bound to binding sites as described herein.

The amplification primer (or the splint primer) can be attached to the binding site. The amplification primer (or the splint primer) can comprise a first functional moiety capable of participating in a click chemistry reaction. The first functional moiety can comprise, for example, methyltetrazine (MTz). The substance layer can comprise a second functional moiety capable of participating in the click chemistry reaction. The second functional moiety can comprise trans-cyclooctene (TCO). The amplification primer or the splint primer can be attached to the binding site via the click chemistry reaction involving the first functional moiety and the second functional moiety. Rolling circle products may be covalently attached to a solid surface (e.g., biding sites of a flow cell described herein) for purposes of sequencing in a variety of fashions. A highly processive DNA polymerase, Phi29, is commonly used for amplification in rolling circle amplification and it is known to be somewhat permissive to incorporation of base-modified non-natural nucleotides during amplification. A mixture of natural nucleotides and nucleotides with base modifications included in the amplification reaction will render a rolling circle product with some random incorporation of the modified nucleotides throughout the structure of the rolling circle product. If the nucleotide modifications are, for example, a moiety belonging to the family of click chemistry reactions, e.g. azide, strained unsaturated rings such as trans-cyclooctene (TCO) or dibenzocyclooctyne (DBCO), any alkene or alkyne, thiol, etc., then these pendant modifications from random bases within the rolling circle product can be used to react with a solid surface that is appropriately conjugated. Alternatively, rolling circle product could be similarly modified with base modified nucleotides that enable covalent attachment by employing the unusual activity of some polymerases, famously including terminal deoxynuleotidyl transferase (TdT), for non-templated 3′ extension. TdT is known to be very permissive in the nucleotide modifications it will incorporate on a free 3′ end of DNA. The nucleotide modifications of interest are the same as above, e.g. azide, strained unsaturated rings such as trans-cyclooctene (TCO) or dibenzocyclooctyne (DBCO), any alkene or alkyne, thiol, etc. This would result in modified nucleotides being incorporated exclusively at the 3′ end of the rolling circle product. These modified nucleotides could be covalently immobilized on any surface prepared with the conjugate reaction partner for the given reactive group. As such, RCA can be used to incorporate nucletiodes functionalized to bind to the binding sites of flow cells described herein, such that the resulting concatemer can be deposited.

In some embodiments, the click chemistry reaction comprises copper catalyzed azide-alkyne cycloaddition (CuAAC). The covalent linkage can comprise a triazolyl. The CuAAC can comprise a Cu(I) stabilizing ligand. The Cu(I) stabilizing ligand can be selected from the group consisting of: 3-[4-({bis[(1-tert-butyl-1H-1,2,3-triazol-4-yl)methyl]amino}methyl)-1H-1,2,3-triazol-1-yl]propanol (BTTP), 3-[4-({bis[(1-tert-butyl-1H-1,2,3-triazol-4-yl)methyl]amino}methyl)-1H-1,2,3-triazol-1-yl]propyl hydrogen sulfate (BTTPS), 2-[4-({bis[(1-tert-butyl-1H-1,2,3-triazol-4-yl)methyl]amino}methyl)-1H-1,2,3-triazol-1-yl]ethyl hydrogen sulfate (BTTES), 2-[4-{(bis[(1-tert-butyl-1H-1,2,3-triazol-4-yl)methyl]amino)methyl}-1H-1,2,3-triazol-1-yl]-acetic acid (BTTAA), bathophenanthroline disulfonate disodium salt (BPS), N,N,N′,N″,N″-Pentamethyldiethylenetriamine (PMDETA), tris-((1-benzyl-1H-1,2,3-triazol-4-yl)methyl)amine (TBTA), Tris(3-hydroxypropyltriazolylmethyl)amine (THPTA), NF-((1R,2R)-2-azidocyclopentyloxy)carbonyl)-L-lysine (ACPK), and 4-N,N-dimethyl amino-1,8-naphthalimide (4-DMN).

In some embodiments, the click chemistry reaction comprises strain-promoted azide-alkyne cycloaddition (SPAAC). The covalent linkage can comprise a cycloocta-triazolyl. In some embodiments, the click chemistry reaction comprises alkyne hydrothiolation. The covalent linkage can comprise an alkenyl sulfide. In some embodiments, the click chemistry reaction comprises alkene hydrothiolation. The covalent linkage can comprise an alkyl sulfide. In some embodiments, the click chemistry reaction comprises strain-promoted alkyne-nitrone cycloaddition (SPANC). The covalent linkage can comprise an octahydrocycloocta-isoxazolyl. The cyclooctynyl can be dibenzylcyclooctyne (DBCO) or a derivative thereof. In some embodiments, the click chemistry reaction is biocompatible.

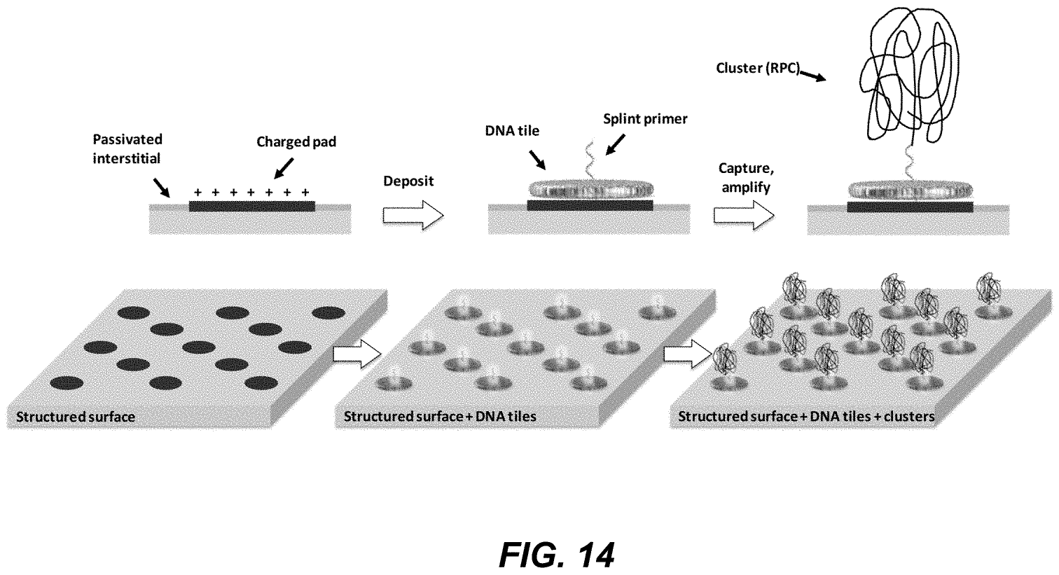

In some embodiments, a plurality of DNA tiles can be delivered to the plurality of binding site (see FIG. 14 for an illustration). Each of the plurality of binding sites can comprise at most one DNA tile of the plurality of DNA tiles. Two binding sites of the plurality of binding sites can comprise two different DNA tiles of the plurality of DNA tiles. One or more DNA tiles of the plurality of DNA tiles each can comprise an amplification primer (or a splint primer). The method can further comprise performing rolling circle amplification (RCA) at the plurality of binding sites by extending the amplification primer (or the splint primer) attached to each of the two or more DNA tiles using a plurality of template nucleic acids as templates. The rolling circle amplification can be performed at the binding sites.

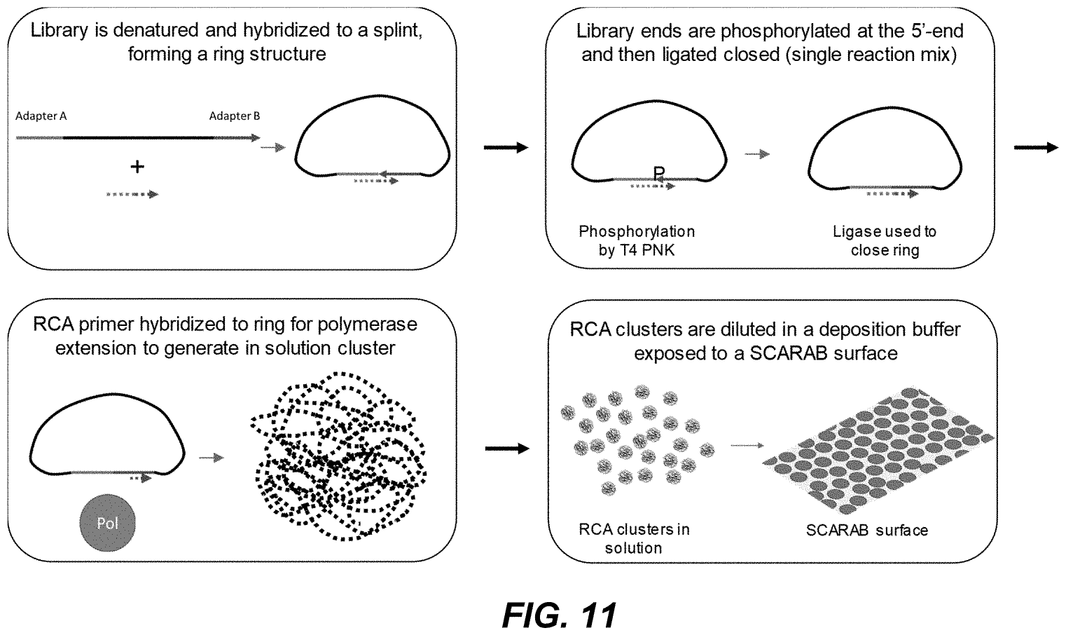

Alternatively or additionally, rolling circle amplification (RCA) can be performed in solution (see FIG. 11 for an illustration) and amplification products are delivered to the plurality of binding sites (see FIG. 12 for an illustration). In certain aspects, RCA may incorporate nucleotides functionalized to bind to the binding sites. Such nucleotide may be functionalized for covalent biding, such as by click chemistry (e.g., with a nucleotide that is 5-DBCO-PEG4-dUTP or 5-TCO-PEG4-dUTP). Each of the plurality of binding sites can comprise at most one amplification product. Two binding sites of the plurality of binding sites can comprise two different amplification products. The rolling circle amplification can comprise extending an amplification primer (or a splint primer) using a plurality of template nucleic acid as templates to generate the plurality of nucleic acids prior to delivering the plurality of nucleic acids to the plurality of binding sites. The amplification primer (or the splint primer) can be part of a DNA tile.

### Continuous Active Chemistry

DNA clusters are formed or captured and immobilized randomly on a continuously functional surface, which constitutes a major hurdle for sequencing clusters that are very close. The method disclosed herein provides a solution for such key problem by creating pads of active chemistry that is required for either capturing and amplification of a library or covalent immobilization of a DNA cluster. Since the pads are fabricated by a shadow mask of self-assembled polystyrene beads, the average center-to-center distance of the pads as well as average pad diameter can be tuned according to DNA cluster sizes to ensure a minimum distance between DNA features on the surface. DNA clusters can only exist on the designated pads of active chemistry and not on the interstitials in between the pads (FIG. 12).

### Flow Cell Surface

Disclosed herein include embodiments of a flow cell surface. The flow cell surface can be generated using any method disclosed herein. The flow cell surface can be generated using colloidal self-assembly (bottom-up) of particles described herein, as compared to top-down lithography. In some embodiments, the flow cell surface comprises a plurality of binding sites (or reaction sites or active sites) of at least 10,000 binding sites. Each of the plurality of binding sites can be circular, round, oval, rectangular, or square. Other shapes are also contemplated by the disclosure. Each of the plurality of binding sites can have a center point and a diameter. In some instances, the flow cell surface includes a plurality of binding sites of at least 10,000 ordered, well packed, and/or high density binding sites separated by disjunctions (e.g., 5, 10, 20, 30, 40, 50, 60, 70, 80, 90, 100, or more disjunctions) that are not predetermined and/or are randomly distributed (or arranged). Alternatively or additionally, the flow cell surface contains a plurality of binding sites of at least 10,000 ordered, well packed, and/or high density binding sites separated by disjunctions. The configurations of the ordered binding sites and/or the disjunctions can be non-predetermined and/or can be randomly distributed (or arranged). In certain instances, the flow cell surface comprises a plurality of binding sites of at least 10,000 binding sites. The plurality of binding sites can include a first plurality of ordered binding sites and a second plurality of ordered binding sites separated by a disjunction that is not predetermined and/or is randomly distributed or arranged (see FIGS. 4A-4D for examples). A first configuration of the first plurality of ordered binding sties and a second configuration of the second plurality of ordered binding sites can be different (see FIGS. 4A-4D for examples) or the same. The binding sites on a flow cell surface can be ordered, well packed, and/or can have high density. The positions (or locations) of the binding sites on the flow cell surface can be random and non-predetermined. The sizes and location of the disjunctions on the flow cell surface can be random and non-predetermined.

Different embodiments of the present disclosure contemplate different separations or pitches between any two neighbor binding sites. The pitch between any binding site and any nearest neighbor binding site, measured from the center of the first binding site to the center of the nearest neighbor binding site, can be at least twice as large as the diameter of the first binding site (see FIG. 4A for an example). Separation between any binding site and any nearest neighbor binding site, measured from an edge of the first binding site to a center of the nearest neighbor binding site is at least twice as large as the diameter of the first binding site (see FIG. 4A for an example). The two edges are closer than (or at least as close as) the distance between any other edge of the first binding site and any edge of the second binding site.

A desirable pitch (or the separation) can be obtained so the binding sites (or spots) are well separated, which can for example improve binding site identification at ultra-high density. The pitch (or separation) can be, be about, be at least, be at least about, be at most, or be at most about, 1×, 1.1×, 1.2×, 1.3×, 1.4×, 1.5×, 1.6×, 1.7×, 1.8×, 1.9×, 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, 4.9×, 5×, 5.1×, 5.2×, 5.3×, 5.4×, 5.5×, 5.6×, 5.7×, 5.8×, 5.9×, 6×, 6.1×, 6.2×, 6.3×, 6.4×, 6.5×, 6.6×, 6.7×, 6.8×, 6.9×, 7×, 7.1×, 7.2×, 7.3×, 7.4×, 7.5×, 7.6×, 7.7×, 7.8×, 7.9×, 8×, 8.1×, 8.2×, 8.3×, 8.4×, 8.5×, 8.6×, 8.7×, 8.8×, 8.9×, 9×, 9.1×, 9.2×, 9.3×, 9.4×, 9.5×, 9.6×, 9.7×, 9.8×, 9.9×, 10×, of a number or a range between any two of these values, as large as the diameter of the first binding site. For example, the separation is at least three times as large as the diameter of first binding site.

A certain percentages of the binding sites on a flow cell surface can be ordered, well-packed, and/or high density, such as 1%, 2%, 3%, 4%, 5%, 6%, 7%, 8%, 9%, 10%, 20%, 30%, 40%, 50%, 60%, 70%, 80%, 90%, 99%, or more. At least a portion of the plurality of binding sites (such as substantially all or all of the binding sites) can be randomly arrayed on the flow cell surface using any method disclosed herein such as 1%, 2%, 3%, 4%, 5%, 6%, 7%, 8%, 9%, 10%, 20%, 30%, 40%, 50%, 60%, 70%, 80%, 90%, 99%, or more. The plurality of binding sites (or a percentage of the binding sites, such as 1%, 2%, 3%, 4%, 5%, 6%, 7%, 8%, 9%, 10%, 20%, 30%, 40%, 50%, 60%, 70%, 80%, 90%, 99%, or more) may not be arrayed on the flow cell surface at a predetermined set of locations using any method disclosed herein. At least a portion of the plurality of binding sites do not share a common pattern, such as 1%, 2%, 3%, 4%, 5%, 6%, 7%, 8%, 9%, 10%, 20%, 30%, 40%, 50%, 60%, 70%, 80%, 90%, 99%, or more. A pattern can include positions or relative positions of the binding sites within the pattern. A pattern can include one or more configurations of the particles (such as hexagonal configurations, equilateral triangle configurations, straight line configurations, and linear configurations described herein) within the pattern. The plurality of binding sites or a percentage of the binding sites (such as 1%, 2%, 3%, 4%, 5%, 6%, 7%, 8%, 9%, 10%, 20%, 30%, 40%, 50%, 60%, 70%, 80%, 90%, 99%, or more) can comprise unpatterned binding sites. In comparison, a flow cell surface arrayed using top-down lithography include binding sites at predetermined locations having predetermined patterns and areas of binding sites sharing a common pattern.

The flow cell surface can be functionalized by the binding sites thereon as described above. The plurality of binding sites can comprise a plurality of nucleic acids, such as a concatemeric nucleic acid or a nucleic acid tether. One, at least one, or each, of the plurality of binding sites can comprise one, or at most one, of the plurality of nucleic acids. The plurality of nucleic acids can attached to a plurality of beads, such as emPCR beads. One, at least one, or each, of the plurality of binding sites can comprise one, or at most one, of the plurality of beads. The plurality of nucleic acids can comprise a plurality of concatemeric nucleic acids, such as DNA tiles, and amplification products from rolling circle amplification (RCA).

The disclosure contemplates different percentages of the plurality of reaction sites each comprising no more than one nucleic acid (such as nucleic acid tether). The percentage of the plurality of reaction sites each comprising no more than one nucleic acid can be, be about, be at least, be at least about, be at most, or be at most about, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or a number or a range between any two of these values. For example, at least 90% of the reaction sites comprise no more than one nucleic acid.

Clonal amplification can be performed using the flow cell surface to generate clonal populations on or at the binding sites. The percentage of the plurality of reaction sites each comprising a clonal population of no more than one originating nucleic acid can be different in different embodiments. In some embodiments, percentage of the plurality of reaction sites each comprising clonal populations of no more than one originating nucleic acid can be, be about, be at least, be at least about, be at most, or be at most about, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or a number or a range between any two of these values. For example, at least 90% of the reaction sites comprise clonal populations of no more than one originating nucleic acid each.

An originating nucleic acid on a first reaction site of two reaction sites and an originating nucleic acid on a second reaction site of the two reaction sites can be distinct. The number of reaction sites of the plurality of reaction sites with distinct originating nucleic acids can be different in different embodiments. The number of reaction sites of the plurality of reaction sites with distinct originating nucleic acids can be, be about, be at least, be at least about, be at most, or be at most about, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or a number or a range between any two of these values.

The methods of the disclosure can be used to generate flow cell surfaces. A plurality of flow cell surfaces can each comprise at least 10,000 binding sites (or active sites or reaction sites). No two flow cell surfaces of the plurality of flow cell surfaces share a congruent binding site configuration comprising all of the binding sites on each of the plurality of flow cell surfaces. In some embodiments, two flow cell surfaces of said plurality of flow cell surfaces share a congruent binding site configuration comprising less than all of the binding sites on each of the two flow cell surfaces.

A flow cell surface, or every flow cell surface, can comprise a binding site configuration of a number of binding sites that is not congruent (e.g., the same) with the binding site configuration of the same number of binding sites on any other flow cell surface or of the binding sites in the corresponding area on any other flow cell surface. The number of binding sites can be, be about, be at least, be at least about, be at most, or be at most about, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, or a number or a range between any two of these values. For example, a flow cell surface, or every flow cell surface, comprises at least three neighbor binding sites in an equilateral triangle configuration that is not congruent with the equilateral triangle configuration of any three neighbor binding sites on any other flow cell surface. For example, a flow cell surface, or every flow cell surface, comprises at least ten binding sites that is not congruent with any at least ten binding sites on any other flow cell surface.

A flow cell surface, or every flow cell surface, can comprise at least a percentage of the plurality of binding sites on the flow cell surface that is not congruent with such percentage of the plurality of binding sites on any other flow cell surface. The percentage can be, be about, be at least, be at least about, be at most, or be at most about, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, or a number or a range between any two of these values. For example, a flow cell surface, or every flow cell surface, comprises at least 5% of the plurality of binding sites on the flow cell surface that is not congruent with any 5% of the plurality of binding sites on any other flow cell surface. For example, a flow cell surface, or every flow cell surface, comprises at least 10% of the plurality of binding sites on the flow cell that is not congruent with any 10% of the plurality of binding sites on any other flow cell surface.

A first flow cell surface of the plurality of flow cell surfaces can comprise a first binding site array having a first region of regular binding site array configuration and a second region of regular binding site array configuration separated by a first disjunction, such as a first region of irregular binding site array configuration. A second flow cell surface of the plurality of flow cell surfaces can comprise a second binding site array having a first region of regular binding site array configuration and a second region of regular binding site array configuration separated by a second disjunction, such as a second region of irregular binding site array configuration. The first binding site array and the second binding site array can be different. FIGS. 4A-4D each illustrates a different binding site array with two different binding site array configurations. The first binding site array can comprise 5%, 10%, 15%, 20%, 30%, 40%, 50%, 60%, 70%, 80%, 90%, 99.9%, or all of the binding sites on the first flow cell surface. The second binding site array can comprise 5%, 10%, 15%, 20%, 30%, 40%, 50%, 60%, 70%, 80%, 90%, 99.9%, or all of the binding sites on the second flow cell surface. A binding site array configuration can comprise all the binding sites of the binding site array configuration

The first disjunction and the second disjunction can be different. The first region of irregular binding site array configuration and the second region of irregular binding site array configuration may be congruent or may not be congruent. For example, the first region of irregular binding site array configuration can comprise at least three binding sites that are not congruent with any three binding sites of the second region of irregular binding site array configuration. The first region of regular binding site array configuration and the second region of regular binding site array configuration of the first binding site array can comprise binding sites that may be congruent or may not be congruent. The first region of regular binding site array configuration and the second region of regular binding site array configuration of the second binding site array comprise binding sites that may be congruent or may not be congruent. The first region of regular binding site array configuration of the first binding site array and the first region of regular binding site array configuration of the second binding site array may be congruent or may not be congruent. The second region of regular binding site array configuration of the first binding site array and the second region of regular binding site array configuration of the second binding site array may be congruent or may not be congruent.

The number of binding sites in a binding site array, a region of regular binding site array configuration, a disjunction, or a region of irregular binding site array configuration can be non-predetermined. The number of binding sites in a binding site array, a region of regular binding site array configuration, a disjunction, or a region of irregular binding site array configuration can be, be about, be at least, be at least about, be at most, or be at most about, 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, or a number or a range between any two of these values. For example, the first region of regular binding site array configuration comprises at least 500 binding sites. The second region of regular binding site array configuration can comprise at least 500 binding sites. The first region of region of irregular binding site array configuration can comprise at least 50 binding sites. The second region of irregular binding site array configuration can comprise at least 50 binding sites.

The first region of regular binding site array configuration can comprise a percentage of the binding sites on the first flow cell surface and/or of the first binding array (or on the first flow cell surface and/or of the first binding array). The second region of regular binding site array configuration can comprise a percentage of the binding sites on the first flow cell surface and/or of the first binding array (or on the first flow cell surface and/or of the first binding array). The first region of irregular binding site array configuration can comprise a percentage of the binding sites on the first flow cell surface and/or of the first binding array. The second region of irregular binding site array configuration can comprise a percentage of the binding sites on the second flow cell surface and/or of the second binding array. The percentages for any two (or all) the first region of regular binding site array configuration, the second region of regular binding site array configuration, the first region of irregular binding site array configuration, and the second region of irregular binding site array configuration can be identical or different. The percentage can be, be about, be at least, be at least about, be at most, or be at most about, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, or a number or a range between any two of these values. For example, the first region of regular binding site array configuration comprises at least 5% of the binding sites on the first flow cell surface. The second region of regular binding site array configuration can comprise at least 5% of the binding sites on the first flow cell surface. The first region of irregular binding site array configuration can comprise at least 0.5% of the binding sites on the first flow cell surface. The second region of irregular binding site array configuration can comprise at least 0.5% of the binding sites on the second flow cell surface.

The plurality of binding sites on one, one or more, or each, of the plurality of flow cell surfaces can comprise a plurality of nucleic acids, such as concatemeric nucleic acids, nucleic acid tethers, amplification primers (or splint primers), amplified nucleic acids (e.g., target nucleic acids amplified using RCA), and DNA tiles. One, at least one, or each, of the plurality of binding sites on each of the plurality of flow cell surfaces can comprise one, or at most one, of the plurality of nucleic acids comprised in the plurality of binding sites on the flow cell surface. The plurality of nucleic acids comprised in the plurality of binding sites on each of the plurality of flow cell surfaces can be attached to a plurality of beads, such as emPCR beads. One, at least one, or each, of the plurality of binding sites can comprise one, or at most one, of the plurality of beads.

For a flow cell surface, one, one or more, or each of the plurality of nucleic acids on the plurality of binding sites on the flow cell surface comprises an amplification primer (or a splint primer) an amplification product from rolling circle amplification (RCA), a DNA tile, or a combination thereof. One, one or more, or each of the plurality of nucleic acids on the plurality of binding sites on the flow cell surface can comprise a first functional moiety capable of participating in a click chemistry reaction, such as methyltetrazine (MTz). One, one or more, or each of the plurality of binding sites on the flow cell surface comprises a second functional moiety capable of participating in the click chemistry reaction, such as trans-cyclooctene (TCO). The DNA tile can comprise an amplification primer or a splint primer, optionally the amplification primer or the splint primer is attached to the binding site, and optionally the amplification primer or the splint primer is attached to the binding site via the click chemistry reaction involving the first functional moiety and the second functional moiety.

The percentage of the plurality of binding sites on one, one or more, or each, of the plurality of flow cell surfaces comprises at least one nucleic acid of the pluralities of nucleic acids and/or one bead of the pluralities of beads can be different. The percentage of the plurality of binding sites on one, one or more, or each, of the plurality of flow cell surfaces comprises at least one nucleic acid of the pluralities of nucleic acids and/or one bead of the pluralities of beads can be, be about, be at least, be at most, or be at most about, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. For example, at least 50% of the plurality of binding sites on one, one or more, or each, of the plurality of flow cell surfaces comprises at least one nucleic acid of the pluralities of nucleic acids and/or one bead of the pluralities of beads.

In some embodiments, a plurality of flow cell surfaces (or a plurality of flow cells) can comprise binding sites with different configurations. In some embodiments, each flow cell surface (or a surface of a flow cell) comprises at least 10,000 ordered binding sites separated by disjunctions that are at non-predetermined locations and/or are randomly distributed (or arranged). No two flow cell surfaces comprise an identical configuration of the disjunctions on the flow cell surface. In some instances, each flow cell surface (or a surface of a flow cell) comprises at least 10,000 ordered binding sites separated by disjunctions. Configurations (e.g., the relative positions or locations) of the ordered binding sites and the disjunctions are not predetermined and/or are randomly distributed (or arranged). No two flow cell surfaces can comprise an identical configuration of the ordered binding sites and disjunctions. Other embodiments provide each flow cell surface (or a surface of a flow cell) comprising at least 10,000 ordered binding sites separated by irregular regions of binding sites at non-predetermined locations and/or are randomly distributed (or arranged). No two flow cell surfaces can comprise an identical configuration of the irregular regions on the flow cell surface. Alternatively or additionally, each flow cell surface (or a surface of a flow cell) comprises at least 10,000 ordered binding sites separated by irregular regions of binding sites. Configurations of the ordered binding sites and the irregular regions of bindings are not predetermined and/or are randomly distributed (or arranged). No two flow cell surfaces can comprise an identical configuration of the ordered binding sites and the irregular regions of binding sites. In some instances, each flow cell surface (or a surface of a flow cell) comprises at least 10,000 binding sites in regular regions of binding sites and irregular regions of binding sites separating the regular regions of binding sites. The regular regions and the irregular regions are at non-predetermined locations and/or are randomly distributed (or arranged). No two flow cell surfaces can comprise an identical configuration of the regular regions and/or the irregular regions.

### Rolling Circle Amplification

A method of rolling circle amplification (RCA) is contemplated herein in solution (see FIG. 11 for an illustration) or at the binding sites (see FIG. 14 for an illustration). FIG. 11 shows a schematic illustration of rolling circle amplification (RCA) for cluster generation and Structured Colloidal Arrays of Randomly Assembled Beads (SCARAB) deposition. DNA library is first denatured, and then adapter ends are hybridized to a splint oligo, forming a ring. The library ends are ligated together. In some embodiments, remaining linear DNA is removed by exonucleases. Ring DNA is hybridized to an extension primer to initiate extension by a DNA polymerase. After extension, resulting clusters are deposited onto a SCARAB surface.

In some embodiments, after denaturing library (also referred to herein as target nucleic acids) and annealing the denatured library to a splint, target nucleic acids are circularized. Optionally, the splint can be removed. RCA can occur using an amplification primer. RCA can occur in solution. The amplification products can be deposited onto binding sites. Any step of the process described above can occur in a reaction solution. A reaction solution can include target nucleic acids, splint, primer, Tri HCl, NaCl, MgCl, KCl, dNTPs, EDTA, DTT, NAD, ATP, sucrose, betaine, PEG 200, TritonX100, Tween n80, Taq ligase, T4 PNK, ExoI, ExoIII, Phi29, or a combination thereof. A component in a reaction solution can have a concentration of, of about, of at least, of at least about, of at most, or of at most about, 0.1 nM, 0.2 nM, 0.3 nM, 0.4 nM, 0.5 nM, 0.6 nM, 0.7 nM, 0.8 nM, 0.9 nM, 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, or a number or a range between any two of these vales. A component in a reaction solution can have a concentration of, of about, of at least, of at least about, of at most, or of at most about, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, or a number or a range between any two of these vales. A component in a reaction solution can have a concentration of, of about, of at least, of at least about, of at most, or of at most about, 0.01 U/μL, 0.02 U/μL, 0.03 U/μL, 0.04 U/μL, 0.05 U/μL, 0.06 U/μL, 0.07 U/μL, 0.08 U/μL, 0.09 U/μL, 0.1 U/μL, 0.2 U/μL, 0.3 U/μL, 0.4 U/μL, 0.5 U/μL, 0.6 U/μL, 0.7 U/μL, 0.8 U/μL, 0.9 U/μL, 1 U/μL, or a number or a range between any two of these vales. The reaction solution can have a volume of, of about, of at least, of at least about, of at most, or of at most about, 10, microliter (μL), 20 μL, 30 μL, 40 μL, 50 μL, 60 μL, 70 μL, 80 μL, 90 μL, 100 μL, 0.2 milliliter (mL), 0.3 mL, 0.4 mL, 0.5 mL, 0.6 mL, 0.7 mL, 0.8 mL, 0.9 mL, 1 mL, 2 mL, 3 mL, 4 mL, 5 mL, 6 mL, 7 mL, 8 mL, 9 mL, 10 mL or a number or a range between any two of these values.

### Nucleic Acid Tiles

One requirement for on-surface clustering on a structured surface is a high monoclonal occupancy, as defined by the fraction of pads occupied by a single monoclonal cluster. One route to a high monoclonal occupancy is to maximize the fraction of pads that capture and amplify a single library molecule. A surface consisting of structured pads containing a lawn of splint primers would be inherently limited by Poisson statistics, i.e. a maximum expected monoclonal occupancy of ˜37%. If the number of splint primers could be reduced to one per pad, the Poisson limitation should be relaxed. In certain aspects, the monoclonal occupancy may be at least 50%, at least 80%, or at least 90%.

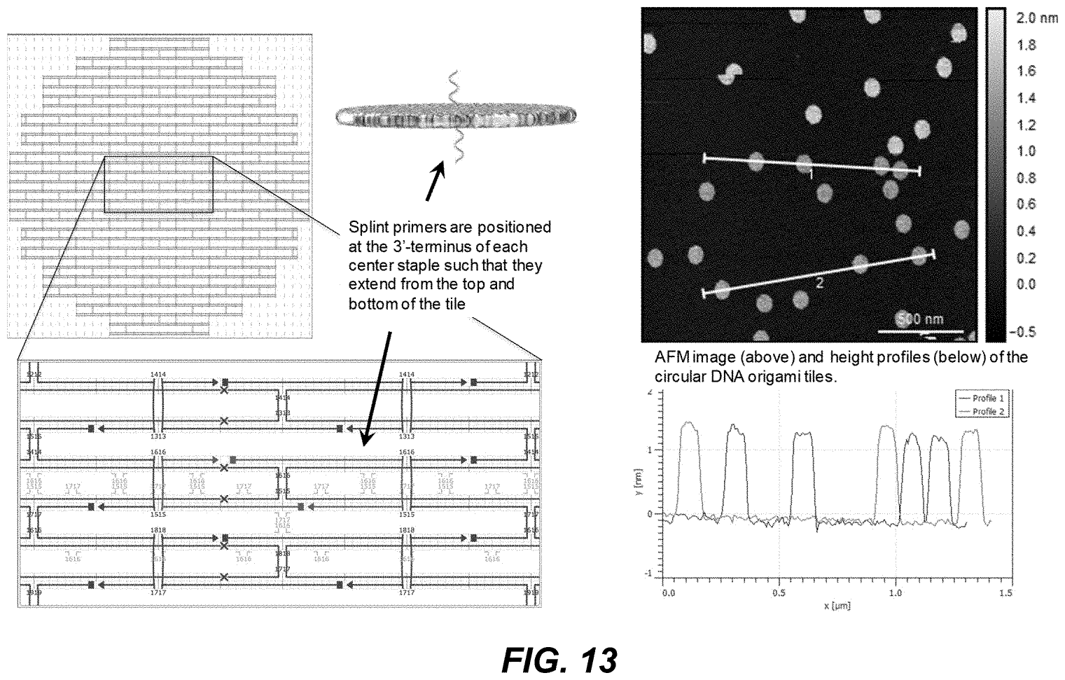

To enable the above approach, disclosed herein is use of a self-assembled circular DNA tile to position a single DNA splint at the center of each pad. As shown in FIG. 13, a DNA tile containing a single splint on each side is captured electrostatically onto each positively charged pad. Single occupancy is achieved though steric exclusion by matching the relative size of the pads and tiles and by optimizing the interstitial surface coating to inhibit adsorption of tiles outside of the pads. The DNA tile then provides the base for the splint, which is used to capture and amplify a single library molecule on each pad. A scaffold may be a genomic DNA, such as genomic DNA of an M13mp18 bacteriophage used in the examples herein. Different DNA origami structures can be found in Rothemund, P. W. (2006). Folding DNA to create nanoscale shapes and patterns. Nature, 440(7082), 297-302; Ke, Y., Lindsay, S., Chang, Y., Liu, Y., & Yan, H. (2008). Self-assembled water-soluble nucleic acid probe tiles for label-free RNA hybridization assays. Science, 319(5860), 180-183; Andersen, E. S., Dong, M., Nielsen, M. M., Jahn, K., Subramani, R., Mamdouh, W., . . . & Kjems, J., et al. (2009). Self-assembly of a nanoscale DNA box with a controllable lid. Nature, 459(7243), 73-76; and Douglas, S. M., Marblestone, A. H., Teerapittayanon, S., Vazquez, A., Church, G. M., & Shih, W. M. (2009). Rapid prototyping of 3D DNA-origami shapes with caDNAno. Nucleic acids research, 37(15), 5001-5006 which are incorporated heirin by reference in their entirety. Examples of alternative scaffolds being used to assemble DNA origami are described in Yang, Y., Han, D., Nangreave, J., Liu, Y., & Yan, H. (2012). DNA origami with double-stranded DNA as a unified scaffold. ACS nano, 6(9), 8209-8215; and Erkelenz, M., Bauer, D. M., Meyer, R., Gatsogiannis, C., Raunser, S., Sacci, B., & Niemeyer, C. M. (2014). A Facile Method for Preparation of Tailored Scaffolds for DNA-Origami. Small, 10(1), 73-77 which are incorporated herein by reference in their entirety.

### DNA Tiles

Circular DNA origami tiles were used to position a single DNA capture primer on each pad. As described elsewhere, the DNA origami method uses a set of short synthetic DNA oligonucleotides, called ‘staples,’ to fold a long (5-50 kb) single-stranded DNA ‘scaffold’ into a predetermined geometry by base-pairing. Each staple contains multiple binding domains, each of which binds to a different region of the scaffold, forming double-crossover motifs. The collective effect of hybridization of the entire set of staples to the scaffold is to constrain the geometry of the scaffold to obey the designed geometry.

Here the tile geometry was designed to approximate the circular shape of the surface pads, and a single capture strand was incorporated into each side of the tile. To accomplish this, the 7249 nt single-stranded genomic DNA from the M13mp18 bacteriophage was used as a scaffold. In the design, the scaffold was rastered back and forth to form a pseudocircle, with the staple strands arranged in a suitable pattern to define the target geometry as shown in Figure X. To position a single capture site at the center of each side of the tile, two of the staples near the center were chosen to serve as the anchors, and the splint sequence was then added to the 3′-terminus of each anchor staple. The location of the 5′ ends of these anchor staples was adjusted to ensure that the two capture strands would protrude from alternate faces of the tile. Short poly-T spacer regions (3-10 nt) could be placed between the staple and capture sequences in order to facilitate efficient capture of the library molecules. Similar short poly-T regions were also incorporated into the staples at the edges of the tile in order to minimize tile aggregation caused by blunt-end stacking.

DNA origami tiles were assembled using a typical protocol as follows. The DNA staple oligos were pooled together in equimolar concentrations into a staple master mix. The staple master mix was then combined with the scaffold DNA in a folding buffer, at final concentrations of 100 nM staple DNA (per sequence), 10 nM scaffold DNA, 40 mM Tris acetate, 12.5 mM magnesium chloride, and 0.1 mM EDTA, in ultrapure water, at a final volume of 50 uL. The folding reaction was then subjected to a thermal annealing protocol. Folded DNA tiles were purified using microcentrifuge purification columns (Amicon Ultra, 100 kDa MWCO).

Purified DNA tiles were deposited onto the surface (see FIG. 14 for an illustration) by incubating the purified tile solution with the surface for times ranging from 0.5-24 hr, then rinsing with a buffer solution containing 40 mM Tris acetate, 12.5 mM magnesium chloride, and 0.1 mM EDTA.

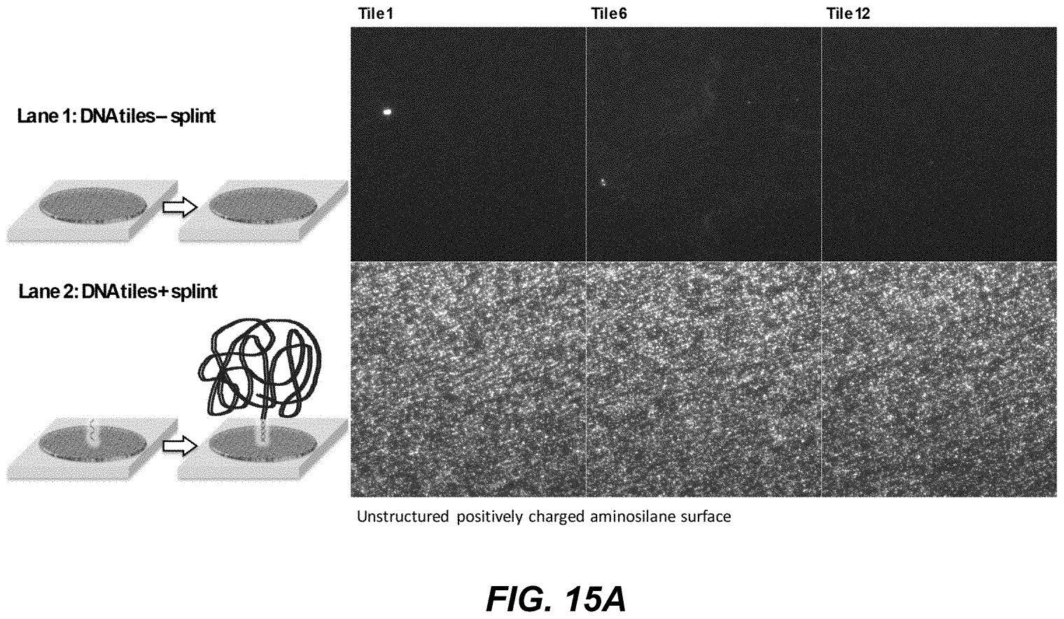

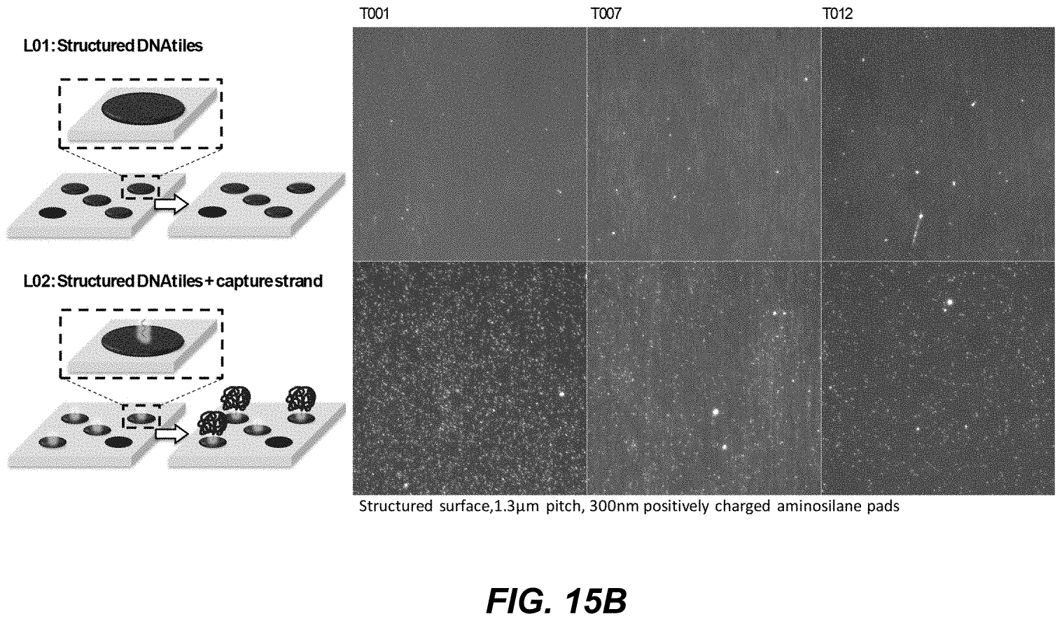

Library molecules were then captured on the DNA tiles by incubating the surface with a solution of DNA library molecules containing tails complementary to the capture strand. Circularization and on-surface amplification of the captured library DNA then proceeded as described elsewhere. FIGS. 15A-15B compare clustering on unstructured DNA tile surface (FIG. 15A) and on structured DNA tile surface (FIG. 15B).

### DNAnemone Tiles

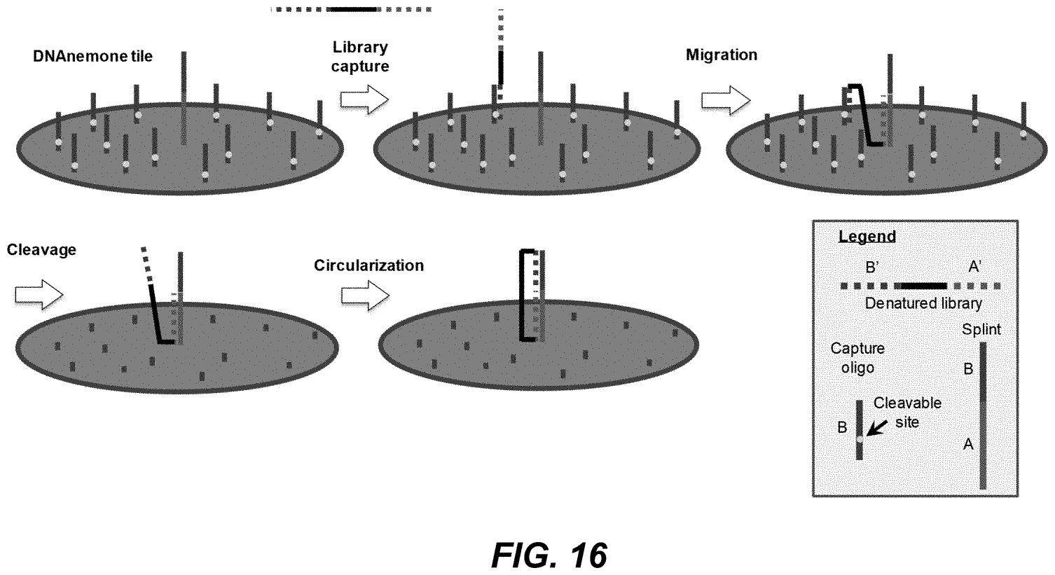

Library capture kinetics are reduced when relying on a single capture oligonucleotide per tile. Capture kinetics may be improved by binding library molecule (e.g., concatemers or circularized nucleic acids) to asymmetric DNA tiles in solution, where one side binds to the library molecule and the other side is functionalized for subsequent binding to a binding site. Examples of asymmetric DNA tiles are described in US patent publication number US20150298090, which is incorporated herein by reference in its entirety. A variation of this strategy utilizing an asymmetric DNA tile can by used to first capture the library molecule in solution, then selectively immobilize the tile onto a substrate. In this approach, the asymmetric tile has a ‘top’ face that captures the library molecule for circularization and amplification, and a ‘bottom’ face that is selectively immobilized onto the substrate after library capture. Carrying out the library capture step in solution prior to substrate immobilization improves the capture kinetics via diffusion but also requires that the tile be immobilized selectively with the top face pointing away from the substrate. To accomplish this, the top face of the tile may displays a splint sequence, which is again concatenated to the 3′-terminus of the central staple in the tile. The opposing bottom face of the tile may display any of a number of surface-anchor groups, which can selectively react with or otherwise bind to complementary groups on the pads of the substrate to immobilize the tile in a specific face-up orientation. For example, the bottom face could display biotin groups to react with a streptavidin-functionalized substrate, amino groups to react with a N-Hydroxysuccinimide-functionalized substrate, thiol groups to react with an alkyne-functionalized substrate, dibenzocyclooctyne (DBCO) groups to react with an azide-functionalized substrate, trans-cyclooctene (TCO) groups to react with a tetrazine-functionalized substrate, or a specific sequence of DNA to be captured by hybridization with the complementary sequence displayed on the substrate. The surface-anchor groups may be concatenated to the ends of specific staples such that they protrude from the bottom face of the tile, with an appropriate spacer of length 1-10 nm consisting of additional nucleotides and/or a polymer spacer such as polyethylene glycol. The asymmetric tiles containing the splint and surface-anchor groups are assembled and purified using an assembly protocol, such as that described herein. The tiles can then be incubated with the library molecules for several tens of minutes at an appropriate temperature ranging from 20-40° C. After the library hybridization step, the tiles can be incubated for several tens of minutes with a structured substrate containing the appropriate chemical functionality on the pads in order to selectively immobilize the tiles onto the pads. After incubation any unbound tiles are washed away using an excess volume of buffer. Another variation of the DNA origami tile includes a single splint sequence, as described above, surrounded by many additional capture oligos capable of hybridizing to a denatured library molecule (see FIG. 16 for an illustration; referred to herein as DNAnemone). By increasing the number of capture oligonucleotides on the DNA tile, the library capture kinetics will be increased even when the DNA tile is immobilized on a surface (e.g., bound to a binding site prior to deposition of a library molecule).

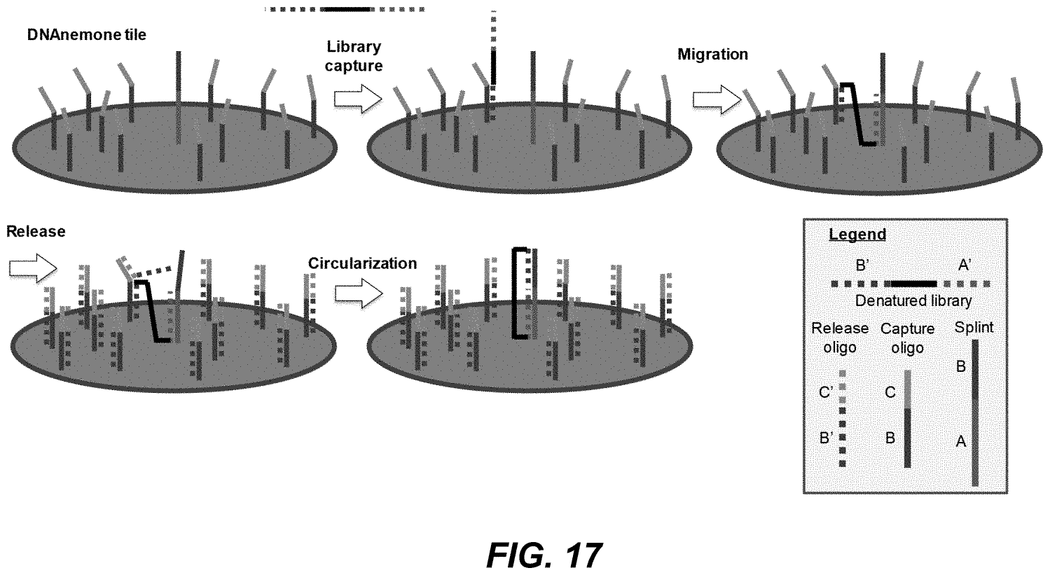

The splint oligonucleotide contains sequence A and sequence B, while the additional capture oligonucleotides contain only sequence B. The melting Temperature™ of sequence A is greater than the Tm of sequence B. Variations of the addition capture oligos include the use of a cleavable site in the sequence (see FIG. 16 for an illustration) and adding an extra sequence C that allows for toehold-mediated strand displacement by an additional release oligonucleotide that is complementary to sequence B and sequence C (see FIG. 17 for an illustration). After library molecules are allowed to hybridize to the tiles, there will be multiple populations of tiles in which one library molecule might be hybridized to some combination of sequence A on the splint, sequence B on the splint, and sequence B on the additional capture oligonucleotides. Depending on the variation of capture oligonucleotide used, the capture oligonucleotides can then be subjected to either cleavage (using a cleavable base in the sequence), or toehold mediated strand displacement (by addition of the release oligo). This cleavage or strand displacement, combined with increasing the temperature above the Tm of sequence B but below that of sequence A, allows for the release of library molecules hybridized to the capture oligonucleotides. Library molecules hybridized to sequence A will stay hybridized. A fluid exchange can be used to eliminate cleavage products and unhybridized library molecules. Decreasing the temperature to below the Tm of sequence B will allow the remaining library molecule hybridized to sequence A to also hybridize to sequence B on the splint. The library molecule can then undergo the clustering process as described elsewhere.

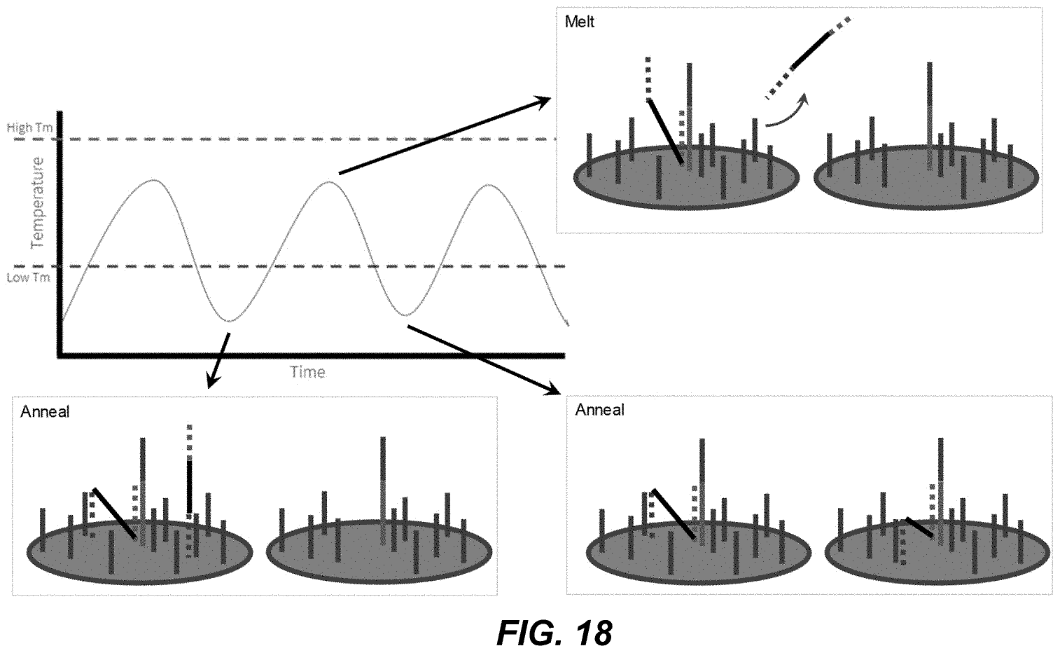

One variation of the DNAnemone hybridization process involves temperature cycling (see FIG. 18 for an illustration). After the initial library molecule hybridization, the temperature can be repeatedly cycled between a temperature that is above the Tm of sequence B but below the Tm of sequence A, and a temperature that is below the Tm of sequence B. This temperature cycling allows library molecules that are hybridized only to sequence B of the additional capture oligonucleotide to dehybridize and populate other tiles.

Also provided herein include a nucleic acid tile comprising a scaffold nucleic acid and a plurality of staple oligonucleotides (see FIG. 13 for an illustration). One, one or more, or each of the plurality of staple oligonucleotides can comprise two binding domains hybridized to different regions of the scaffold nucleic acid, thereby forming a double-crossover motif comprising the scaffold nucleic acid and the staple oligonucleotide. The number of staple oligonucleotides can be, be about, be at least, be at least about, be at most, or be at most about, 10, 20, 30, 40, 50, 60, 70, 80, 90, 100, 110, 120, 130, 140, 150, 160, 170, 180, 190, 200, 250, 300, 350, 400, 450, 500, or a number or a range between any two of these values. The plurality of staple oligonucleotides can comprise a first anchor oligonucleotide that protrudes from a first face of the nucleic acid tile (see FIG. 13 for an illustration). The nucleic acid tile can comprise a deoxyribonucleic acid (DNA). The scaffold nucleic acid can comprise a DNA. The plurality of staple oligonucleotides can comprise DNAs.

In some embodiments, the scaffold nucleic acid forms a pseudocircle by the hybridization of the plurality of staple oligonucleotides. The nucleic acid tile can be pseudocircular in shape. The size (e.g., a width, radius, or diameter) of the nucleic acid tile can be, be about, be at least, be at least about, be at most, or be at most about, 10 nanometer (nm), 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm, 1000 nm, 2 micrometer (μm), 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, or a number or a range between any two of these values. For example, the pitch of two, or any two, adjacent binding sites of the plurality of binding sites is about 1 nm to about 100 μm. The scaffold nucleic acid can be, be about, be at least, be at least about, be at most, be at most about, 1 kilobases (kb), 2 kb, 3 kb, 4 kb, 5 kb, 6 kb, 7 kb, 8 kb, 9 kb, 10 kb, 15 kb, 20 kb, 25 kb, 30 kb, 35 kb, 40 kb, 45 kb, 50 kb, 60 kb, 70 kb, 80 kb, 90 kb, 100 kb, or a number or a range between any two of these values, in length. The scaffold nucleic acid can comprise genomic DNA, such as genomic DNA of M13mp18 bacteriophage. The plurality of staple oligonucleotides can comprise a second anchor oligonucleotide that protrude from a second face of the nucleic acid tile. The first anchor oligonucleotide and the second anchor oligonucleotide can protrude from opposite faces of the nucleic acid tile (see FIG. 13 for an illustration).

An anchor oligonucleotide can comprise a splint sequence (see FIG. 16 for an illustration). The splint sequence can be at the 3′ end of the anchor oligonucleotide. The anchor oligonucleotide can comprise a spacer sequence. The spacer sequence can be 5′ to the splint sequence. The spacer sequence can comprise a poly-T sequence. The spacer sequence can be, be about, be at least, be at most, or be at most about, 2, 3, 4, 5, 6, 7, 8, 9, 10, 15, 20, 30, 40, 50, or a number or a range between any two of these values, nucleotides in length. The splint sequence can comprise a first splint sequence and a second splint sequence (e.g., sequence A and sequence B respectively in FIG. 16). The melting temperature (Tm) of the first splint sequence can be higher than Tm of the second splint sequence. The plurality of staple oligonucleotides can comprise a plurality of capture oligonucleotides each comprising a first capture sequence. The first capture sequence can comprise the second splint sequence (e.g., sequence B in FIG. 16). The capture oligonucleotide may not comprise the first splint sequence. Capture oligonucleotides of the plurality of capture oligonucleotides can be identical. One, one or more, or each of the plurality of capture oligonucleotides can comprise a cleavable site (see FIG. 16 for an illustration), such as a cleavable nucleotide. The capture oligonucleotides can be used to improving library capture kinetics (see FIG. 16 for an illustration; referred to herein as DNAnemone). In some embodiments, one, one or more, or each of the plurality of capture oligonucleotides can comprise a second capture sequence (e.g., sequence C in FIG. 17).

Disclosed herein include methods of forming a nucleic acid tile. In some embodiments, a method of forming a nucleic acid tile comprises providing a staple solution comprising the plurality of staple oligonucleotides. The method can comprise providing a scaffold solution comprising a scaffold nucleic acid. The method can comprise combining the staple solution and the scaffold solution to form a reaction solution. The method can comprise subject the reaction solution to thermal annealing, thereby forming the nucleic acid tile. The reaction solution can differ. A component in the reaction solution can have a concentration of, of about, of at least, of at least about, of at most, or of at most about, 0.1 nM, 0.2 nM, 0.3 nM, 0.4 nM, 0.5 nM, 0.6 nM, 0.7 nM, 0.8 nM, 0.9 nM, 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, or a number or a range between any two of these vales. For example, the reaction solution comprises each of the plurality of staple oligonucleotides at about 100 nanomolar (nM), the scaffold nucleic acid at about 10 millimolar (mM), tris acetate at about 40 mM, magnesium chloride at about 12.5 mM, and/or EDTA at about 0.1 mM. A component in a reaction solution can have a concentration of, of about, of at least, of at least about, of at most, or of at most about, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, or a number or a range between any two of these vales. The reaction solution can have a volume of, of about, of at least, of at least about, of at most, or of at most about, 10, microliter (μL), 20 μL, 30 μL, 40 μL, 50 μL, 60 μL, 70 μL, 80 μL, 90 μL, 100 μL, 0.2 milliliter (mL), 0.3 mL, 0.4 mL, 0.5 mL, 0.6 mL, 0.7 mL, 0.8 mL, 0.9 mL, 1 mL, 2 mL, 3 mL, 4 mL, 5 mL, 6 mL, 7 mL, 8 mL, 9 mL, 10 mL or a number or a range between any two of these values. The reaction solution can have a volume of, of about, of at least, of at least about, of at most, or of at most about, 10, microliter (μL), 20 μL, 30 μL, 40 μL, 50 μL, 60 μL, 70 μL, 80 μL, 90 μL, 100 μL, 0.2 milliliter (mL), 0.3 mL, 0.4 mL, 0.5 mL, 0.6 mL, 0.7 mL, 0.8 mL, 0.9 mL, 1 mL, 2 mL, 3 mL, 4 mL, 5 mL, 6 mL, 7 mL, 8 mL, 9 mL 10 mL, 20 mL, 30 mL, 40 mL, 50 mL, 100 mL, 1000 mL, 2 liter (L), 3 L, 4 L, 5 L, 6 L, 7 L, 8 L, 9 L, 10 L, or a number or a range between any two of these values. The method can comprise purifying the nucleic acid tile from molecules of the scaffold nucleic acid and molecules of the plurality of staple oligonucleotides that are not parts of molecules of the nucleic acid tile. The method can comprise purifying the nucleic acid tile comprises size-based purification.

A method of rolling circle amplification (RCA) is contemplated herein in solution or at the binding sites. A method of RCA can comprise providing a nucleic acid tile. The method can comprise providing a target nucleic acid. The method can comprise circularizing the target nucleic acid using the splint sequence of the nucleic acid tile. The method can comprise performing rolling circle amplification by extending the splint sequence using the target nucleic acid as the template to generate the nucleic acid tile each comprising concatemeric copies of the target nucleic acid (see FIG. 14 for an illustration).

A method of RCA can comprise providing a plurality of nucleic acid tiles. The method can comprise providing a plurality of target nucleic acids. The method can comprise hybridizing each of the plurality of target nucleic acids to the splint sequence of a nucleic acid tile of the plurality of nucleic acid tiles. The method can comprise circularizing the target nucleic acid hybridized to the splint sequence of the nucleic acid tile. The method can comprise performing rolling circle amplification by extending the splint sequence of the nucleic acid tile using the target nucleic acid hybridized thereto as a template to generate a nucleic acid cluster comprising the nucleic acid tile with the splint sequence thereof extended to include concatemeric copies of the target nucleic acid. FIGS. 15A-15B compare clustering on unstructured DNA tile surface (FIG. 15A) and on structured DNA tile surface (FIG. 15B).

The method can further comprise depositing the nucleic acid tiles onto binding sites of a flow cell surface. The nucleic acids tiles can be deposited onto the binding sites by the attraction between the positively charged binding sites and the negatively charged nucleic acid tile. Alternatively, binding sites may be functionalized with a biomolecule that binds (e.g., by affinity or hybridization) to a concatemer, or to nucleic acid tiles (e.g., that in turn bind to a concatemer). The method can further comprise depositing the nucleic acid tiles onto binding sites of a flow cell surface before RCA (see FIG. 14 for an illustration). The nucleic acids tiles can be deposited onto the binding sites by the attraction between the positively charged binding sites and the negatively charged nucleic acid tile. The method can further comprise depositing the nucleic acid clusters onto binding sites of a flow cell surface after RCA. The percentage of the plurality of binding sites each comprising at most one nucleic acid tile (or at most one nucleic acid cluster) can be, be about, be at least be at least about, be at most, or be at most about, 1%, 2%, 3%, 4%, 5%, 6%, 7%, 8%, 9% 10%, 15%, 20%, 25%, 30%, 35%, 40%, 45%, 50%, 60%, 70%, 80%, 90%, 99%, or a number or a range between any two of these values.

In some instances, the method further comprises removing the plurality of capture oligonucleotides by cleaving the cleavable sites (see FIG. 16 for illustration). In some instances, a target nucleic acid of the plurality of target nucleic acids is hybridized to the first splint sequence of the anchor oligonucleotide and the first capture sequence (or the second splint sequence) of a capture oligonucleotide of the plurality of capture oligonucleotides (see FIG. 17 for an illustration). The method can comprise hybridizing a release oligonucleotide to the capture oligonucleotide. As a result, the target nucleic acid can hybridize to the first splint sequence and the second splint sequence of the anchor oligonucleotide.

In some embodiments, a first target nucleic acid of the plurality of target nucleic acids is hybridized to the first splint sequence of the anchor oligonucleotide and the first capture sequence (or the second splint sequence) of a first capture oligonucleotide of the plurality of capture oligonucleotides (see FIG. 18 for an illustration). A second target nucleic acid of the plurality of target nucleic acids can be hybridized to the second splint sequence of a second capture oligonucleotide of the plurality of capture oligonucleotides. The method can comprise subjecting the nucleic acid tile to thermal cycling. As a result, the second target nucleic acid is released from the second capture oligonucleotide. And the first target nucleic acid is hybridized to the first splint sequence and the second splint sequence of the anchor oligonucleotide. In some embodiments, a first target nucleic acid is hybridized to the first splint sequence and the second splint sequence of the anchor oligonucleotide. A second target nucleic acid of the plurality of target nucleic acids can be hybridized to the second splint sequence of a second capture oligonucleotide of the plurality of capture oligonucleotides. The method can further comprise subjecting the nucleic acid tile to thermal cycling. As a result, the second target nucleic acid is released from the second capture oligonucleotide.

### Aligning Flow Cell Images

Disclosed herein include methods of aligning a plurality of flow cell images. In some embodiments, a method of aligning a plurality of flow cell images is under control of a processor and comprises: obtaining a plurality of flow cell images from a flow cell surface having a first regular or irregular binding site region and a second regular or irregular binding site region separated by an irregular or random binding site region. The method can comprise: aligning the irregular or random binding site region in the plurality of flow cell images to align the plurality of flow cell images. In some embodiments, a method of aligning a plurality of flow cell images is under control of a processor and comprises: obtaining a plurality of flow cell images from a flow cell surface having regular or irregular binding site regions separated by irregular or random binding site regions. The method can comprise: aligning the irregular or random binding site regions in the plurality of flow cell images to align the plurality of flow cell images. In some embodiments, a method of aligning a plurality of flow cell images is under control of a processor and comprises: obtaining a plurality of flow cell images from a flow cell surface comprising ordered binding site separated by disjunctions. The method can comprise: aligning the disjunctions in the plurality of flow cell images to align the plurality of flow cell images. The aligning can comprise translating one flow cell image relative to a second flow cell image. The aligning can comprise rotating one image relative to a second image. The irregularity of the structured surfaces described herein can be used to align flow cell images and/or to identify individual clusters across sequencing cycles.

Different types of flow cell images and different numbers of flow cell images are consistent with the present disclosure. The plurality of flow cell images can comprise florescence emission signals emitted from binding sites of the first regular or irregular binding site region, the second regular or irregular binding site region and the irregular or random binding site region. The plurality of flow cell images can comprise, comprise about, comprise at least, comprise at least about, comprise at most, or comprise at most about, 5, 10, 15, 20, 25, 30, 40, 50, 60, 70, 80, 90, 100, 110, 120, 130, 140, 150, 160, 170, 180, 190, 200, 250, 300, 350, 400, 450, 500, or a number or a range between any two of these values For example, the plurality of flow cell images comprises at least 20 flow cell images. For example, the plurality of flow cell images comprises at least 200 flow cell images.

### Orienting Flow Cell Images

Disclosed herein include methods of orienting a plurality of flow cell images. In some embodiments, a method of orienting a plurality of flow cell images is under control of a processor and comprises: identifying an irregular or random colony region of a plurality of colonies common to a plurality of the flow cell images. The flow cell images can be collected from one or more flow cell surfaces of the present disclosure. An irregular or random colony region can correspond to a disjunction or a region of irregular or random binding site array 406d illustrated in FIGS. 4C-4D. The method can comprise: orienting one or more of the plurality of flow cell images such that the irregular or random colony region is aligned among the plurality of flow cell images. The plurality of colonies on the flow cell can be distributed such that no two colonies are closer than a minimum distance from one another, such as the pitch or separation between the binding sites the where the two colonies are generated. The colonies can be generated at or on binding sites on the flow cell surfaces by amplifying nucleic acids thereon.

The plurality of flow cell images can be collected from a single flow cell. The single flow cell can comprise at least one region of regularly or irregularly arrayed colonies (generated on or at a region of regularly or irregularly arrayed binding sites, such as the regions of regular binding site arrays 402r1 illustrated in FIGS. 4A-4D) and at least one region of irregularly or randomly arrayed colonies (generated on or at a region of irregularly or randomly arrayed binding sites, such as a disjunction or a region of random binding site array 406d illustrated in FIGS. 4C-4D). A flow cell image of the plurality of flow cell images lacking the irregular or random colony region can be discarded from the plurality of flow cell images. The plurality of flow cell images can be collected from a plurality of flow cells. The plurality of flow cells can comprise flow cells each having at least one region of regularly or irregularly arrayed colonies (generated on or at a region of regularly or irregularly arrayed binding sites) and at least one region of irregularly or randomly arrayed colonies (generated on or at a region of irregularly or randomly arrayed binding sites). No two flow cells can share an identical array of irregularly or randomly arrayed colonies. The plurality of images can be sorted such that images having a common region of irregularly or randomly arrayed colonies are grouped together. The plurality of images can be sorted such that images lacking a common region of irregularly or randomly arrayed colonies are differentially grouped.

### Colonies

The pitch (or the separation) of two, or any two, adjacent colonies (or binding sites, reaction sites, or active sites that colonies are at or on) of the plurality of colonies can be different in different embodiments. The pitch of two adjacent colonies (or binding sites, reaction sites, or active sites that colonies are at or on) can be the distance between the centers of the two adjacent sites. The separation of two adjacent colonies (or binding sites, reaction sites, or active sites that colonies are at or on) can be the distance between the closest edges between the two adjacent colonies (or binding sites, reaction sites, or active sites that colonies are in). The pitch (or separation) of two, or any two, adjacent colonies (or binding sites, reaction sites, or active sites that colonies are in) of the plurality of colonies can be, be about, be at least, be at least about, be at most, or be at most about, 10 nanometer (nm), 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm, 1000 nm, 2 micrometer (μm), 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, or a number or a range between any two of these values. For example, the pitch of two, or any two, adjacent colonies of the plurality of colonies is about 1 nm to about 100 μm.

The size (e.g., radius, diameter, width, or height) of one, or one or more, or each, of the plurality of colonies (or binding sites, reaction sites, or active sites that colonies are at or on) can vary. The size of one, or one or more, or each, of the plurality of colonies (or binding sites, reaction sites, or active sites that colonies are at or on) can be, be about, be at least, be at least about, be at most, or be at most about, 10 nanometer (nm), 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm, 1000 nm, 2 micrometer (μm), 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, or a number or a range between any two of these values. For example, the size of one, or one or more, or each, of the plurality of colonies is about 1 nm to about 100 μm.

The number of colonies in the plurality of colonies can depend on or be similar to the number of binding sites, reaction sites, or active sites that colonies are at or on. The number of colonies in the plurality of colonies (or binding sites, reaction sites, or active sites that colonies are at or on) can be, be about, be at least, be at least about, be at most, or be at most about, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, 10,000,000, 20,000,000, 30,000,000, 40,000,000, 50,000,000, 60,000,000, 70,000,000, 80,000,000, 90,000,000, 100,000,000, 200,000,000, 300,000,000, 400,000,000, 500,000,000, 600,000,000, 700,000,000, 800,000,000, 900,000,000, 1,000,000,000, or a number or a range between any two of these values. For example, the number of colonies in the plurality of colonies is about 10,000 colonies to about 10,000,000 colonies.

### Sorting Flow Cell Images

Also provided are methods of sorting a plurality of flow cell images. In some embodiments, a method of sorting a plurality of flow cell images can be under control of a processor and comprise: obtaining a plurality of flow cell images. The method can comprise: identifying a first regular or irregular binding site region and a second regular or irregular binding site region separated by an irregular or random binding site region (or disjunction) in each of the plurality of cell images. The flow cell images can be collected from one or more flow cell surfaces of the present disclosure. The first and regular or irregular binding site regions in a flow cell image can correspond to the regular binding site arrays 402r1, 402r2 illustrated in FIGS. 4A-4D. The irregular or irregular binding site region in a flow cell image can correspond to a disjunction or a region of random binding site array 406d illustrated in FIGS. 4C-4D). The method can comprise: sorting the plurality of flow cell images such that flow cell images having an identical irregular or random binding site region are assigned to a common group and two flow cell images having different irregular or random binding site region are assigned to different common groups.

Alternatively or additionally, a method of sorting a plurality of flow cell images is under control of a processor and comprises: obtaining a plurality of flow cell images. The flow cell images can be collected from one or more flow cell surfaces of the present disclosure. The method can comprise: identifying an irregular or random binding site region in each of the plurality of flow cell images that separates a first regular or irregular binding site region and a second regular or irregular binding site region in the flow cell image. The method can comprise: sorting the plurality of flow cell images such that flow cell images having an identical irregular or random binding site region are assigned to a common group and two flow cell images having different irregular or random binding site region are assigned to different common groups. The method can comprise one or more additional steps disclosed herein. For example, the method can comprise orienting flow cell images of the plurality of flow cell images in each common group such that first binding sites in the flow cell images in the each common group are aligned and the second binding sites in the flow cell image images in the each common group are aligned. Flow cell images having different irregular or random binding site regions can be assigned to different groups.

### Performing Quality Assessment

The present disclosure provides methods of performing quality assessment on an image. In some embodiments, a method of performing quality assessment is under control of a processor and comprises: receiving an image collected from a surface (such as a flow cell surface) comprising a plurality of binding sites. The binding sites can comprise colonies. The method can comprise: identifying a first signal from a first binding site (or the colony thereon). The method can comprise: identifying a second signal from a second binding site (or the colony thereon). The method can comprise: determining a distance separating the first binding site from the second binding site. The method can comprise: negatively assessing the image if the distance is below a threshold. The threshold can be the expected distance between two neighbor binding sites on the surface.

Negatively assessing the image can be accomplished through various approaches. Non-limiting examples of negatively assessing the image include discarding the image; discarding the first signal and the second signal from the image; discarding any signal from the first binding site and any signal from the second binding site; tagging at least one of the first binding site and the second binding site as out of focus; and refocusing at least one of the first binding site and the second binding site. The refocusing can vary. Non-limiting examples of refocusing include reducing a size of the first signal; reducing a size of the first signal and a size of the second signal; increasing an intensity of the first signal; increasing an intensity of the first signal and an intensity of the second signal; and reducing a size of the first signal and a size the second signal and increasing an intensity of the first signal and an intensity of the second signal.

Another example of negatively assessing the image includes determining an out-of-focus value based on the distance and the threshold. A further example of negatively assessing the image includes determining an out-of-focus value based on the distance and an ideal distance. The out-of-focus value can be a ratio of the distance and the threshold, a ratio of the distance and the ideal distance threshold, or a combination thereof. The out-of-focus value can be a difference of the distance and the threshold, a difference of the distance and the ideal distance threshold, or a combination thereof. Negatively assessing the image can include: causing a refocused image to be collected from the surface based on the out-of-focus value; and/or receiving a refocused image collected from the surface based on the out-of-focus value. Negatively assessing the image can include: adjusting a focus of an imaging system that collected the image based on the out-of-focus value; and/or causing the imaging system to adjust a focus of the imaging system based on the out-of-focus value. Negatively assessing the image can include: causing a refocused image to be collected from the surface. Negatively assessing the image can comprise receiving a refocused image to collected from the surface.

Determining the distance can be accomplished through various approaches. For example, determining the distance comprises measuring a distance from the first binding site to the second binding site using a central point of the first binding site and a central point of the second binding site. As another example, determining the distance comprises measuring a distance from the first binding site to the second binding site using an edge of the first binding site and an edge of the second binding site.

Various binding site configurations that are random and/or not predetermined are consistent with the disclosure. For example, at least a portion of said plurality of binding sites is randomly arrayed on said flow cell surface. As an example, at least a portion of said plurality of binding sites is not arrayed on said flow cell surface in a predetermined set of locations. As another example, at least a portion of said plurality of binding sites does not share a common pattern. As a further example, at least said plurality of binding sites comprises unpatterned binding sites. For example, said plurality of binding sites is randomly arrayed. As one example, said plurality of binding sites is arrayed so as to form a first region having regularly or irregularly positioned (or ordered) binding sites and a second region having randomly positioned binding sites.

### Flow Cell Imaging System

Disclosed herein include flow cell imaging systems. A flow cell imaging system comprises: a flow cell having distributed thereon a plurality of binding sites, at least some of the binding sites are randomly distributed. The flow cell can have a flow cell surface generated using any method of the disclosure. The flow cell imaging system can comprise: an excitation source to excite fluorophores at the binding sites. The flow cell imaging system can comprise: an image digitization interface comprising a plurality of pixels. The plurality of binding sites can be distributed such that no two binding sites generate emission signals that are assigned to a common pixel. Alternatively or additionally, at least some of the binding sites are regularly or irregularly distributed. The plurality of binding sites can be present at various densities, such as a density of about 200 k/mm² to about 2,000 k/mm², or at a density of at least 600 k/mm², at least 800 k/mm², at least 1,000 k/mm², or more The number of binding sites can vary, such as at least 100,000 binding sites, at least 1,000,000 binding sites, at least 10,00,000 binding sites, or more.

### Sequencing-by-Binding

“Sequencing-by-binding” refers to a sequencing technique wherein specific binding of a polymerase and cognate nucleotide to a primed template nucleic acid is used for identifying the next correct nucleotide to be incorporated into the primer strand of the primed template nucleic acid. The specific binding interaction need not result in chemical incorporation of the nucleotide into the primer. The specific binding interaction can precede chemical incorporation of the nucleotide into the primer strand or precedes chemical incorporation of an analogous, next correct nucleotide into the primer. Thus, identification of the next correct nucleotide can take place without incorporation of the next correct nucleotide.

Sequencing-by-binding (SBB) has been described in U.S. Pat. Nos. 10,443,098, 10,246,744, and U.S. Patent Application Publication No. 2018,0044727; the content of each of which is incorporated herein by reference in its entirety. Briefly, in SBB the polymerase undergoes conformational transitions between open and closed conformations during discrete steps of the reaction. In one step, the polymerase binds to a primed template nucleic acid to form a binary complex, also referred to herein as the pre-insertion conformation. In a subsequent step, an incoming nucleotide is bound and the polymerase fingers close, forming a pre-chemistry conformation comprising the polymerase, primed template nucleic acid and nucleotide; wherein the bound nucleotide has not been incorporated. This step, also referred to herein as an examination step, may be followed by a chemical incorporation step wherein a phosphodiester bond is formed with concomitant pyrophosphate cleavage from the nucleotide (nucleotide incorporation). The polymerase, primed template nucleic acid and newly incorporated nucleotide produce a post-chemistry, pre-translation conformation. As both the pre-chemistry conformation and the pre-translocation conformation comprise a polymerase, primed template nucleic acid and nucleotide, wherein the polymerase is in a closed state, either conformation may be referred to herein as a ternary complex. The polymerase configuration and/or interaction with a nucleic acid may be monitored during an examination step to identify the next correct base in the nucleic acid sequence. Before or after incorporation, reaction conditions can be changed to disengage the polymerase from the primed template nucleic acid, and changed again to remove from the local environment any reagents that inhibit polymerase binding.

Generally speaking, the SBB procedure includes an “examination” step that identifies the next template base, and optionally an “incorporation” step that adds one or more complementary nucleotides to the 3′-end of the primer component of the primed template nucleic acid. Identity of the next correct nucleotide to be added is determined either without, or before chemical linkage of that nucleotide to the 3′-end of the primer through a covalent bond. The examination step can involve providing a primed template nucleic acid to be used in the procedure, and contacting the primed template nucleic acid with a polymerase enzyme (e.g., a DNA polymerase) and one or more test nucleotides being investigated as the possible next correct nucleotide. Further, there is a step that involves monitoring or measuring the interaction between the polymerase and the primed template nucleic acid in the presence of the test nucleotides.

An examination step typically includes the following substeps: (1) providing a primed template nucleic acid (i.e., a template nucleic acid molecule hybridized with a primer that optionally may be blocked from extension at its 3′-end); (2) contacting the primed template nucleic acid with a reaction mixture that includes a polymerase and at least one nucleotide; (3) monitoring the interaction of the polymerase with the primed template nucleic acid molecule in the presence of the nucleotide(s) and without chemical incorporation of any nucleotide into the primed template nucleic acid; and (4) determining from the monitored interaction the identity of the next base in the template nucleic acid (i.e., the next correct nucleotide).

The examination step may be controlled so that nucleotide incorporation is either attenuated or accomplished. If nucleotide incorporation is attenuated, a separate incorporation step may be performed. The separate incorporation step may be accomplished without the need for monitoring, as the base has already been identified during the examination step. If nucleotide incorporation proceeds during examination, subsequent nucleotide incorporation may be attenuated by a stabilizer that traps the polymerase on the nucleic acid after incorporation. A reversibly terminated nucleotide may be used in the incorporation step to prevent the addition of more than one nucleotide during a single cycle.

The sequencing-by-binding method allows for controlled determination of a template nucleic acid base without the need for labeled nucleotides, as the interaction between the polymerase and template nucleic acid can be monitored without a label on the nucleotide. The controlled nucleotide incorporation can also provide accurate sequence information of repetitive and homopolymeric regions without necessitating use of a labeled nucleotide. Moreover, template nucleic acid molecules may be sequenced under examination conditions which do not require attachment of template nucleic acid or polymerase to a solid-phase support. However, in certain preferred embodiments, primed template nucleic acids to be sequenced are attached to a solid support, such as an interior surface of a flow cell.

The examination step may be controlled, in part, by providing reaction conditions to prevent chemical incorporation of a nucleotide, while allowing determination of the identity of the next correct base on the primed template nucleic acid molecule. Such reaction conditions may be referred to as examination reaction conditions.

Examination typically involves detecting polymerase interaction with a template nucleic acid. Detection may include optical, electrical, thermal, acoustic, chemical and mechanical means. Generally, the examination step involves binding a polymerase to the polymerization initiation site of a primed template nucleic acid in a reaction mixture comprising one or more nucleotides, and monitoring the interaction. The examination step of the sequencing reaction may be repeated 1, 2, 3, 4 or more times prior to the incorporation step. The examination and incorporation steps may be repeated until the desired sequence of the template nucleic acid is obtained.

SBB involves contacting of the primed template nucleic acid molecule with a reaction mixture that includes a polymerase and one or more nucleotide molecules preferably occurs under conditions that stabilize formation of the ternary complex, and that destabilize formation of binary complexes. The formation of the ternary complex or the stabilized ternary complex can be employed to ensure that only one nucleotide is added to the template nucleic acid primer per cycle of sequencing, wherein the added nucleotide is sequestered within the ternary complex. The controlled incorporation of a single nucleotide per sequencing cycle enhances sequencing accuracy for nucleic acid regions comprising homopolymer repeats.

In SBS, a reaction mixture used in the examination step can include 1, 2, 3, or 4 types of nucleotide molecules. The nucleotides can be selected from dATP, dTTP (or dUTP), dCTP, and dGTP. The reaction mixture can comprise one or more triphosphate nucleotides and one or more diphosphate nucleotides. A ternary complex can form between the primed template nucleic acid, the polymerase, and any one of the four nucleotide molecules so that four types of ternary complexes may be formed.

Monitoring or measuring the interaction of the polymerase with the primed template nucleic acid molecule in the presence of a nucleotide molecule may be accomplished in many different ways. For example, monitoring can include measuring association kinetics for the interaction between the primed template nucleic acid, the polymerase, and any one of the four nucleotide molecules. Monitoring the interaction of the polymerase with the primed template nucleic acid molecule in the presence of a nucleotide molecule can include measuring equilibrium binding constants between the polymerase and primed template nucleic acid molecule (i.e., equilibrium binding constants of polymerase to the template nucleic acid in the presence of any one or the four nucleotides). Thus, for example, the monitoring includes measuring the equilibrium binding constant of the polymerase to the primed template nucleic acid in the presence of any one of the four nucleotides. Monitoring the interaction of the polymerase with the primed template nucleic acid molecule in the presence of a nucleotide molecule includes measuring dissociation kinetics of the polymerase from the primed template nucleic acid in the presence of any one of the four nucleotides.

The monitoring step can include monitoring the steady state interaction of the polymerase with the primed template nucleic acid molecule in the presence of the first nucleotide molecule, without chemical incorporation of the first nucleotide molecule into the primer of the primed template nucleic acid molecule. The monitoring can include monitoring dissociation of the polymerase with the primed template nucleic acid molecule in the presence of the first nucleotide molecule, without chemical incorporation of the first nucleotide molecule into the primer of the primed template nucleic acid molecule. The monitoring can include monitoring association of the polymerase with the primed template nucleic acid molecule in the presence of the first nucleotide molecule, without chemical incorporation of the first nucleotide molecule into the primer of the primed template nucleic acid molecule. Again, the test nucleotides in these procedures may be native nucleotides (i.e., unlabeled), labeled nucleotides (e.g., fluorescently labeled nucleotides), or nucleotide analogs (e.g., nucleotides modified to include reversible terminator moieties).

In SBB, either a chemical block on the 3′ nucleotide of the primer of the primed template nucleic acid molecule (e.g., a reversible terminator moiety on the base or sugar of the nucleotide), or the absence of a catalytic metal ion in the reaction mixture, or the absence of a catalytic metal ion in the active site of the polymerase prevents the chemical incorporation of the nucleotide into the primer of the primed template nucleic acid.

The identity of the next correct base or nucleotide can be determined by monitoring the presence, formation, and/or dissociation of the ternary complex. The identity of the next base may be determined without chemically incorporating the next correct nucleotide to the 3′-end of the primer. The identity of the next base can be determined by monitoring the affinity of the polymerase to the primed nucleic acid template in the presence of added nucleotides.

SBB can include an incorporation step. The incorporation step involves chemically incorporating one or more nucleotides at the 3′-end of a primer bound to a template nucleic acid. The incorporation reaction may be facilitated by an incorporation reaction mixture. The incorporation reaction mixture can have a different composition of nucleotides than the examination reaction. For example, the examination reaction can include one type of nucleotide and the incorporation reaction can include another type of nucleotide. By way of another example, the examination reaction comprises one type of nucleotide and the incorporation reaction comprises four types of nucleotides, or vice versa. The examination reaction mixture can be altered or replaced by the incorporation reaction mixture.

Nucleotides present in the reaction mixture but not sequestered in a ternary complex may cause multiple nucleotide insertions. A wash step can be employed prior to the chemical incorporation step to ensure only the nucleotide sequestered within a trapped ternary complex is available for incorporation during the incorporation step. The trapped polymerase complex may be a ternary complex, a stabilized ternary complex or ternary complex involving the polymerase, primed template nucleic acid and next correct nucleotide.

### Sequencing-by-Synthesis

Sequencing-by-synthesis generally involves the enzymatic extension of a nascent primer through the iterative addition of nucleotides against a template strand to which the primer is hybridized. Briefly, SBS can be initiated by contacting target nucleic acids, attached to sites in a flow cell, with one or more labeled nucleotides, DNA polymerase, etc. Those sites where a primer is extended using the target nucleic acid as template will incorporate a labeled nucleotide that can be detected. Detection can include scanning using an apparatus or method set forth herein. Optionally, the labeled nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the vessel (before or after detection occurs). Washes can be carried out between the various delivery steps. The cycle can be performed n times to extend the primer by n nucleotides, thereby detecting a sequence of length n. Exemplary SBS procedures, reagents and detection components that can be readily adapted for use with a method, system or apparatus of the present disclosure are described, for example, in Bentley et al., Nature 456:53-59 (2008), WO 04/018497; WO 91/06678; WO 07/123744; U.S. Pat. Nos. 7,057,026; 7,329,492; 7,211,414; 7,315,019 or 7,405,281, and US Pat. App. Pub. No. 2008/0108082 A1, each of which is incorporated herein by reference. Also useful are SBS methods that are commercially available from Illumina, Inc. (San Diego, Calif.). One or more reagents used in an SBS process can optionally be delivered via a mixed-phase fluid (e.g. a fluid foam, fluid slurry or fluid emulsion), contacted with a mixed-phase fluid, and/or removed by a mixed-phase fluid. A mixed-phase fluid can be removed from a flow cell for detection during an SBS process.

### Emulsion PCR

Emulsion PCR (EmPCR) is a commonly employed method for template amplification in multiple NGS-based sequencing platforms. The basic principle of emPCR is dilution and compartmentalization of template molecules in water droplets in a water-in-oil emulsion. Ideally, the dilution is to a degree where each droplet contains a single template molecule and functions as a micro-PCR reactor.

Emulsion PCR can comprise PCR amplification of an adaptor flanked shotgun DNA library in a water-in-oil emulsion. The PCR is multi-template PCR; in particular embodiments only a single primer pair is used. One of the PCR primers is tethered to the surface (5′ attached) of microscale beads. A low template concentration results in most bead-containing emulsion microvesicles having zero or one template molecule present. In productive emulsion microvesicles (an emulsion microvesicle where both a bead and template molecule are present), PCR amplicons can be captured to the surface of the bead. After breaking the emulsion, beads bearing amplification products can be selectively enriched. Each clonally amplified bead will bear on its surface PCR products corresponding to amplification of a single molecule from the template library. The beads can then be arrayed on a surface of a flow cell for sequencing. Various embodiments of emulsion PCR methods are set forth in Dressman et al., Proc. Natl. Acad. Sci. USA 100:8817-8822 (2003), PCT Patent Publ. No. WO 05/010145, U.S. Patent Publ. Nos. 2005/0130173, 2005/0064460, and 2005/0042648, the content of each of which is incorporated herein by reference in its entirety.

Beads can be configured for parallel sequencing of multiple nucleic acids. In multiplex formats wherein multiple nucleic acid species are sequenced in parallel, clonally amplified target sequences (e.g., via emulsion PCR (emPCR) or bridge amplification) are typically covalently immobilized on a substrate. For example, when practicing emulsion PCR, the target of interest is immobilized on a bead substrate, whereas clonally amplified targets are immobilized in channels of a flowcell based substrate or specific locations on an array based or chip based substrate.

### EXAMPLES

Some aspects of the embodiments discussed above are disclosed in further detail in the following examples, which are not in any way intended to limit the scope of the present disclosure.

### Example 1

### Colloidal Self-Assembly

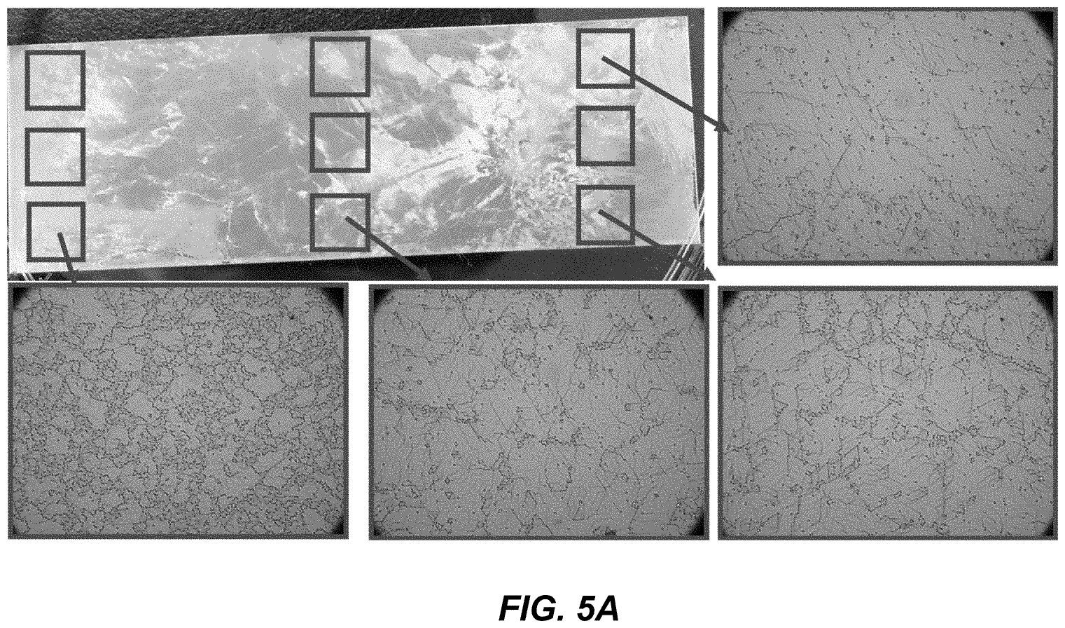

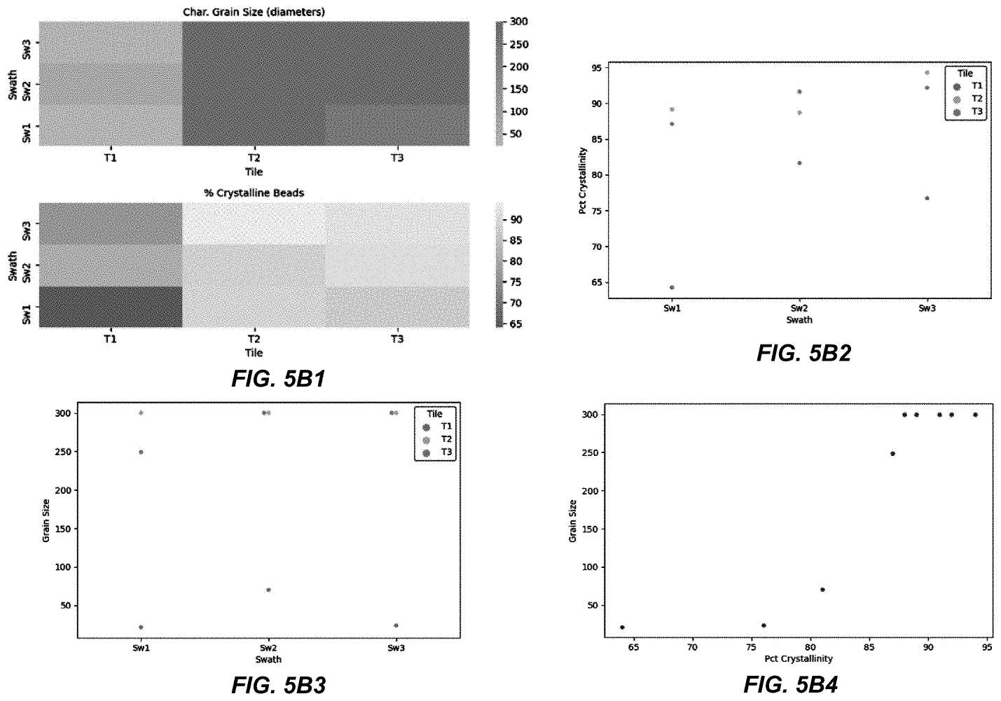

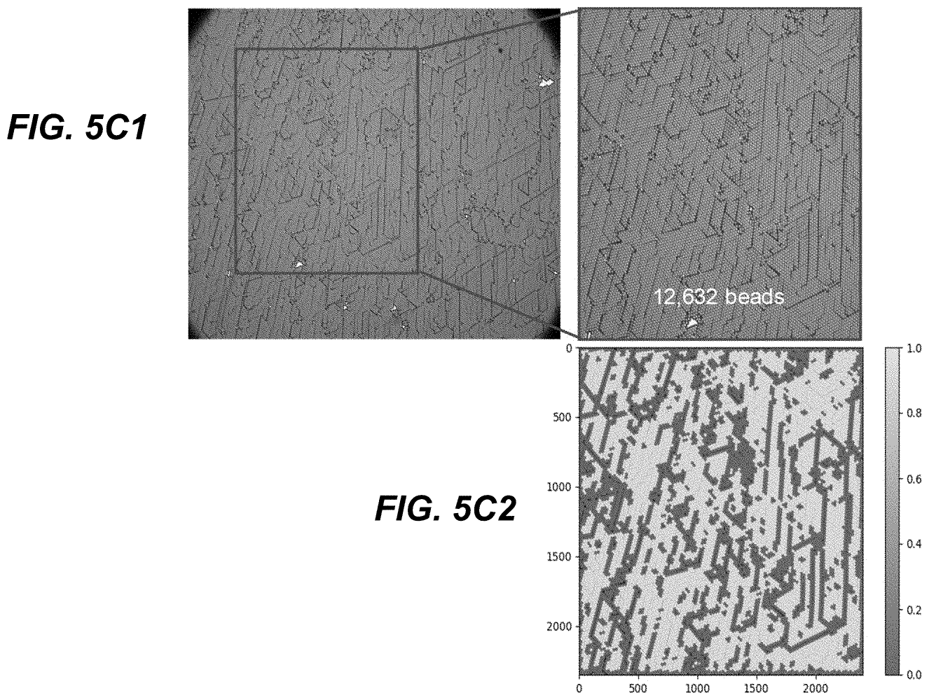

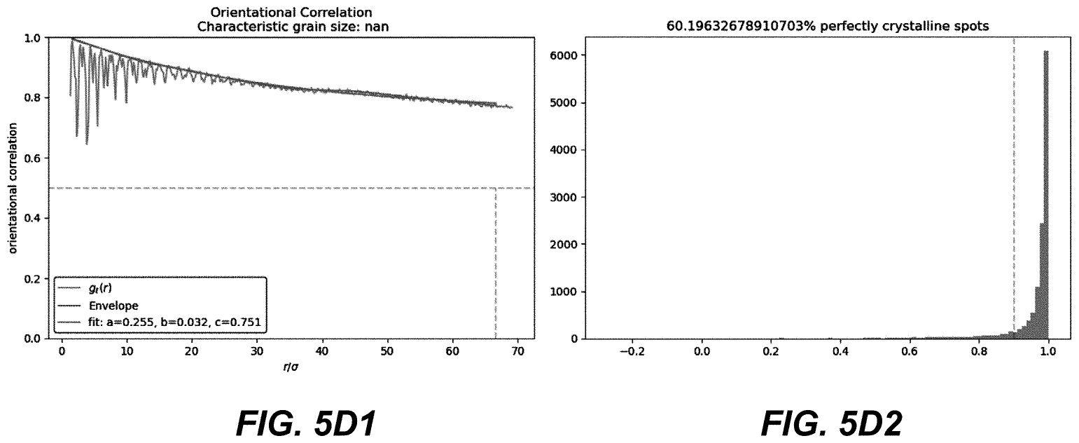

FIG. 5A shows an image of a surface with beads loaded thereon as described herein. Beads were introduced into a liquid and were allowed to pack with colloidal self-assembly. The packed beads were loaded onto a surface. The beads at different areas of the surface had different characteristics (see the enlarged images of the surface in FIG. 5A). The heatmap and plots in FIGS. 5B1-5B3 show that the grain size of the packed beads and crystallinity of the packed beads between different areas of the surface. The plot in FIG. 5B4 shows that there was a correlation between bead crystallinity (ordered, regularly positioned, well packed beads) and the grain size of the packed beads such that large grain sizes were achieved with high bead crystallinities. FIG. 5C1 shows an image of beads loaded onto a surface as described herein with an area enlarged showing variations of bead crystallinity. FIG. 5C2 is a heat map showing variation of bead crystallinity of the enlarged area shown in FIG. 5C1. FIG. 5D1 is a plot showing the characteristics of the beads loaded onto the surface. FIG. 5D2 is a bar chart showing a distribution of the crystallinity of the beads loaded onto the surface. Grain size in the context of FIG. 5 is a length scale that reflects the characteristic (average) grain size of monocrystalline domains in a microscope image such as is shown in FIG. 5A. The unit is in bead diameters (r/a, where a is the bead diameter) and since the size of the beads in FIG. 5A is 1 μm, the grain size in this case is in units of μm. This metric is derived from plots like the one shown in FIG. 5D1, where the grain size is defined by the length scale (r/a) at which the orientational correlation (metric that captures quality of long-range order) decreases to 0.5. It should be noted that certain microscope images only contain one single crystalline domain, and thus the orientational correlation never decays below 0.5, leading to an infinite grain size, which we then capped at 300 μm since it is the length of the diagonal of the microscope image (the largest length we can measure in the field of view of the microscope image).

### Example 2

### Etching

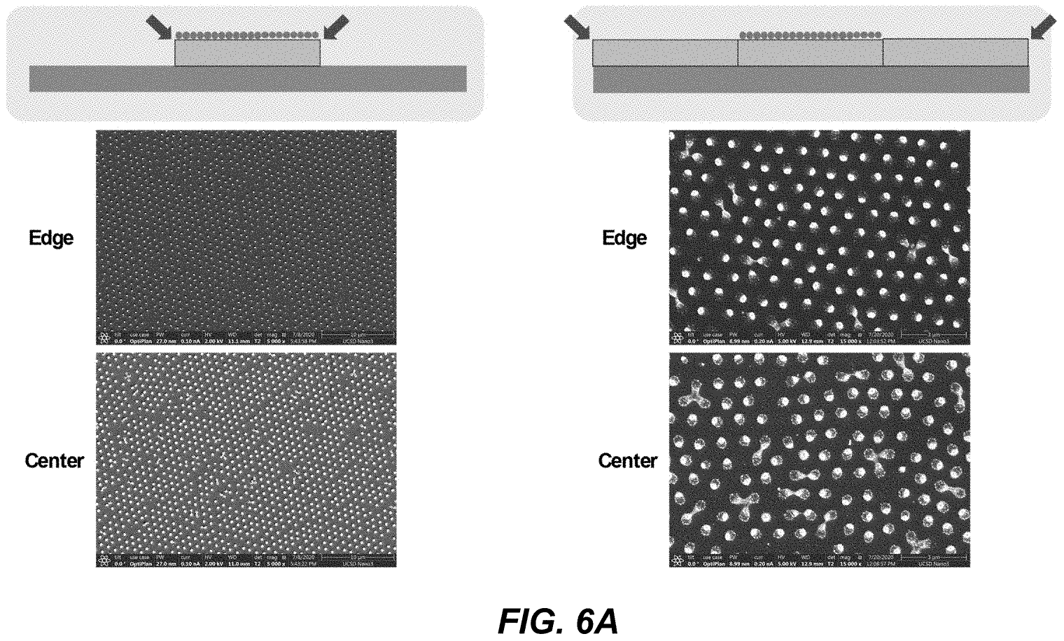

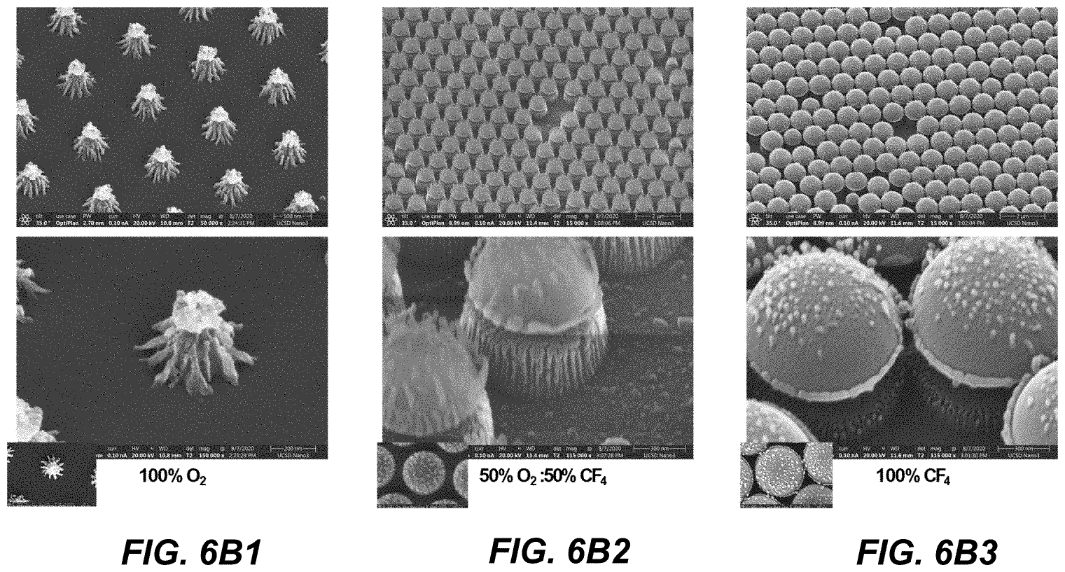

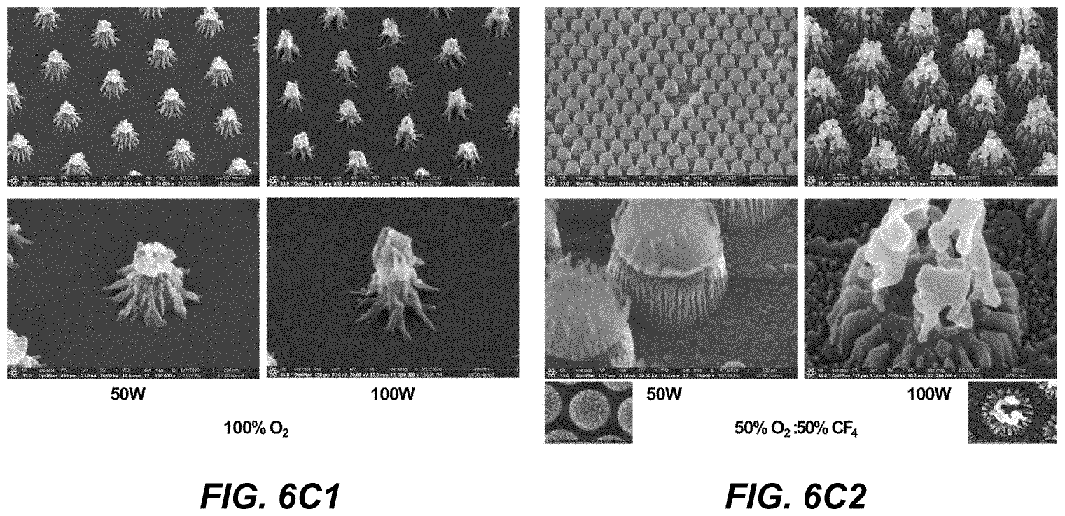

FIGS. 6A and 6B1-6B3 show that with oxygen plasma etching, the etch rate varied across the planar surface.

The SEM images in FIGS. 6B1-6B3 show the effect of the etching gas on reactive ion etching (50 W, 10 mT, 8 min, 10° C.) with 100% oxygen (FIG. 6B1), 50% oxygen and 50% carbon tetrafluoride (FIG. 6B2), and 100% carbon tetrafluoride (FIG. 6B3). The circular region shown at the top of individual structures is residual polystyrene from the bead, while the plateau underneath is the AZ photoresist layer (AZ1500 series is a commercial photoresist by MicroChemicals GmbH). The functional layer is covered by the AZ photoresist layer, and the surrounding area is exposed glass (potentially with residue from the resist layer). While the image of this figure shows etching of an AZ1500 photoresit layer, it will be understood that other resist (e.g., photoresists) can be etched according to the methods described herein. The SEM images in FIGS. 6C1 and 6C2 show the effect of radio frequency (RF) power on reactive ion etching (10 mT, 8 min, 10° C.) using 100% oxygen (FIG. 6C1), and 50% oxygen and 50% carbon tetrafluoride (FIG. 6C2). The SEM images in FIG. 6D shows the result of the planar surface using a two-step etching process:

(1) more isotropic (tune PS Φ): 50 W, 20 mT, 10 sccmO2, 4 min, 10° C.

(2) more anisotropic (etch through AZ and reduce Φ): 50 W, 10 mT, 10 sccmO₂:10 sccmCF₄, 4 min, 10° C.

This first etching step was not performed longer because O2 etches AZ faster than PS (under-cut etch). The second etching step did not just use CF₄ as the etching agent in order to have lateral and vertical etch at the same time.

### Example 3

### emPCR Bead Deposition and Sequencing

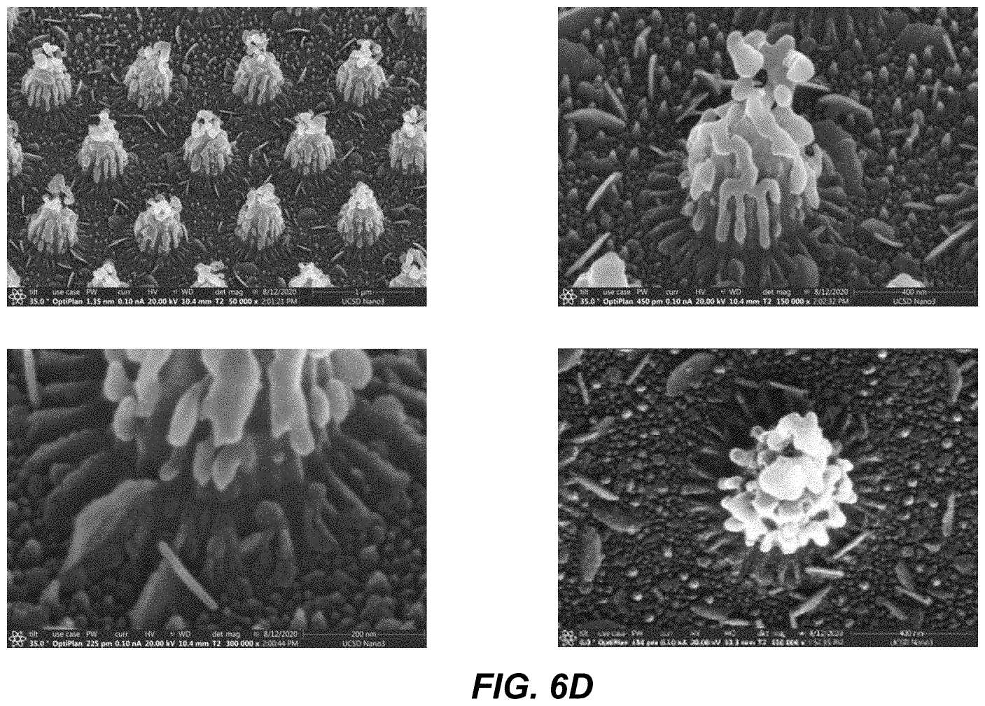

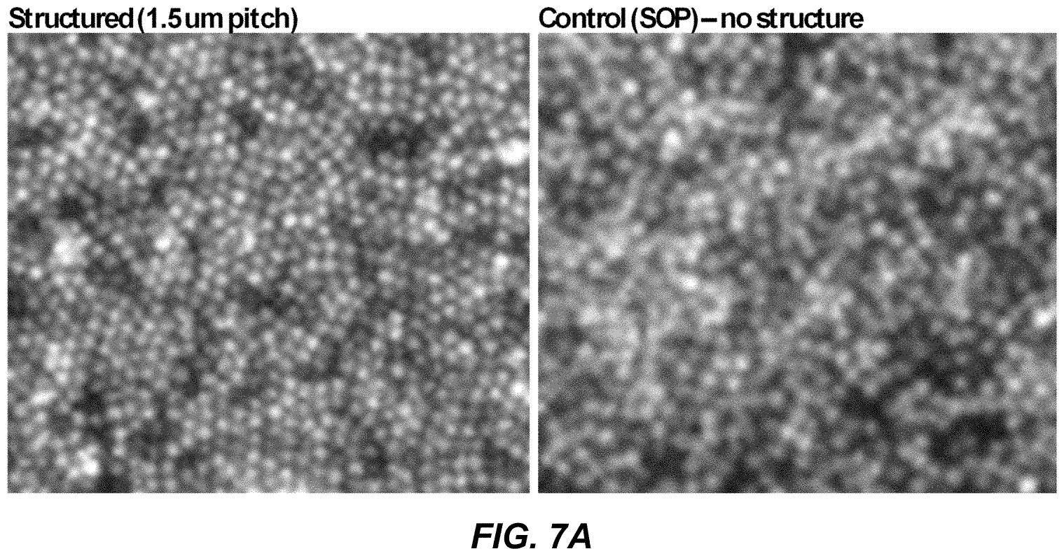

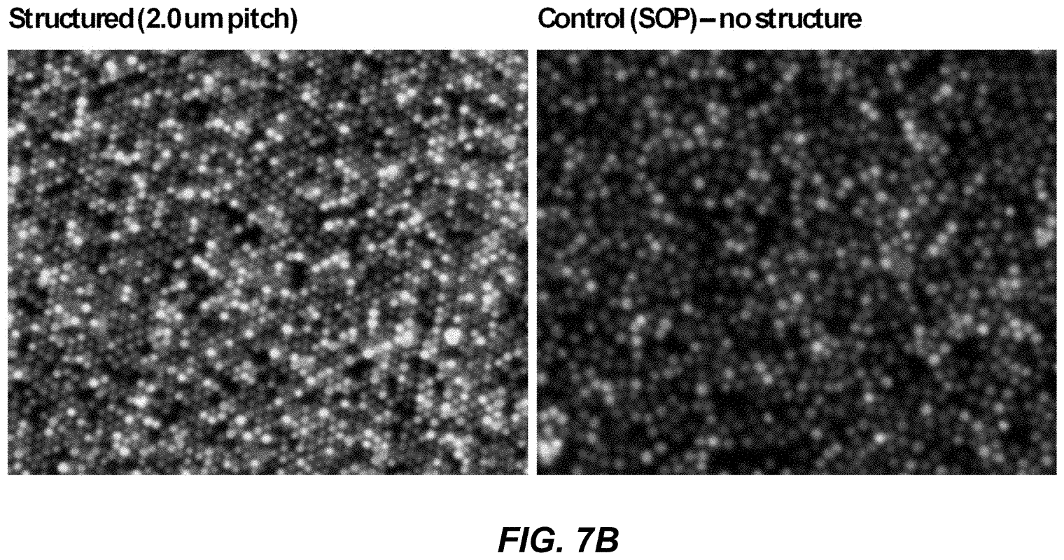

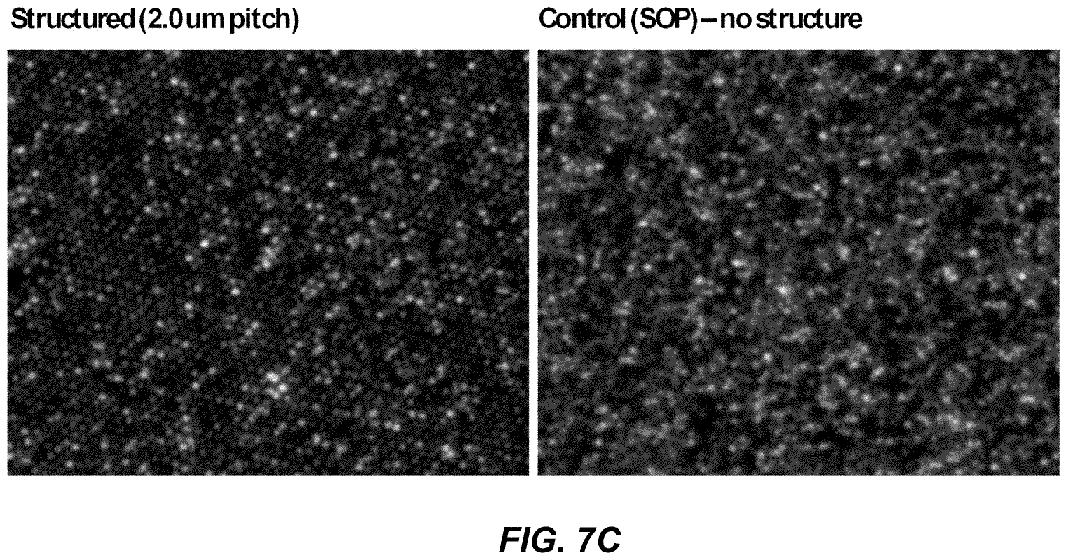

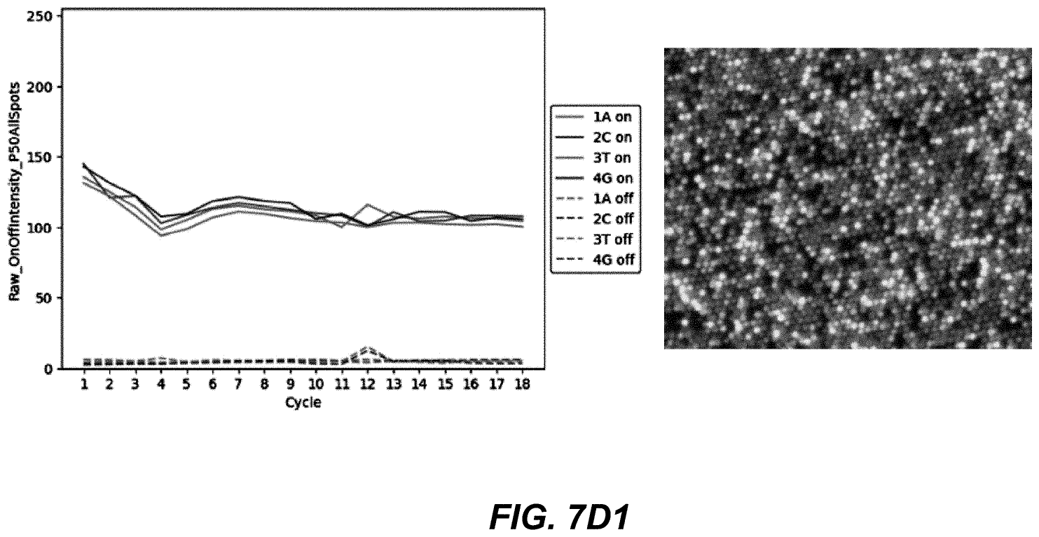

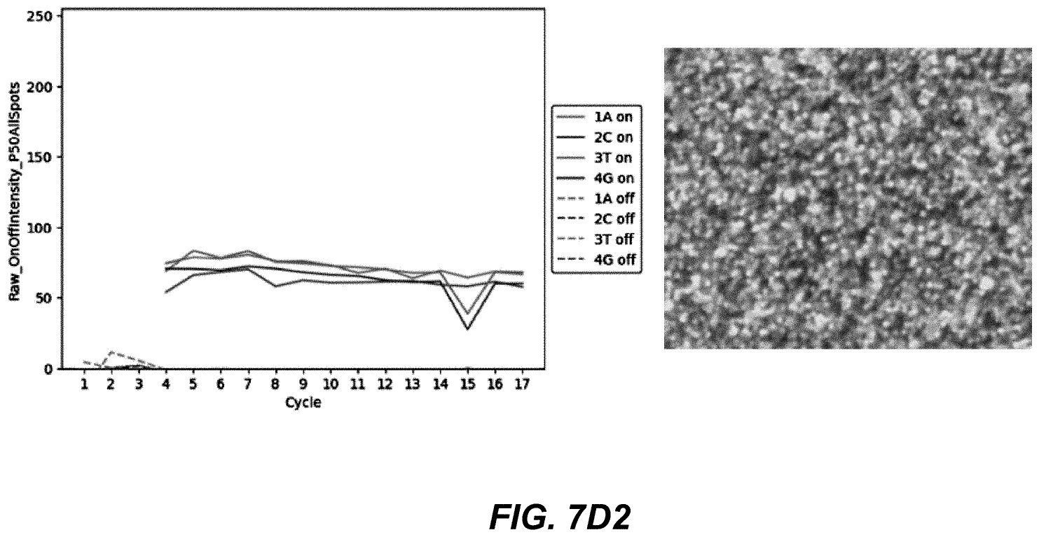

The fluorescent microscopy images in FIG. 7A compare sequencing with emPCR beads absorbed onto a 1.5-μm pitch structured aminosilane surface (left image) and a non-structured surface (right image). The fluorescent images in FIGS. 7B-7C compare sequencing with emPCR beads absorbed onto a 2-μm pitch structured aminosilane surface (left images) and a non-structured surface (right images). The plots and fluorescent images in FIGS. 7D1 and 7D2 compare sequencing with emPCR beads absorbed onto a 2-μm pitch structured aminosilane surface (FIG. 7D1) and a non-structured surface (FIG. 7D2). FIGS. 7A-7C and 7D1-7D2 show that with structured aminosilance surfaces, the fluorescent signals from the emPCR beads were more ordered, tightly packed, and better separated and the sequencing results were better.

### Execution Environment

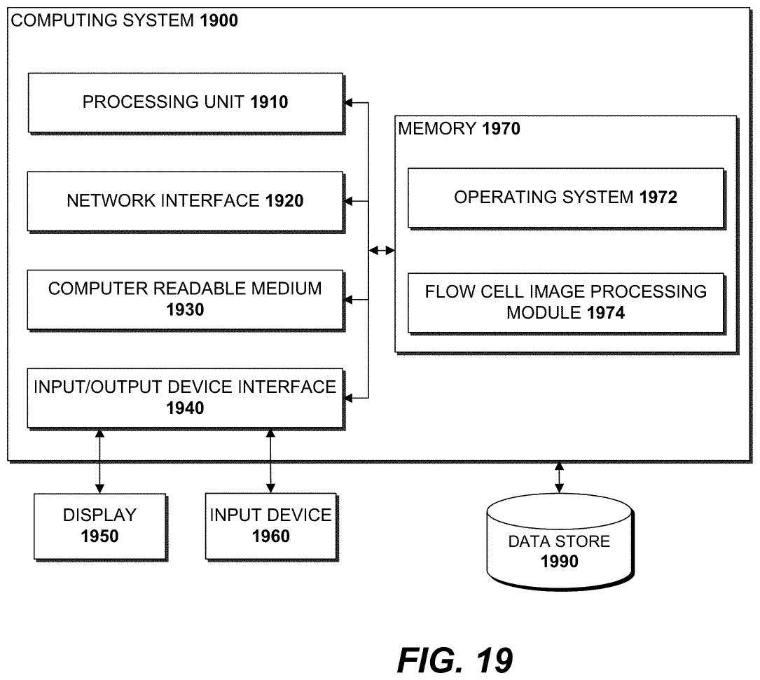

FIG. 19 depicts a general architecture of an example computing device 1900 configured to execute the processes and implement the features described herein. The general architecture of the computing device 1900 depicted in FIG. 19 includes an arrangement of computer hardware and software components. The computing device 1900 may include many more (or fewer) elements than those shown in FIG. 19. It is not necessary, however, that all of these generally conventional elements be shown in order to provide an enabling disclosure. As illustrated, the computing device 1900 includes a processing unit 1910, a network interface 1920, a computer readable medium drive 1930, an input/output device interface 1940, a display 1950, and an input device 1960, all of which may communicate with one another by way of a communication bus. The network interface 1920 may provide connectivity to one or more networks or computing systems. The processing unit 1910 may thus receive information and instructions from other computing systems or services via a network. The processing unit 1910 may also communicate to and from memory 1970 and further provide output information for an optional display 1950 via the input/output device interface 1940. The input/output device interface 1940 may also accept input from the optional input device 1960, such as a keyboard, mouse, digital pen, microphone, touch screen, gesture recognition system, voice recognition system, gamepad, accelerometer, gyroscope, or other input device.

The memory 1970 may contain computer program instructions (grouped as modules or components in some embodiments) that the processing unit 1910 executes in order to implement one or more embodiments. The memory 1970 generally includes RAM, ROM and/or other persistent, auxiliary or non-transitory computer-readable media. The memory 1970 may store an operating system 1972 that provides computer program instructions for use by the processing unit 1910 in the general administration and operation of the computing device 1900. The memory 1970 may further include computer program instructions and other information for implementing aspects of the present disclosure.

For example, in one embodiment, the memory 1970 includes a flow cell image processing module 1974 for processing one or more flow cell images, such as aligning flow cell images, orienting flow cell images, sorting flow cell images, and performing quality assessment on flow cell images. In addition, memory 1970 may include or communicate with the data store 1990 and/or one or more other data stores that store the flow cell images, processed flow cell images, or sequencing results.

### ADDITIONAL CONSIDERATIONS

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Accordingly, phrases such as “a device configured to” are intended to include one or more recited devices. Such one or more recited devices can also be collectively configured to carry out the stated recitations. For example, “a processor configured to carry out recitations A, B and C can include a first processor configured to carry out recitation A and working in conjunction with a second processor configured to carry out recitations B and C. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

All of the processes described herein may be embodied in, and fully automated via, software code modules executed by a computing system that includes one or more computers or processors. The code modules may be stored in any type of non-transitory computer-readable medium or other computer storage device. Some or all the methods may be embodied in specialized computer hardware.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example through multi-threaded processing, interrupt processing, or multiple processors or processor cores or on other parallel architectures, rather than sequentially. In addition, different tasks or processes can be performed by different machines and/or computing systems that can function together.

As used herein, the term “about” a number refers to a range spanning +/−10% of that number, and “about” a range refers to an extended range spanning from +/−10% of the listed endpoints.

As used herein, the term “planar” refers to a surface that is locally smooth or alternately that has a locally defined tangent. Many of the embodiments herein are contemplated for use involving a flat surface, for example so as to facilitate imaging of signaling from the surface without change in focus even if the surface is translated or slid relative to the imaging device. However, the term should not in all cases be limited to these embodiments, as the technology is in some cases also consistent with application to surfaces that are locally planar but globally spherical, such as beads, or that are locally planar but attached to surfaces that are not uniformly smooth, such as surfaces having wells, peaks, corners, or other discontinuities on surface smoothness.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

Any process descriptions, elements or blocks in the flow diagrams described herein and/or depicted in the figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure and protected by the following claims.

1.-322. (canceled)

323. A flow cell surface comprising a plurality of binding sites of at least 10,000 binding sites separated by disjunctions,
wherein the disjunctions are irregular,
wherein the position, size, and shape of the disjunctions are not predetermined, and
wherein binding sites of the flow cell are hydrophilic, hydrophobic, positively charged, negatively charged, comprise a functional moiety for covalent attachment, comprise an affinity biomolecule, comprise an intermediate nucleic acid, or a combination thereof.

324. The flow cell surface of claim 323, wherein the pitch between any two adjacent binding sites is at most 2 micrometers.

325. The flow cell surface of claim 324, wherein the pitch between a middle binding site and a neighbor binding site of the plurality of binding sites is at least 0.1 m different than the pitch between the middle binding site and another neighbor binding site of the plurality of binding sites.

326. The flow cell surface of claim 323, wherein a material of the flow cell surface comprises silicon, silicon nitride glass, borosilicate glass, quartz, fused quartz, silica, fused silica, a metal, a ceramic, plastic, or a combination thereof.

327. The flow cell surface of claim 323, wherein the plurality of binding sites comprises a plurality of nucleic acids.

328. The flow cell surface of claim 327, wherein one, at least 50% of the binding sits comprise a single monoclonal nucleic acid cluster.

329. The flow cell surface of claim 327, wherein one, one or more, or each of the plurality of nucleic acids comprises an amplification primer or a splint primer, an amplification product from rolling circle amplification (RCA), a DNA tile, or a combination thereof.

330. A method of specifying binding sites on a planar structure comprising:
providing a planar structure subsumed in a liquid;
delivering a plurality of particles to a surface of the liquid; and
removing the liquid between the plurality of particles and the planar structure, such that the plurality of particles is in contact with the planar structure,
wherein the plurality of particles in contact with the planar structure specifies a plurality of binding sites on the planar structure,
wherein the plurality of binding sites comprises at least 10,000 binding sites which are separated by disjunctions,
wherein the disjunctions are irregular, and
wherein the position, size, and shape of the disjunctions are not predetermined.

331. The method of claim 330, wherein the pitch between any two adjacent particles is at most 2 micrometers.

332. The method of claim 331, wherein the plurality of particles comprises a first subset of particles having a first identical radius and a second subset of particles having a second identical radius, and wherein the first identical radius and the second identical radius are different by at least 0.1 um.

333. The method of claim 330, further comprising etching the planar structure and the plurality of particles in contact with the planar structure to generate:
a plurality of retained regions where a substance layer on the planar surface is in contact with the plurality of particles and is differentially retained, and
a plurality of etched regions where the substance is not in contact with the plurality of particles and is differentially removed.

334. The method of claim 333, wherein the planar surface comprises a masking layer in contact with the substance layer, wherein the substance layer on the planar surface at the plurality of retained regions is in contact with the plurality of particles via the masking layer at the plurality of retained regions, wherein the masking layer at the plurality of retained regions is in contact with the plurality of particles and is differentially retained, and wherein the masking layer at the plurality of etched regions is not in contact with the plurality of particles and is differentially removed.

335. The method of claim 334, further comprising applying a passivating chemistry to interstitials of binding sites corresponding to retained regions of the plurality of retained regions being generated while the beads prevent grafting of functional groups to the binding sites.

336. The method of claim 335, further comprising removing the masking layer and exposing bare glass of the planar structure, wherein a secondary chemical vapor deposition (CVD) process is used to graft active chemistry for DNA covalent capture on the binding sites.

337. The method of any one of claim 333, comprising passivating the plurality of etched regions to generate passivated regions.

338. A method comprising delivering a plurality of nucleic acids to a plurality of binding sites on a surface of a flow cell, wherein the plurality of binding sites comprising at least 10,000 binding sites separated by disjunctions, wherein the disjunctions are irregular, and wherein the position, size, and shape of the disjunctions are not predetermined.

339. The method of claim 338, wherein the nucleic acids are concatemers that are rolling circle amplification (RCA) products.

340. The method of claim 339, further comprising performing rolling circle amplification (RCA) in solution by extending an amplification primer or a splint primer with a plurality of templates to generate the concatemers, prior to depositing the concatemers on the binding sites.

341. The method of claim 339, wherein the concatemer is bound to an intermediate nucleic acid, and wherein the intermediate nucleic acid is a DNA tile.

342. The method of claim 339, comprising forming concatemers on the binding sites of the flow cell by rolling circle amplification (RCA) of a target nucleic acid from splint oligonucleotides.

343. The method of claim 342, wherein the splint oligonucleotides are each presented by a DNA tile bound to a binding site of the plurality of binding sites.

344. The method of claim 343, wherein the DNA tile comprises a plurality of capture sequences each comprising a portion of a splint oligonucleotide.

345. The method of claim 338, comprising:
providing a plurality of asymmetric DNA tiles;
providing a plurality of concatemers;
binding individual concatemers to a first side of individual DNA tiles in solution; and
depositing the DNA tiles on the surface of the flow cell such that a second side of the individual DNA tiles binds to the binding sites of the surface while the first side of the individual DNA tiles remains bound to a concatemer.

346. The method of claim 338, wherein at least 50% of the binding sits comprise a single monoclonal nucleic acid cluster.

347. The method of claim 339, further comprising sequencing the concatemers.
